(19) United States
(12) Patent Application Publication
(10) Pub. No.: US 2026/0048076 A1
(43) Pub. Date: Feb. 19, 2026

(54) METHODS FOR DECREASING OR INHIBITING HUMAN L1 RETROTRANSPOSON RNA AND COMPOSITIONS FOR USE THEREIN

(71) Applicant: ALTOS LABS, INC., Redwood City, CA (US)

(72) Inventors: Pradeep Reddy DUBBAKA VENU, San Diego, CA (US); Juan Carlos IZPISUA BELMONTE, La Jolla, CA (US)

(73) Assignee: ALTOS LABS, INC., Redwood City, CA (US)

(21) Appl. No.: 19/102,318

(22) PCT Filed: Aug. 8, 2023
(86) PCT No.: PCT/US2023/071853

§ 371 Date: Feb. 7, 2025

Related U.S. Application Data
(60) Provisional application No. 63/370,914, filed on Aug. 9, 2022.

(51) Int. Cl. A61K 31/7115; A61K 9/00; A61K 31/712; A61K 31/7125; A61K 45/06; A61P 3/04; C12N 15/113; C12N 15/86

(52) U.S. Cl. CPC A61K 31/7115; A61K 9/0019; A61K 31/712; A61K 31/7125; A61K 45/06; A61P 3/04; C12N 15/113; C12N 15/86; C12N 2310/11; C12N 2310/315; C12N 2310/3341; C12N 2310/335

(57) ABSTRACT

Methods for decreasing or inhibiting human L1 retrotransposon RNA and compositions for use therein.

### CROSS-REFERENCE TO RELATED APPLICATIONS

This application claims the benefit of U.S. Provisional Application No. 63/370,914, filed Aug. 9, 2022, which is incorporated by reference herein in its entirety.

### FIELD OF THE INVENTION

The disclosure relates to compositions and methods for reducing LINE-1 retrotransposon RNA in a subject.

### BACKGROUND OF THE INVENTION

Loss of heterochromatin occurs during physiological and pathological aging, and premature aging genetic disorders such as Hutchinson-Gilford progeria syndrome (HGPS) and Werner syndrome (WRN) are characterized by disorganized heterochromatin. Long interspersed nuclear elements (LINEs) are a group of non-LTR (long terminal repeat) retrotransposons that are widespread in the genome of many eukaryotes. LINEs make up a family of transposons, where each LINE is about 7000 base pairs long. LINEs are transcribed into mRNA and translated into protein that acts as a reverse transcriptase. The reverse transcriptase makes a DNA copy of the LINE RNA that can be integrated. The only abundant LINE in humans is LINE-1 (“L1”), which accounts for about 21% of the human genome. Endogenous L1 elements are transcriptionally active in both pathological (progeroid syndromes) and physiologically aged cells. LINE-1 has two open reading frames (ORF1 and ORF2), and expresses multiple proteins, such as a reverse transcriptase.

### SUMMARY OF THE INVENTION

The invention relates to oligonucleotides, pharmaceutical compositions, and methods of decreasing or inhibiting human L1 retrotransposon RNAs. Such oligonucleotides and pharmaceutical compositions comprising the oligonucleotides of the invention can be used in the treatment of progeria syndromes or signs and/or symptoms of aging. In various aspects, the invention includes administering a composition comprising an antisense oligonucleotide described herein to a subject having a progeria syndrome or signs and/or symptoms of aging in an amount effective to treat, prevent, or inhibit premature aging or an age-related disease in the subject. The invention also relates to rejuvenating a tissue in a subject experiencing premature aging or having an age-related disease, or reversing signs or symptoms of aging in a tissue of a subject. The antisense oligonucleotides of the invention can inhibit or decrease LINE-1 RNA in a cell.

One aspect disclosed herein relates to an oligonucleotide consisting of the sequence of SEQ ID NO: 1, wherein at least one nucleoside is 2′ fluoroarabinonucleic acid (FANA) modified.

In some aspects, at least two nucleotides of the oligonucleotide are 2′ FANA modified.

In some aspects, 3 to 21 nucleosides of the oligonucleotide are 2′ FANA modified.

In some aspects, at least ten nucleosides of the oligonucleotide are 2′ FANA modified.

In some aspects, 21 nucleotides of the oligonucleotide are 2′ FANA modified.

In some aspects, at least two 2′ FANA modified nucleosides are located at the 5′ end or the 3′ end or both of the oligonucleotide.

In some aspects, five 2′ FANA modified nucleosides are located at the 5′ end of the oligonucleotide and five 2′ FANA modified nucleosides are located at the 3′ end of the oligonucleotide and the remaining nucleotides are not 2′ FANA modified.

In some aspects, the oligonucleotide comprises at least one alternative internucleoside linkage.

In some aspects, the at least one alternative internucleoside linkage is selected from a phosphorothioate internucleoside linkage and an alkyl phosphate internucleoside linkage.

In some aspects, the oligonucleotide comprises at least one alternative nucleobase.

In some aspects, the at least one alternative nucleobase is selected from a 5-substituted pyrimidine, 6-azapyrimidine, pseudouridine, N-2 substituted purine, N-6 substituted purine, or O-6 substituted purine.

In some aspects, the at least one alternative nucleobase is 5′-methylcytosine or 5′methoxyuridine.

Further provided is a pharmaceutical composition comprising one or more oligonucleotides disclosed herein and a pharmaceutically acceptable carrier or excipient.

Also provided is a composition comprising one or more of the oligonucleotides disclosed herein and a lipid nanoparticle, a polyplex nanoparticle, a lipoplex nanoparticle, or a liposome.

Also provided is a polynucleotide comprising a nucleotide sequence comprising one or more of the oligonucleotides disclosed herein and further comprising a regulatory nucleotide sequence that controls expression of the one or more oligonucleotide.

Further provided is a vector comprising such polynucleotide. In some aspects, the vector is selected from a DNA plasmid, a viral vector, a bacterial vector, a cosmid, or an artificial chromosome.

Further provided is a method of treating, preventing, or inhibiting premature aging or an age-related disease in a subject, the method comprising administering a therapeutically effective amount of an oligonucleotide disclosed herein, pharmaceutical composition disclosed herein, or a composition disclosed herein to the subject in an amount effective to treat, prevent, or inhibit premature aging or an age-related disease in the subject.

In some aspects, the subject has a progeroid syndrome. In some aspects, the progeroid syndrome is selected from the group consisting of Hutchinson-Gilford progeria syndrome; Werner syndrome; atypical progeria; mandibuloacral dysplasia type A; mandibuloacral dysplasia type B; mandibuloacral dysplasia associated to MTX2; MDPL (mandibular hypoplasia, deafness, progeroid features and lipodystrophy syndrome); Nestor-Guillermo progeria syndrome; and restrictive dermopathy.

In some aspects, the subject has a mutation in a LMNA gene, ZMPSTE24 gene, BANF1 gene, POLD1 gene, MTX2 gene, or WRN gene.

In some aspects, the method further comprises administering to the subject an additional therapeutic agent. In some aspects, the additional therapeutic agent is an aging-related therapeutic agent.

In some aspects, the therapeutically effective amount is administered by an intravenous, intramuscular, intradermal, subcutaneous, intraperitoneal, or intranasal route.

Further provided is a method of treating, preventing, or inhibiting signs or symptoms of aging in a subject, the method comprising administering a therapeutically effective amount composition disclosed herein to the subject in an amount effective to treat, prevent, or inhibit signs or symptoms of aging in the subject.

In some aspects, the method further comprises administering an additional therapeutic agent.

In some aspects, the additional therapeutic agent is an aging-related therapeutic agent. In some aspects, the therapeutically effective amount is administered by an intravenous, intramuscular, intradermal, subcutaneous, intraperitoneal, or intranasal route.

Also provided is a method of decreasing LINE-1 RNA in a subject, the method comprising administering a therapeutically effective amount of an oligonucleotide as disclosed herein, a pharmaceutical composition as disclosed herein, or a composition as disclosed herein to the subject.

In some aspects, the method of decreasing LINE-1 RNA in a subject further comprises administering an additional therapeutic agent. In some aspects, the additional therapeutic agent is an aging-related therapeutic agent. In some aspects, the therapeutically effective amount is administered by an intravenous, intramuscular, intradermal, subcutaneous, intraperitoneal, or intranasal route.

Also provided is a method of rejuvenating a tissue in a subject experiencing pre-mature aging or having an age-related disease, the method comprising administering a therapeutically effective amount of an oligonucleic as disclosed herein, a pharmaceutical composition as disclosed herein, or a composition as disclosed herein to the subject.

In some aspects, the method of rejuvenating a tissue further comprises administering an additional therapeutic agent.

In some aspects, the additional therapeutic agent is an aging-related therapeutic agent. In some aspects, the therapeutically effective amount is administered by an intravenous, intramuscular, intradermal, subcutaneous, intraperitoneal, or intranasal route.

Further provided is a method of reversing signs or symptoms of aging in a tissue of a subject, the method comprising administering a therapeutically effective amount of an oligonucleotide, a pharmaceutical composition, or a composition as disclosed herein to the subject.

In some aspects, the method of rejuvenating a tissue further comprises administering an additional therapeutic agent.

In some aspects, the additional therapeutic agent is an aging-related therapeutic agent. In some aspects, the therapeutically effective amount is administered by an intravenous, intramuscular, intradermal, subcutaneous, intraperitoneal, or intranasal route.

In some aspects, the subject is a human, mouse, rat, horse, dog, or cat.

In some aspects, an oligonucleotide as disclosed herein exhibits at least 50% reduction in expression of senescence-associated secretory phenotype (SASP) genes in a progeroid human cell at a 1-5 μM single-stranded oligonucleotide concentration when determined using a human progeroid cell assay when compared with a control human progeroid cell.

In some aspects, the progeroid human cell is a LMNA−/− cell or a WRN−/− cell.

Also provided is a kit comprising: (i) an oligonucleotide as disclosed herein, a pharmaceutical composition as disclosed herein, or a composition as disclosed herein and (ii) instructions for administering the oligonucleotide, the pharmaceutical composition or the composition to a subject in need thereof.

In some aspects, the kit further comprises a device for administering the oligonucleotide, the pharmaceutical composition, or the composition disclosed herein.

### BRIEF DESCRIPTION OF THE DRAWINGS/FIGURES

FIG. 1A shows a TaqMan qPCR analysis of LINE-1-Ta elements in WT, HGPS, and WRN hMSCs, P values were calculated using multiple t-test with Holm-Sidak correction. FIG. 1B shows a TaqMan qPCR analysis of LINE-1-Ta elements in progeroid syndromes driven by LMNA E578V, R644C, and T10I mutations, P values were calculated using multiple t-test with Holm-Sidak correction. FIG. 1C shows a TaqMan qPCR analysis of LINE-1-Ta elements in a patient with atypical progeroid syndrome (APS) driven by unknown mutation, P values were calculated using multiple t-test with Holm-Sidak correction. FIG. 1D shows in the left panel: an immuno-FISH analysis of LINE-1 RNA, H3K9me3, and H3K27me3 in WT and HGPS human dermal fibroblasts (HDFs) at passage 11 (early passage) and passage 20 (late passage); in the center panel: a fluorescence signal quantification was performed on 100 nuclei for each biological replicate, nuclei were counterstained with DAPI (blue), scale bar, 5 μm, P values were calculated using unpaired t-test with Welch's correction, n=3; and in the right panel: an ELISA assay of H3K9me3 and H3K27me3 quantification on 10 μg of protein extracts, n=4, P values were calculated using multiple t-test with Holm-Sidak correction. FIG. 1E shows a qPCR analysis of SASP genes (p16, p21, ATF3, MMP13, BTG2 and GADD45b) in WT and HGPS HDFs at early and late passage, P values were calculated using multiple t-test with Holm-Sidak correction. FIG. 1F shows a senescence associated β-galactosidase activity assay in HGPS HDFs at early and late passage, P values were calculated using unpaired t-test with Welch's correction. FIG. 1G shows a TaqMan qPCR analysis of LINE-1 and alpha-satellite DNA expression in WT and HGPS HDFs at early and late passage, P values were calculated using unpaired t-test with Welch's correction. FIG. 1H shows a qPCR analysis of SUV39H1 and SUV39H2 RNA immunoprecipitation (RIP) for 5′UTR-ORF1 and ORF2-3′UTR regions of LINE-1 RNA in passage 12 HDFs, P values were calculated using unpaired t-test with Welch's correction. FIG. 1I shows an ELISA assay of SUV39H1 histone methyl transferase (HMT) activity in the presence (violet) or absence (grey) of LINE-1 RNA. Antisense LINE-1 RNA (pink) was used as a negative control, P values were calculated using multiple t-test with Holm-Sidak correction. Data are represented as mean with SEM with individual values plotted within bars.

FIG. 2A shows a qPCR analysis of SASP genes (p16, p21, ATF3, MMP13, BTG2 and GADD45b) in WT and HGPS hMSCs untreated (N.T.) and scramble (Scr) or LINE-1 (L1) antisense oligonucleotides (ASO) treated, P values were calculated using multiple t-test with Holm-Sidak correction. FIG. 2B shows a qPCR analysis of SASP genes (p16, p21, ATF3, MMP13, BTG2 and GADD45b) in WT and WRN hMSCs untreated (N.T.) and scramble (Scr) or LINE-1 (L1) antisense oligonucleotides (ASO) treated, P values were calculated using multiple t-test with Holm-Sidak correction. FIG. 2C shows in the left panel: an immuno-FISH analysis of LINE-1 RNA, H3K9me3, and H3K27me3 in HGPS and WRN hMSCs treated with Scr or L1 ASO; and in the right panel: a fluorescence signal quantification were performed on 100 nuclei for each biological replicate, nuclei were counterstained with DAPI, scale bar, 50 μm, P values were calculated using unpaired t-test with Welch's correction, n=3. FIG. 2D shows a senescence-associated β-galactosidase activity assay in HGPS and WRN hMSCs treated with Scr ASO and L1 ASO, P values were calculated using multiple t-test with Holm-Sidak correction. FIG. 2E shows a Western blot analysis of chromatin fractionation in WT, HGPS untreated, Scr ASO and L1, ASO treated cells, as a fractionation quality control LAMIN B antibody was used to mark the nuclear matrix-associated proteins fraction, HDAC2 for the chromatin-bound and nucleosol protein fractions and GAPDH for the cytosol protein fraction. FIG. 2F shows an ELISA quantification of protein extract derived from chromatin fractionation experiment shown in panel (E). 1 g of protein from each fraction was used as input, P values were calculated using multiple t-test with Holm-Sidak correction. FIG. 2G shows an immuno-FISH analysis of LINE-1 RNA, H3K9me3, and H3K27me3 in WT and HGPS HDFs treated with Scr or L1 ASO, fluorescence signal quantification were performed on 100 nuclei for each replicate, nuclei were counterstained with DAPI, scale bar, 5 μm.

FIG. 3A shows a measurement of DNA methylation age of HGPS and WRN cells using epigenetic clocks “Skin and Blood Clock (Horvath clock-2)” and “PhenoAge”, the measurements were done in untreated (NT) and scramble (Scr) or LINE-1 (L1) antisense oligonucleotides (ASO) treated cells, P values were calculated using ratio paired parametric t-test. FIG. 3B shows a heatmap representation of RNA-seq differentially expressed genes analysis (FPKM fold change) in WT cells and HGPS cells untreated (NT) and scramble (Scr) or LINE-1 (L1) antisense oligonucleotides (ASO) treated, overexpressed genes are shown in yellow and downregulated genes in blue. FIG. 3C shows a heatmap representation of RNA-seq differentially expressed genes analysis (FPKM fold change) in WT cells and WRN cells untreated (NT) and scramble (Scr) or LINE-1 (L1) antisense oligonucleotides (ASO) treated, overexpressed genes are shown in yellow and downregulated genes in blue. FIG. 3D shows a pie chart showing the percentage of recovered genes in HGPS and WRN cells treated with (L1) antisense oligonucleotides (ASO). FIG. 3E shows a gene ontology (GO) analysis of RNA-seq data for upregulated and downregulated pathways associated with accelerated aging in L1 ASO versus Scr ASO-treated HGPS and WRN cells. Values represent the GO enrichment score. FIG. 3F shows a heatmap representation of differential protein stoichiometry analysis using mass spectrometry in WT cells and HGPS and WRN cells untreated (NT) and treated with scramble (Scr) or LINE-1 (L1) antisense oligonucleotides (ASO).

FIG. 4A shows a TaqMan qPCR analysis of LINE-I A, G, and T subfamilies in tail-tip fibroblasts (TTFs) isolated from WT (8 weeks and 24 months old) and LAKI (8 weeks old) mice. P values were calculated using multiple t-test with Holm-Sidak correction. FIG. 4B shows an immuno-FISH analysis of L1 RNA and progerin in WT and LAKI TTFs. Fluorescence signal quantification were performed on 100 nuclei for each replicate. Nuclei are counterstained with DAPI, scale bar, 5 μm, P values were calculated using unpaired t-test with Welch's correction. n=3. FIG. 4C shows on the left: an immuno-FISH analysis of LINE-1 RNA, H3K9me3, and H3K27me3 in WT and LAKI TTFs at passage 3 (early passage) and passage 8 (late passage); in the center: a fluorescence signal quantification were performed on 100 nuclei for each biological replicate, nuclei are counterstained with DAPI, scale bar, 5 μm, P values were calculated using unpaired t-test with Welch's correction, n=3; and on the right: an ELISA assay of H3K9me3 and H3K27me3 quantification on 10 μg of protein extracts. n=4, P values were calculated using multiple t-test with Holm-Sidak correction. FIG. 4D shows a qPCR analysis of SASP genes (p16, p21, Atf3, Gadd45b, Mmp13, Illa and BTG2) expression in WT and LAKI TTFs at passages 3 and 8, P values were calculated using multiple t-test with Holm-Sidak correction. FIG. 4E shows a senescence-associated β-galactosidase activity assay in LAKI TTFs at passage 3 and 8, P values were calculated using unpaired t-test with Welch's correction. FIG. 4F shows a TaqMan qPCR analysis of LINE-1 A, G, and T subfamilies and Major Satellite DNA (M-Sat) expression in WT and LAKI TTFs at early and late passages, P values were calculated using multiple t-test with Holm-Sidak correction. FIG. 4G shows a qPCR analysis of SUV39H1/2 RNA immunoprecipitation (fRIP) for 5′UTR-ORF1 and ORF2-3′UTR regions of LINE-1 RNA in passage 4 TTFs, P values were calculated using unpaired t-test with Welch's correction.

FIG. 5A shows a qPCR analysis of SASP genes (p16, p21, Atf3, Gadd45b, Mmp13, Il1a and BTG2) in WT and LAKI TTFs untreated and Scr or L1 ASO treated, P values were calculated with a multiple t-test with Holm-Sidak correction. FIG. 5B shows a senescence-associated β-galactosidase activity assay in LAKI TTFs treated with Scr or L1 ASOs. FIG. 5C shows an immuno-FISH analysis of LINE-1 RNA, H3K9me3, and H3K27me3 in WT and LAKI TTFs treated with Scr or L1 ASO. Fluorescence signal quantification was performed on 100 nuclei for each replicate, nuclei were counterstained with DAPI, scale bar, S m, P values were calculated using one-way ANOVA with Brown-Forsythe correction and unpaired t-test with Welch's correction, n=3.

FIG. 6A shows a schematic illustration of L1 and Scr ASO delivery in LAKI mice, 8-week old mice were treated with three intraperitoneal (IP) injections of 2 mg/kg ASO every ten days, at 16-weeks of age, mice were euthanized and tissues were collected. FIG. 6B shows survival curves of LAKI mice treated with Scr (Red) and L1 (blue) ASO. Non-injected mice were used as control (Black), P values were calculated with Mantel-Cox test (L1 ASO versus Scr ASO=0.0010. L1 ASO versus Control <0.0001. Scr ASO versus Control=0.0159) and Gehan-Breslow Test (L1 ASO versus Scr ASO=0.0056. L1 ASO versus Control <0.007. Scr ASO versus Control=0.0176). FIG. 6C shows a histological analysis of aorta (arch and thoracic trait), skin, kidney, and spleen of WT and LAKI mice treated with Scr or L1 ASO. Scale bar, 50 μm (aorta and kidney) and 100 μm (skin and spleen (left), quantification of aorta nuclear density, skin epidermal and dermal thickness, renal tubular atrophy and spleen germinal center diameters (right), P values were calculated using unpaired t-test with Welch's correction.

FIG. 7A shows an immuno-FISH analysis of LINE-1 RNA, H3K9me3, H3K27me3, and γH2AX in tissue sections of aorta (arch), skin, kidney, and spleen of WT and LAKI mice injected with Scr or L1 ASO, scale bar, 100 μm and 50 μm (spleen). FIG. 7B shows an ELISA quantification of H3K9me3, H3K27me3, and γH2AX in aorta, skin, kidney and spleen protein extract derived from WT and LAKI mice treated with Scr ASO or L1 ASO, 20 g of total protein extract was used as input, P values were calculated using multiple t-test with Holm-Sidak correction, n=4.

FIG. 8A shows a heatmap representation of RNA-seq differentially expressed genes analysis (FPKM fold change) in the aortic arch, thoracic aorta, skin, spleen and kidney of WT and LAKI mice treated with Scr ASO and L1 ASO, overexpressed genes are shown in yellow and downregulated genes in blue, n=3. FIG. 8B shows a gene ontology (GO) analysis of RNA-seq data for upregulated and downregulated pathways associated with progeria in L1 ASO versus Scr ASO-treated LAKI mice, values represent the GO enrichment score. FIG. 8C shows a pie chart showing the percentage of recovered genes in LAKI mice tissues treated with L1 ASO versus Scr ASO.

FIG. 9A shows a barplot showing Homo Sapiens specific (L1Hs) and Primate specific (L1P) LINE-1 elements expression in HGPS cells calculated using SQuIRE tool from RNA-seq data, n=3, data were filtered for FDR and P values lower than 0.05. FIG. 9B shows a barplot showing Homo Sapiens specific (L1Hs) and Primate specific (L1P) LINE-1 elements expression in WRN−/− cells calculated using SQuIRE tool from RNA-seq data, n=3, data were filtered for FDR and P values lower than 0.05. FIG. 9C shows LINE-1 ORF1 and ORF2 proteins stoichiometry after quantitative Mass Spectrometry analysis, n=3. FIG. 9D shows immunostaining of H3K9me3 and H3K27me3 histone H3 post-translational modifications and total histone H4 in WT, HGPS and WRN−/− cells, nuclei were counterstained with DAPI, scale bar, 10 μm.

FIG. 10A shows a time course immuno-FISH analysis of LINE-1 RNA, H3K9me3 and Progerin in Tet-On Progerin GFP inducible HDF cells, Doxycycline (1 g/ml) treated and untreated cells were collected at 12 hrs, 24 hrs, 2 days, 3 days and 4 days after Progerin GFP induction, fluorescence signal quantification were performed on 100 nuclei for each replicate, nuclei were counterstained with DAPI, scale bar, 10 μm, n=3. FIG. 10B shows a LINE-1, p16 and p21 qPCR expression analysis in GFP-Progerin cells and GFP-LaminA cells following doxycycline (doxo) treatment at different time points, P values were calculated using multiple t-Test with Holm-Sidak correction. FIG. 10C shows an immuno-FISH analysis of LINE-1 RNA in WT TTFs upon p16-HA and p21-FLAG overexpression. Cells transfected with an empty backbone were used as a control, scale bar, 20 μm. FIG. 10D shows a senescence-associated β-galactosidase activity assay in control and p16-HA/p21-FLAG overexpressing cells. FIG. 10E shows a TaqMan qPCR expression analysis of LINE-1 A, G, and T subfamilies in control and p16-HA/p21-FLAG overexpressing cells. FIG. 10F shows a LINE-1 enrichment analysis of SAMMY-seq datasets from Sebestyen et al. 2020, SQuIRE tool is used to measure LINE-1 elements enrichment in S2 (unbound DNA), S3 (DNA in the accessible chromatin compartment) and S4 (nuclear matrix and heterochromatin-associated DNA) fractions in control and HGPS cells, the heatmap represents the FPKM Fold Change value between fractions (blue-enriched in S4 and yellow in S3 or S2 fractions).

FIG. 11A shows a ChIP-qPCR showing H3K9me3 enrichment at different full-length LINE-1 loci. Data are expressed as a percentage of the input. FIG. 11B shows a barplot showing Homo Sapiens specific (L1Hs) and Primate specific (L1P) LINE-1 elements expression in HGPS cells untreated and treated with LINE-1 antisense oligonucleotide compared to WT, FPKM fold change values were calculated using SQuIRE tool from RNA-seq data, n=3, data were filtered for FDR and P values lower than 0.05. FIG. 11C shows a barplot showing Homo Sapiens specific (L1Hs) and Primate specific (L1P) LINE-1 elements expression in WRN−/− cells untreated and treated with LINE-1 antisense oligonucleotide compared to WT, FPKM fold change values were calculated using SQuIRE tool from RNA-seq data, n=3, data were filtered for FDR and P values lower than 0.05.

FIG. 12A shows a ChIP-qPCR showing H3K9me3 enrichment at different pericentromeric and intergenic loci in WT cells and HGPS cells untreated, LINE-1 (L1) ASO or scramble (scr) ASO treated, n=3, P values were calculated using multiple t-Test with Holm-Sidak correction. FIG. 12B shows a ChIP-qPCR showing H3K9me3 enrichment at different pericentromeric and intergenic loci in WT cells and WRN−/− cells untreated, LINE-1 (L1) ASO or scramble (scr) ASO treated, n=3, P values were calculated using multiple t-Test with Holm-Sidak correction.

FIG. 13A shows an immuno-FISH analysis of LINE-1 RNA and H4K20me3 in HGPS and WRN−/− hMSCs treated with Scr or L1-ASO, scale bar, 50 μm, nuclei ware counterstained with DAPI (left), immuno-FISH analysis of LINE-1 RNA, γH2AX, and Ki67 in HGPS and WRN−/− hMSCs treated with Scr or L1 ASO (right), scale bar, 50 μm, nuclei were counterstained with DAPI. FIG. 13B shows a qPCR analysis of senescence-associated gene expression in HGPS (top) and WRN−/− (bottom) hMSCs treated with mock, 3TC, LINE-1 translation blocker LNA gapmer (L1 T.B.) and scramble LNA gapmer, P values were calculated using multiple t-Test with Holm-Sidak correction. FIG. 13C shows an immuno-FISH analysis of LINE-1 RNA and H3K9me3 in HGPS and WRN−/− hMSCs treated with mock, 3TC, LINE-1 translation blocker LNA gapmer (L1 T.B.) or scramble LNA gapmer. Nuclei are counterstained with DAPI, scale bar, 50 μm. FIG. 13D shows a quantification of senescence-associated β-galactosidase positive HGPS and WRN−/− cells treated with mock, 3TC, LINE-1 translation blocker LNA gapmer (L1 T.B.) or scramble LNA gapmer.

FIG. 14A shows at the top an immuno-FISH analysis of LINE-1 RNA, H3K27me3 and H3K9me3 together with senescence-associated β-galactosidase assay in HGPS cells treated with scramble (scr) ASO or LINE-1 (L1) ASO upon SUV39H1 inhibition with Chaetocin (10 nM) or knockdown via shRNA. DMSO and empty shRNA vector was used as control, and at the bottom a senescence-associated β-galactosidase assay quantification, P values were calculated using multiple t-Test with Holm-Sidak correction, n=4, nuclei were counterstained with DAPI, scale bar, 10 μm. FIG. 14B shows on the left a qPCR analysis of SUV39H1 expression upon shRNA mediated knockdown, and on the right an ELISA quantification of H3K9me3 and H3K27me3 in HGPS cells treated with scramble ASO or LINE-1 ASO upon SUV39H1 inhibition with Chaetocin (10 nM) or knockdown via shRNA, DMSO and empty shRNA vector were used as control, 20 g of total protein extract was used as input, in orange, the ELISA assay positive control value, P values were calculated using multiple t-Test with Holm-Sidak correction, n=3. FIG. 14C shows a PCR analysis of senescence-associated gene expression in HGPS cells treated with scramble ASO or LINE-1 ASO upon SUV39H1 inhibition with Chaetocin (10 nM) or knockdown via shRNA. DMSO and empty shRNA vector were used as control. P values were calculated using multiple t-Test with Holm-Sidak correction, n=4. FIG. 14D shows at the top an immuno-FISH analysis of LINE-1 RNA, H3K27me3 and H3K9me3 together with senescence-associated β-galactosidase assay in HGPS cells treated with scramble ASO, LINE-1 ASO or scramble ASO with SUV39H1 overexpression, empty overexpression vector was used as control, and at the bottom a senescence-associated β-galactosidase assay quantification, p values were calculated using multiple t-Test with Holm-Sidak correction. n=4. Nuclei are counterstained with DAPI, scale bar, 10 μm. FIG. 14E shows a qPCR analysis of SUV39H1 overexpression. FIG. 14F shows a ELISA quantification of H3K9me3 and H3K27me3, in HGPS cells treated with scramble ASO, LINE-1 ASO or scramble ASO with SUV39H1 overexpression, empty vector was used as control. 20 g of total protein extract was used as input, in orange, the ELISA assay positive control value, p values were calculated using multiple t-Test with Holm-Sidak correction. n=4. FIG. 14G shows a qPCR analysis of senescence-associated gene expression in HGPS cells treated with scramble ASO, LINE-1 ASO or scramble ASO with SUV39H1 overexpression, empty vector was used as control, p values were calculated using multiple t-Test with Holm-Sidak correction, n=4.

FIG. 15A shows a qPCR analysis of senescence-associated gene p16 in WT and Progeroid syndromes driven by LMNA E578V, R644C, T10I and unknown mutation treated with Scr or L1 ASO, p values were calculated using multiple t-Test with Holm-Sidak correction. FIG. 15B shows a qPCR analysis of senescence-associated gene p21. FIG. 15C shows a qPCR analysis of senescence-associated gene ATF3. FIG. 15D shows a qPCR analysis of senescence-associated gene MMP13. FIG. 15E shows a qPCR analysis of senescence-associated gene BTG2. FIG. 15F shows a qPCR analysis of senescence-associated gene GADD45b.

FIG. 16A shows a TaqMan qPCR analysis of LINE-1 A, G, and T subfamilies in TTFs isolated from 8 weeks old LAKI mice and 24 months old WT mice untreated or after treatment with Scr or L1 ASO, p values were calculated using multiple t-Test with Holm-Sidak correction. FIG. 16B shows a RNA FISH analysis of L1 RNA in LAKI TTFs treated with Scr or L1 ASO, fluorescence signal quantification were performed on 100 nuclei for each biological replicate, scale bar, 5 μm, p value for FISH quantification were calculated using unpaired t-Test with Welch's correction. FIG. 16C shows an immuno-FISH analysis of L1 RNA, Progerin, γH2Ax and 53BP1 in LAKI TTFs treated with Scr or L1 ASO, nuclei were counterstained with DAPI, scale bar, 50 μm. FIG. 16D shows a quantification of Abnormal nuclei and γH2AX+ cells, the analysis was performed on 100 nuclei for each biological replicate. FIG. 16E shows an immuno-FISH analysis of L1 RNA, H3K9me3 and H3K27me3 in WT TTFs and WT TTFs with LINE-1spa element overexpression (L1spa), TTFs transfected with an empty vector were used as control. FIG. 16F shows on the left a s, nescence-associated β-galactosidase activity assay in WT TTFs with LINE-1spa element overexpression (L1spa) and TTFs with L1spa overexpression+L1 ASO treatment. TTFs transfected with an empty vector were used as control, and on the right a quantification of senescence-associated β-galactosidase positive cells in WT TTFs with LINE-1spa element overexpression (L1spa) and L1 ASO treatment. FIG. 16G shows on the left a TaqMan qPCR analysis of L1spa expression in WT TTFs with LINE-1spa element overexpression (L1spa) and TTFs with L1spa overexpression+L1 ASO treatment, TTFs transfected with an empty vector were used as control, p values were calculated using unpaired t-Test with Welch's correction, and on the right a qPCR analysis of senescence-associated genes expression in WT TTFs with L1 spa overexpression or L1spa overexpression+L1 ASO treatment. TTFs transfected with an empty vector were used as control. FIG. 16H shows an ELISA quantification of H3K9me3, H3K27me3, and γH2AX on nuclear protein extract from cells transfected with L1spa vector or an empty vector, 5 ag of nuclear protein was used as input, in orange, the ELISA assay positive control value.

FIG. 17A shows on the left an immuno-FISH analysis of LINE-1 RNA, H3K27me3 and H3K9me3 together with senescence-associated β-galactosidase assay in HGPS cells treated with scramble ASO, LINE-1 ASO or LINE-1 ASO with the overexpression of a mutated L1spa elements lacking the LINE-1 ASO target sequence (ΔL1spa), and on the right a quantification of senescence-associated β-galactosidase positive cells. Nuclei are counterstained with DAPI. Scale bar, 10 μm. FIG. 17B shows an ELISA quantification of H3K9me3 and H3K27me3 in HGPS cells treated with scramble ASO, LINE-1 ASO or LINE-1 ASO with ΔL1spa overexpression. In orange, the ELISA assay positive control value, n=4, p values were calculated using unpaired t-Test with Welch's correction. FIG. 17C shows a qPCR analysis of senescence-associated gene expression in HGPS cells treated with scramble ASO, LINE-1 ASO or LINE-1 ASO with ΔL1spa overexpression, n=4, p values were calculated using unpaired t-Test with Welch's correction.

FIG. 18A shows an IVIS imaging (Lumina series 3, Perkin Elmer) of fluorescently labeled L1 ASO injected mice, detection was done using Cy5 emission spectra 24 hrs and ten days after injection of ASO. FIG. 18B shows a RNA-seq analysis of L1 differential expression in the Aortic Arch, Thoracic Aorta, Skin, Spleen, Kidney and Skeletal muscle of WT and LAKI mice, the heatmap represents the fold change of the FPKM value for each L1 element subfamily in the mouse genome. FIG. 18C shows a RNA-seq analysis of L1 differential expression in the Aortic Arch, Thoracic Aorta, Skin, Spleen, Kidney, and Skeletal muscle of LAKI mice injected with Scr ASO or L1 ASO. The heatmap represents the fold change of the FPKM value for each L1 element subfamily in the mouse genome. FIG. 18D shows a bar plot showing the FPKM value of LINE-1 A, G, and T subfamilies in different LAKI mice tissues upon scr ASO or L1 ASO treatment compared to WT cells. FPKM values were calculated using SQuIRE tool for RNA-seq data analysis. (E) qPCR analysis of LINE-1 A, G, and T subfamilies in tissues of WT mice, LAKI mice untreated and LAKI mice treated with scr ASO or LINE-1 ASO. n=4.

FIG. 19 shows a Gene Set Enrichment Analysis of total RNA-seq. using the GSEA tool, the trend of DNA repair, senescence-associated p53 signaling, Cell Cycle, Heterochromatin organization, Pericentromeric Heterochromatin organization, and multicellular organism aging pathways in the Aorta (Arch and Thoracic), Skin, Spleen, and Kidney of WT and LAKI mice treated with Scr ASO or L1 ASO.

FIG. 20 shows L1Hs and L1P expression (FPKM Fold change) calculated by SQuIRE analysis of RNA-seq data of human cells from healthy donors of different ages, data were filtered for FDR and P values lower than 0.05.

### DETAILED DESCRIPTION OF THE INVENTION

In one aspect, the present invention relates to oligonucleotides, pharmaceutical compositions, and methods of inhibiting or decreasing human L1 retrotransposon RNAs. Such methods can be used in the treatment of progeria syndromes or signs and/or symptoms of aging. Aspects of the invention relate to administering a composition comprising an oligonucleotide described herein to a subject to treat, prevent, or inhibit a progeria syndrome or signs and/or symptoms of aging, or to rejuvenate tissue in a subject experiencing premature aging or having an age-related disease, or to reverse signs or symptoms of aging in a tissue of a subject.

### I. Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” can be understood to mean “at least one”; (ii) the term “or” can be understood to mean “and/or”; and (iii) the terms “including” and “comprising” can be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5% includes 5.0%, 5.1%, 5.18% without consideration of the number of significant figures).

As used herein, “no more than” or “less than” is understood as the value adjacent to the phrase and logical lower values or integers, as logical from context, to zero. For example, an oligonucleotide with “no more than 3 mismatches to a target sequence” has 3, 2, 1, or 0 mismatches to a target sequence. When “no more than” is present before a series of numbers or a range, it is understood that “no more than” can modify each of the numbers in the series or range.

As used herein, the term “administration” refers to the administration of a composition (e.g., a compound or a preparation that includes a compound as described herein) to a subject or system. Administration to a human can be by any appropriate route, such as one described herein.

As used herein, an “additional therapeutic agent” or “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some aspects, the delivery of the two or more agents is simultaneous or concurrent and the agents can be co-formulated. In some aspects, the two or more agents are not co-formulated and are administered in a sequential manner as part of a prescribed regimen. In some aspects, administration of two or more agents or treatments in combination is such that the reduction in a symptom, or other parameter related to a disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, intradermal route, subcutaneous route, intraperitoneal route, and direct absorption through mucous membrane tissues. The therapeutic agents can be administered by the same route or by different routes. For example, one therapeutic agent of the combination can be administered by intravenous injection while an additional therapeutic agent of the combination can be administered intradermally.

As used herein, the term “progeroid syndrome” refers to a disorder characterized by premature aging. Progeroid syndrome includes Hutchinson-Gilford progeria syndrome; Werner syndrome; mandibuloacral dysplasia type A; mandibuloacral dysplasia type B; mandibuloacral dysplasia associated to MTX2; mandibular hypoplasia, deafness, progeroid features and lipodystrophy syndrome (MDPL); Nestor—Guillermo progeria syndrome; and restrictive dermopathy. Mutations in a LMNA gene, ZMPSTE24 gene, BANF1 gene, POLD1 gene, MTX2 gene, or WRN gene are found in these progeria syndromes.

As used herein, the term “Hutchinson-Gilford progeria syndrome” or “HGPS” refers to an autosomal dominant genetic disorder in which symptoms resembling aspects of aging manifest at an early age. HGPS is characterized by de novo dominant point mutation (c.1824C>T; p. G608G) in the LMNA gene with onset in early childhood. The clinical features include severe failure to thrive in infancy, progressive alopecia leading to total alopecia, skin lesions, characteristic facies, loss of subcutaneous fat, bone changes, skeletal anomalies, musculoskeletal degeneration, hearing loss, high-pitched voice, delayed and crowded dentation, atherosclerosis, cerebrovascular disease, and death in mid teens from myocardial infarction or stroke.

As used herein “Werner syndrome” refers to a gentic disorder characterized by a mutation in the WRN gene with adult onset. The clinical features include lack of pubertal growth spurt during early teen years, graying or loss of hair, scleroderma-like skin lesions, characteristic facies, bilateral cataracts, type 2 diabetes mellitus, hypogonadism, skin ulcers, osteoporosis, atherosclerosis, and increased risk of cancer.

As used herein “mandibuloacral dysplasia type A” refers to a progeroid syndrome with early childhood onset that is caused by a recessive missense mutation in the LMNA gene. The clinical features include partial lipodystrophy at torso and limbs, bone abnormalities, altered skin pigmentation lipodystrophic signs and mildly accelerated aging.

As used herein “mandibuloacral dysplasia type B” refers to a progeroid syndrome with early childhood onset that is caused by a recessive mutations in the ZMPSTE24 gene with often compound heterozygous mutations with a null allele and one allele maintaining some residual activity. The clinical features include generalized lipodystrophy, altered skin pigmentation, alopecia, severe bone and growth defects.

As used herein “mandibuloacral dysplasia associated with MTX2” refers to a progeroid syndrome with early childhood onset that is caused by recessive mutations in the MTX2 gene. The clinical features include small viscerocranium with mandibular under development, growth retardation, lipodystrophy, altered skin pigmentation, distal acroosteolysis, renal focal glomerulosclerosis, and severe cardiovascular disease.

As used herein “restrictive dermopathy” refers to a progeroid syndrome with neonatal onset caused by recessive mutations in the ZMPSTE24 gene. The clinical features include intrauterine growth retardation, reduced fatal movements and preterm delivery, tight and translucent skin with erosions, skeletal malformations, generalized arthrogryposis, and death within the first few weeks of life.

As used herein “Nestor-Guillermo progenia syndrome” refers to a progeroid syndrome with early childhood onset caused a recessive mutation in the BANFI gene (c.34G>A; p.Ala12Thr). The clinical features include failure to thrive, aged appearance, growth retardation, decreased subcutaneous fat, thin limbs, stiff joints, severe osteolysis, absence of early cardiovascular impairment.

As used herein “mandibuloacral hypoplasia, progeroid features, and lipodystrophy syndrome” refers to a progeroid syndrome with early childhood onset that is caused by dominant mutations in the PODL1 gene including the common de novo mutation (c1812_1814delCTC, p.Ser605del). The clinical features include mandibular hypoplasia, prominent loss of subcutaneous fat, progeroid appearance, skin abnormalities, metabolic abnormalities including insulin resistance and diabetes mellitus, sensorineural deafness, and hypogonadism.

As used herein “atypical progeroid laminopathies” refer to progeroid syndromes with variable onset from early life to adulthood caused by mutations in the LMNA gene and ZMPSTE24 gene. The clinical features include symptoms ranging in severity from RD-like forms to adult-onset atypical WS. Atypical severe, MADB forms can result from recessive ZMPSTE24 mutations.

As used herein “disorders with nuclear envelope abnormalities” refer to disorders with mutations in the LMNA gene and include autosomal dominant (and rarely recessive) Emery-Dreifuss muscular dystrophy, Cardiomyopathy dilated 1A, Limb-girdle muscular dystrophy type 1i, congenital muscular dystrophy, “Heart-hand” syndrome, Dunnigan-type familiar partial lipodystrophy, Lipoatrophy with diabetes and other features of insulin resistance, atypical lipodystrophy syndromes, mandibular dysplasia, Charcot-Marie-Tooth type 2B1, Hutchinson-Gilford progeria syndrome, atypical Werner syndrome, and variant progerois disorders.

As used herein, the term “a subject identified as having signs and/or symptoms of aging” refers to a subject identified as having a molecular or pathological state, disease or condition of or associated with the aging process. The subject can have the signs and/or symptoms of aging at an age where the natural aging process would not cause such signs and/or symptoms. The subject can also have the signs and/or symptoms of aging at an age where the natural aging process would cause such signs and/or symptoms.

As used herein the terms “Long interspersed nuclear element”, “LINE-1,” or L-1 refer to retrotransposon elements whose expression is normally repressed in somatic cells by heterochromatin and whose re-expression is thought to affect genome stability, a hallmark of senescent and aging cells. Numerous human LINE-1 mRNA sequences have been identified and can be found at, for example, NCBI GenBank (see, e.g., www.ncbi.nlm.nih.gov). Non-limiting examples of LINE-1 can be found at, NCBI Accession Nos.: GU477636 (human) and AF016099.1 (mouse).

As used herein the term “antisense” refers to a nucleic acid comprising an oligonucleotide or polynucleotide that is sufficiently complementary to all or a portion of a gene, primary transcript, or processed mRNA, so as to interfere with expression of the endogenous gene (e.g., LINE-1), e.g., by inhibiting transcription of LINE-1 RNA or by decreasing stability of LINE-1 RNA.

As used herein, the term “target sequence” refers to a contiguous portion of the nucleotide sequence of an mRNA molecule formed during the transcription of a human LINE-1 gene, including mRNA that is a product of RNA processing of a primary transcription product. In one aspect, the target portion of the sequence will be at least long enough to serve as a substrate for oligonucleotide-directed (e.g., antisense oligonucleotide (ASO)-directed) cleavage at or near that portion of the nucleotide sequence of an mRNA molecule formed during the transcription of a human LINE-1 gene.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can refer to an alternative nucleotide, as further detailed below, or a surrogate replacement moiety. The oligonucleotide of SEQ ID NO: 1 is not limited to naturally occurring nucleosides but can contain non-natural nucleoside and linkages as disclosed herein, e.g., to produce an oligonucleotide that is modified to enhance its stability or cell permeability. The skilled person is well aware that guanine, cytosine, adenine, and uracil can be replaced by other moieties without substantially impairing the base-pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of oligonucleotides by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured herein.

The terms “nucleobase” and “base” include the purine (e.g. adenine and guanine) and pyrimidine (e.g. uracil, thymine, and cytosine) moiety present in nucleosides and nucleotides which form hydrogen bonds in nucleic acid hybridization. The term nucleobase also encompasses alternative nucleobases which can differ from naturally-occurring nucleobases, but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45 page 2055 and Bergstrom (2009) Current Protocols in Nucleic Acid Chemistry Suppl. 37 1.4.1.

Adenine and guanine are the two purine nucleobases most commonly found in nucleic acids. These may be substituted with other naturally-occurring purines, including but not limited to N⁶-methyladenine, N²-methylguanine, hypoxanthine, and 7-methylguanine. Cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligomers described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N²-cyclopentylguanine (cPent-G), N²-cyclopentyl-2-aminopurine (cPent-AP), and N²-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)).

Certain modified or substituted nucleobases are particularly useful for increasing the binding affinity of the antisense oligomers of the disclosure. These include 5-substituted pyrimidines, 6-azapyrimidines and N-2, N-6 and 0-6 substituted purines, including 2-aminopropyladenine, 5-propynyluracil, 5-propynylcytosine, and 5-methylcytosin

The term “nucleoside” refers to a monomeric unit of an oligonucleotide or a polynucleotide having a nucleobase and a sugar moiety. A nucleoside can include those that are naturally-occurring as well as alternative nucleosides, such as those described herein. The nucleobase of a nucleoside can be a naturally-occurring nucleobase or an alternative nucleobase. Similarly, the sugar moiety of a nucleoside can be a naturally-occurring sugar or an alternative sugar.

The term “alternative nucleoside” refers to a nucleoside having an alternative sugar or an alternative nucleobase, such as those described herein.

In some aspects the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2-thio-thymine, inosine, diaminopurine, 6-aminopurine, 2-aminopurine, 2,6-diaminopurine, and 2-chloro-6-aminopurine.

The nucleobase moieties can be indicated by the letter code for each corresponding nucleobase, e.g. A, T, G, C, or U, wherein each letter can include alternative nucleobases of equivalent function. In some aspects, e.g., for gapmers, a modified nucleoside can be used. In some aspects, a 2′ FANA modified nucleoside can be used.

A “sugar” or “sugar moiety,” includes naturally occurring sugars having a furanose ring. A sugar also includes an “alternative sugar,” defined as a structure that is capable of replacing the furanose ring of a nucleoside. In some aspects, alternative sugars are non-furanose (or 4′-substituted furanose) rings or ring systems or open systems. Alternative sugars can include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization. In some aspects, an oligonucleotide contains nucleosides with, e.g., 2′FANA modification and nucleosides without 2′ FANA modification, which nucleosides may contain alternative sugars as describe herein.

As used herein “2′ FANA,” refers to a 2′deoxy-2′fluoro-β-D arabinonuclic acid sugar modification in an antisense oligonucleotide, and an oligonucleotide that includes 2′ FANA is a “2′ FANA modified” oligonucleotide. 2′ FANA modified oligonucletoides form stable, nuclease resistant oligonucleotide/RNA duplexes and support ribonuclease H-mediated heteroduplex cleavage. 2′ FANA modified oligonucleotides also maintain high intracellular concentrations for prolonged periods compared to unmodified oligonucleotides.

A “nucleotide,” as used herein, refers to a monomeric unit of an oligonucleotide or polynucleotide that comprises a nucleoside and an internucleosidic linkage. The internucleosidic linkage can include a phosphate linkage. Similarly, “linked nucleosides” can be linked by phosphate linkages. Many “alternative internucleosidic linkages” or “non-natural linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which can include alternative nucleoside linkages.

The terms “oligonucleotide” and “polynucleotide,” as used herein, are defined as it is generally understood by the skilled person as a molecule comprising two or more covalently linked nucleosides. Such covalently bound nucleosides can be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide can be man-made. For example, the oligonucleotide can be chemically synthesized, and be purified or isolated. Oligonucleotide is also intended to include (i) compounds that have one or more 2′ FANA modified nucleosides, (ii) compounds that have one or more furanose moieties that are replaced by furanose derivatives or by any structure, cyclic or acyclic, that can be used as a point of covalent attachment for the base moiety, (iii) compounds that have one or more phosphodiester linkages that are either modified, as in the case of phosphoramidate or phosphorothioate linkages, or completely replaced by a suitable linking moiety as in the case of formacetal or riboacetal linkages, and/or (iv) compounds that have one or more linked furanose-phosphodiester linkage moieties replaced by any structure, cyclic or acyclic, that can be used as a point of covalent attachment for the base moiety. The oligonucleotide can comprise one or more alternative nucleosides or nucleotides (e.g., including those described herein). It is also understood that oligonucleotide includes compositions lacking a sugar moiety or nucleobase but are still capable of forming a pairing with or hybridizing to a target sequence. As used herein “oligonucleotide” refers to a short polynucleotide (e.g., of 100 or fewer linked nucleosides). “Chimeric” oligonucleotides or “chimeras,” as used herein, are oligonucleotides which contain two or more chemically distinct regions, each made up of at least one monomer unit, i.e., a nucleotide or nucleoside in the case of an oligonucleotide. Chimeric oligonucleotides also include “gapmers.” For example, chimeric oligonucleotides can contain unmodified nucleosides and 2′ FANA modified nucleosides. The oligonucleotides of the invention include at least one 2′ FANA modified nucleoside. In some aspects, the 2′ FANA modified nucleosides are located at a 5′ or a 3′ portion, or both, of an oligonucleotide. In some aspects, the 2′ FANA modified nucleosides are located throughout an oligonucleotide. In some aspects, a chimeric oligonucleotide comprises 2′ FANA modified nucleosides located at a 5′ and/or a 3′ portion and at least one unmodified nucleoside in the center of the oligonucleotide.

The term “gapmer,” as used herein, refers to an oligonucleotide which comprises a region of RNase H recruiting oligonucleotides (gap or DNA core) which is flanked 5′ and 3′ by regions which comprise one or more affinity enhancing alternative nucleosides (wings or flanking sequence). Various gapmer designs are described herein. Headmers and tailmers are oligonucleotides capable of recruiting RNase H where one of the flanks is missing, i.e. only one of the ends of the oligonucleotide comprises affinity enhancing alternative nucleosides. For headmers the 3′ flanking sequence is missing (i.e. the 5′ flanking sequence comprises affinity enhancing alternative nucleosides) and for tailmers the 5′ flanking sequence is missing (i.e. the 3′ flanking sequence comprises affinity enhancing alternative nucleosides). A “mixed flanking sequence gapmer” refers to a gapmer wherein the flanking sequences comprise at least one alternative nucleoside, such as at least one DNA nucleoside or at least one 2′ substituted alternative nucleoside, such as, for example, 2′-Fluoro-arabinonucleic acid nucleoside, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, or bicyclic nucleosides (e.g., locked nucleosides or constrained ethyl (cEt) nucleosides). In some aspects the mixed flanking sequence gapmer has one flanking sequence which comprises alternative nucleosides (e.g. 5′ or 3′) and the other flanking sequence (3′ or 5′ respectfully) comprises 2′ substituted alternative nucleoside(s).

As used herein, the term “oligonucleotide comprising a nucleobase sequence” refers to an oligonucleotide comprising a chain of nucleotides or nucleosides that is described by the sequence referred to using the standard nucleotide nomenclature.

The term “contiguous nucleobase region” refers to the region of the oligonucleotide which is complementary to the target nucleic acid. The term can be used interchangeably herein with the term “contiguous nucleotide sequence” or “contiguous nucleobase sequence.” In some aspects all the nucleotides of the oligonucleotide are present in the contiguous nucleotide or nucleoside region. In some aspects the oligonucleotide comprises the contiguous nucleotide region and can comprise further nucleotide(s) or nucleoside(s). For example, a nucleotide linker region which can be used to attach a functional group to the contiguous nucleotide sequence. The nucleotide linker region can be complementary to the target nucleic acid. In some aspects the internucleoside linkages present between the nucleotides of the contiguous nucleotide region are all phosphorothioate internucleoside linkages. In some aspects, the contiguous nucleotide region comprises one or more sugar-modified nucleosides.

A “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some aspects the conjugate or oligonucleotide conjugate can, comprise a linker region which is positioned between the oligonucleotide and the conjugate moiety. In some aspects, the linker between the conjugate and oligonucleotide is biocleavable. Phosphodiester containing biocleavable linkers are described in more detail in WO 2014/076195 (herein incorporated by reference).

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides or nucleosides. Complementary sequences can include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein.

As used herein, an “agent that reduces the level and/or activity of human LINE-1” refers to any polynucleotide agent (e.g., an oligonucleotide, e.g., an ASO) that reduces the level of human LINE-1 or inhibits expression of human LINE-1 in a cell or subject. The phrase “inhibiting expression of LINE-1,” as used herein, includes inhibiting expression of a human LINE-1 mRNA (e.g., ORF1, ORF2, and/or a protein encoded therein) or variants or mutants of a LINE-1 mRNA that encode LINE-1 proteins. Thus, the LINE-1 mRNA can be from a wild-type LINE-1 ORF, a mutant LINE-1 ORF, or a transgenic LINE-1 ORF in the context of a genetically manipulated cell, group of cells, or organism.

By “reducing the activity of LINE-1” is meant decreasing the level of an activity related to LINE-1 (e.g., by reducing the level of LINE-1 mRNA expression). The activity level of LINE-1 can be measured using any method known in the art (e.g., by directly measuring reverse transcriptase activity of the LINE-1 encoded reverse transcriptase).

By “reducing the level of LINE-1,” is meant decreasing the level of LINE-1 in a cell or subject, e.g., by administering an oligonucleotide to the cell or subject. The level of LINE-1 can be measured using any method known in the art (e.g., by measuring the level(s) of LINE-1 mRNA or level(s) of LINE-1 encoded protein(s) in a cell or a subject).

The term “inhibiting,” as used herein, is used interchangeably with “reducing,” “silencing,” “downregulating,” “suppressing,” and other similar terms, and includes any level of inhibition.

The phrase “contacting a cell with an oligonucleotide,” such as an oligonucleotide, as used herein, includes contacting a cell by any possible means. Contacting a cell with an oligonucleotide includes contacting a cell in vitro with the oligonucleotide or contacting a cell in vivo with the oligonucleotide. The contacting can be done directly or indirectly. Thus, for example, the oligonucleotide can be put into physical contact with the cell by the individual performing the method, or alternatively, the oligonucleotide agent can be put into a situation that will permit or cause it to subsequently come into contact with the cell.

Contacting a cell in vitro can be done, for example, by incubating the cell with the oligonucleotide. Contacting a cell in vivo can be done, for example, by injecting the oligonucleotide into or near the tissue where the cell is located, or by injecting the oligonucleotide agent into another area, e.g., the bloodstream or the subcutaneous space, such that the agent will subsequently reach the tissue where the cell to be contacted is located. For example, the oligonucleotide can contain and/or be coupled to a ligand that directs the oligonucleotide to a site of interest, e.g., the aorta. Combinations of in vitro and in vivo methods of contacting are also possible. For example, a cell can be contacted in vitro with an oligonucleotide and subsequently transplanted into a subject.

In one aspect, contacting a cell with an oligonucleotide includes “introducing” or “delivering the oligonucleotide into the cell” by facilitating or effecting uptake or absorption into the cell. Absorption or uptake of an ASO can occur through unaided diffusive or active cellular processes, or by auxiliary agents or devices. Introducing an oligonucleotide into a cell can be in vitro and/or in vivo. For example, for in vivo introduction, oligonucleotides can be injected into a tissue site or administered systemically. In vitro introduction into a cell includes methods known in the art such as electroporation and lipofection. Further approaches are described herein below and/or are known in the art.

As used herein, the term “vector” is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. In some aspects, such vectors include, but are not limited to: an adenoviral vector, an adeno-associated virus (AAV) vector, retroviral vector, a lentiviral vector, poxvirus vector, a baculovirus vector, a herpes viral vector, simian virus 40 (SV40), cytomegalovirus (CMV), mouse mammary tumor virus (MMTV), and Moloney murine leukemia virus. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can sometimes be used interchangeably, depending on the context, as the plasmid is the most commonly used form of vector. However, also disclosed herein are other forms of expression vectors, such as viral vectors (e.g., replication defective retroviruses, poxviruses, herpesviruses, baculoviruses, adenoviruses and adeno-associated viruses), which can serve equivalent functions.

As used herein, “lipid nanoparticle” or “LNP” is a vesicle comprising a lipid layer encapsulating a pharmaceutically active molecule, such as a nucleic acid molecule, e.g., an oligonucleotide. LNP refers to a stable nucleic acid-lipid particle. LNPs typically contain a cationic lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it can. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

As used herein, the terms “effective amount,” “therapeutically effective amount,” and “a “sufficient amount” of an agent that reduces the level and/or activity of LINE-1 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a progeria syndrome, it is an amount of the agent that reduces the level and/or activity of LINE-1 sufficient to achieve a treatment response as compared to the response obtained without administration of the agent that reduces the level and/or activity of LINE-1. The amount of a given agent that reduces the level and/or activity of LINE-1 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of LINE-1 of the present disclosure is an amount which results in a beneficial or desired result in a subject as compared to a control. As defined herein, a therapeutically effective amount of an agent that reduces the level and/or activity of LINE-1 of the present disclosure can be readily determined by one of ordinary skill by routine methods known in the art. Dosage regimen can be adjusted to provide the optimum therapeutic response.

“Prophylactically effective amount,” as used herein, is intended to include the amount of an oligonucleotide that, when administered to a subject aging prematurely or predisposed to prematurely age or predisposed to have a progeria syndrome, is sufficient to prevent or ameliorate the disease or one or more symptoms of the disease. Ameliorating the disease includes slowing the course of the disease or reducing the severity of later-developing disease. The “prophylactically effective amount” can vary depending on the oligonucleotide, how the agent is administered, the degree of risk of disease, and the history, age, weight, family history, genetic makeup, the types of preceding or concomitant treatments, if any, and other individual characteristics of the patient to be treated. A prophylactically effective amount can refer to, for example, an amount of the agent that reduces the level and/or activity of LINE-1 (e.g., in a cell or a subject) described herein or can refer to a quantity sufficient to, when administered to the subject, including a human, delay the onset of one or more of the premature aging or the signs and symptoms of natural aging described herein by at least 120 days, for example, at least 6 months, at least 12 months, at least 2 years, at least 3 years, at least 4 years, at least 5 years, at least 10 years or more, when compared with the predicted onset.

A “therapeutically-effective amount” or “prophylactically effective amount” also includes an amount (either administered in a single or in multiple doses) of an oligonucleotide that produces some desired local or systemic effect at a reasonable benefit/risk ratio applicable to any treatment. Oligonucleotides employed in the methods herein can be administered in a sufficient amount to produce a reasonable benefit/risk ratio applicable to such treatment.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps (DNA core sequences), if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “level” is meant a level or activity of a protein, or mRNA encoding one or more proteins (e.g., a LINE-1 mRNA), optionally as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein can be expressed in mass/vol (e.g., g/dL, mg/mL, g/mL, or ng/mL) or percentage relative to total protein or mRNA in a sample.

The term “pharmaceutical composition,” as used herein, represents a composition containing a compound described herein formulated with a pharmaceutically acceptable excipient, and can be manufactured or sold with the approval of a governmental regulatory agency as part of a therapeutic regimen for the treatment of disease in a subject. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein can have ionizable groups so as to be capable of preparation as pharmaceutically acceptable salts. These salts can be acid addition salts involving inorganic or organic acids or the salts can, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts can be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a young healthy subject, such as a normal cell or normal tissue; a sample (e.g., a cell or tissue) from a subject not having any signs and/or symptoms of aging; a sample from a subject that is diagnosed with a disease, but not yet treated with a compound described herein; a sample from a subject that is aging or aging prematurely, but not yet treated with a compound described herein a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder or a young subject or a subject not having signs and/or symptoms of aging; a subject that has been treated with a compound described herein. In some aspects, the reference sample, standard, or level is matched to the sample subject sample by at least one of the following criteria: age, weight, sex, disease stage, and overall health. A standard curve of levels of a purified protein, e.g., any described herein, within the normal reference range can be used as a reference.

As used herein, the term “subject” refers to a human. The terms “subject” and “patient” are used interchangeably herein.

As used herein, the terms “treat,” “treated,” and “treating” mean therapeutic treatment wherein the object is to reduce or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein the terms “preventing,” and “inhibiting” refer to prophylactic or preventative measures, the object of which is to prevent an undesired physiological condition, disorder, or disease from manifesting itself.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein can retain or improve upon the biological activity of the original material.

Various aspects of the invention are described in further detail in the following subsections.

### II. Antisense Oligonucleotides

Agents described herein that reduce the level and/or activity of LINE-1 in a cell can be antisense oligonucleotides. These agents reduce the level of LINE-1 in a cell or subject.

In some aspects, the agent that reduces the level and/or activity of LINE-1 is a polynucleotide. In some aspects, the polynucleotide is an oligonucleotide. In some aspects, the polynucleotide is a single-stranded oligonucleotide, e.g., that acts by way of an RNase H-mediated pathway. Oligonucleotides include DNA and DNA/RNA chimeric molecules which recognize polynucleotide target sequences or sequence portions through hydrogen bonding interactions with the nucleotide bases of the target sequence (e.g., LINE-1). An oligonucleotide molecule can decrease the expression level (e.g., protein level or mRNA level) of LINE-1. The oligonucleotides of the invention decrease the level and/or activity or function of LINE-1. The oligonucleotide inhibits expression of LINE-1. In some aspects, the oligonucleotide increases degradation of LINE-1 mRNA and/or decreases the stability (i.e., half-life) of LINE-1 mRNA. The oligonucleotide can be chemically synthesized.

The oligonucleotide has a region of complementarity (e.g., a contiguous nucleobase region) that is complementary to a part of an mRNA formed in the expression of a LINE-1 retrotransposon. Upon contact with a cell expressing the LINE-1 gene, the oligonucleotide can inhibit the expression of the LINE-1 retrotransposon, e.g., by RNAse digestion of the LINE-1 mRNA by at least about 10% as assayed by, for example, a PCR or branched DNA (bDNA)-based method, or by a protein-based method, such as by immunofluorescence analysis, using, for example, Western Blotting or flowcytometric techniques.

An oligonucleotide can be synthesized by standard methods known in the art as further discussed below, e.g., by use of an automated DNA synthesizer, such as are commercially available from, for example, Biosearch, Applied Biosystems, Inc.

The oligonucleotide compound can be prepared using solution-phase or solid-phase organic synthesis or both. Organic synthesis offers the advantage that the oligonucleotide comprising unnatural or alternative nucleotides can be easily prepared. Single-stranded oligonucleotides can be prepared using solution-phase or solid-phase organic synthesis or both. In some aspects, single-stranded oligonucleotides can be hybridized to a nucleic acid before use in a method as described herein. For example, single-stranded DNA oligonucleotides can be hybridized to RNA molecules and DNA/RNA complexes can be used in the methods described herein. In some aspects, single-stranded oligonucleotides are used in the methods described herein.

The sequence of the oligonucleotide is SEQ ID NO: 1, with at least one 2′ FANA modification, but may have additional modified sugars, alternative bases, non-natural linkages, or other modifications as described herein. In some aspects the oligonucleotide, or a contiguous nucleotide region thereof, has a gapmer design or structure also referred herein merely as a “gapmer.” In a gapmer structure the oligonucleotide comprises at least three distinct structural regions a 5′-flanking sequence (also known as a 5′-wing), a central portion or DNA core sequence (also known as a gap) and a 3′-flanking sequence (also known as a 3′-wing), in ‘5->3’ orientation. In this design, the 5′ and 3′ flanking sequences comprise at least one alternative nucleoside which is adjacent to a DNA core sequence, and can, in some aspects, comprise a contiguous stretch of 2-7 alternative nucleosides, or a contiguous stretch of alternative and DNA nucleosides (mixed flanking sequences comprising both alternative and DNA nucleosides).

The length of the 5′-flanking sequence region can be at least two nucleosides in length (e.g., at least at least 2, at least 3, at least 4, at least 5, or more nucleosides in length). The length of the 3′-flanking sequence region can be at least two nucleosides in length (e.g., at least 2, at least 3, at least at least 4, at least 5, or more nucleosides in length). The 5′ and 3′ flanking sequences can be symmetrical or asymmetrical with respect to the number of nucleosides they comprise. In some aspects, the DNA core sequence comprises about 10 nucleosides flanked by a 5′ and a 3′ flanking sequence each comprising about 5 nucleosides, also referred to as a 5-10-5 gapmer.

In some aspects, the nucleosides of the 5′ flanking sequence and the 3′ flanking sequence which are adjacent to the DNA core sequence are alternative nucleosides, such as 2′ alternative nucleosides. The DNA core sequence comprises a contiguous stretch of nucleotides which are capable of recruiting RNase H, when the oligonucleotide is in duplex with the LINE-1 target nucleic acid. In some aspects, the DNA core sequence comprises a contiguous stretch of 5-16 DNA nucleosides.

The 5′ and 3′ flanking sequences, flanking the 5′ and 3′ ends of the DNA core sequence, can comprise one or more affinity enhancing alternative nucleosides. In some aspects, the 5′ and/or 3′ flanking sequence comprises at least one 2′ fluoroarabinonucleic acid (2′ FANA) nucleoside. In some aspects, the 5′ and/or 3′ flanking sequence comprises at least one 2′-O-methoxyethyl (MOE) nucleoside. In some aspects, the 5′ and/or 3′ flanking sequences, contain at least one 2′ FANA and at least one MOE nucleoside. In some aspects, the 5′ flanking sequence comprises at least one 2′ FANA nucleoside. In some aspects, the 3′ flanking sequence comprises at least one 2′ FANA nucleoside. In some aspects both the 5′ and 3′ flanking sequence comprise a 2′ FANA nucleoside. In some aspects, all the nucleosides in the flanking sequences are 2′ FANA nucleosides. In other aspects, the flanking sequence can comprise both 2′ FANA nucleosides and other nucleosides (mixed flanking sequence), such as DNA nucleosides and/or non-2′ FANA alternative nucleosides, such as bicyclic nucleosides (BNAs) (e.g., LNA nucleosides or cET nucleosides), or other 2′ substituted nucleosides including 2′-O-methoxyethyl (MOE) nucleoside. In some aspects, the DNA core sequence is defined as a contiguous sequence of at least 5 RNase H recruiting nucleosides (such as 5-16 DNA nucleosides) flanked at the 5′ and 3′ end by 2′ FANA nucleosides.

The 5′ flanking sequence attached to the 5′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three or four alternative nucleobases. In some aspects, the flanking sequence comprises or consists of at least one alternative internucleoside linkage (e.g., at least three, at least four, at least five, at least six, at least seven, or more alternative internucleoside linkages).

The 3′ flanking sequence attached to the 3′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three, or four alternative nucleobases. In some aspects, the flanking sequence comprises or consists of at least one alternative internucleoside linkage (e.g., at least three, at least four, at least five, at least six, at least seven, or more alternative internucleoside linkages).

In some aspects, one or more or all of the alternative sugar moieties in the flanking sequence are 2′ alternative sugar moieties.

In a further aspect, one or more of the 2′ alternative sugar moieties in the flanking sequences are selected from 2′-FANA sugar moieties, 2′-O-alkyl-sugar moieties, 2′-O-methyl-sugar moieties, 2′-amino-sugar moieties, 2′-fluoro-sugar moieties, 2′-alkoxy-sugar moieties, MOE sugar moieties, LNA sugar moieties, and arabino nucleic acid (ANA) sugar moieties.

In some aspects, all the alternative nucleosides in the flanking sequences are 2′ FANA nucleosides. In some aspects, some of the alternative nucleosides in the flanking sequences are 2′ FANA nucleosides and some are 2′-O-alkyl-sugar moieties, 2′-O-methyl-sugar moieties, 2′-amino-sugar moieties, 2′-alkoxy-sugar moieties, MOE sugar moieties, LNA sugar moieties, or arabino nucleic acid (ANA) sugar moieties. In some aspects, the bicyclic nucleosides in the flanking sequences are independently selected from the group consisting of oxy-LNA, thio-LNA, amino-LNA, cET, and/or ENA, in either the beta-D or alpha-L configurations or combinations thereof.

In some aspects, an oligonucleotide includes nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytidine, 5-hydroxymethylcytidine, 5-formylcytidine, 5-carboxycytidine, pyrrolocytidine, dideoxycytidine, uridine, 5-methoxyuridine, 5-hydroxydeoxyuridine, dihydrouridine, 4-thiourdine, pseudouridine, 1-methyl-pseudouridine, deoxyuridine, 5-hydroxybutynl-2′-deoxyuridine, xanthine, hypoxanthine, 7-deaza-xanthine, thienoguanine, 8-aza-7-deazaguanosine, 7-methylguanosine, 7-deazaguanosine, 6-aminomethyl-7-deazaguanosine, 8-aminoguanine, 2,2,7-trimethylguanosine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouridine, 2-thiothymine and 2-thiocytosine, 5-halouracil and cytosine, 5-propynyl uridine and cytidine, 6-azo uridine, cytidine and thymine, 4-thiouridine, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uridines and cytidines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligonucleotide. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

In some aspects, the sugar moiety in the nucleotide can be a ribose molecule, having a 2′-FANA and a 2′-O-methyl, 2′-O-MOE, 2′-F, 2′-amino, 2′-O-propyl, 2′-aminopropyl, or 2′-OH modification.

In some aspects, an oligonucleotide can include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In some aspects, the bridge connects the 4′-carbon and the 2′-carbon of the sugar ring. In some aspects, an oligonucleotide can include one or more locked nucleosides. A locked nucleoside is a nucleoside having a modified ribose moiety in which the ribose moiety comprises an extra bridge connecting the 2′ and 4′ carbons. In other words, a locked nucleoside is a nucleoside comprising a bicyclic sugar moiety comprising a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the polynucleotides include without limitation nucleosides comprising a bridge between the 4′ and the 2′ ribosyl ring atoms. In some aspects, the oligonucleotides include one or more bicyclic nucleosides comprising a 4′ to 2′ bridge. In some aspects, the oligonucleotides include one or more 2′ FANA nucleoside and one or more bicyclic nucleosides. Examples of 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂₋₂′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Any of the foregoing bicyclic nucleosides can be prepared having one or more stereochemical sugar configurations including for example α-L-ribofuranose and 3-D-ribofuranose (see WO 99/14226).

An oligonucleotide can be modified to include one or more constrained ethyl nucleosides. As used herein, a “constrained ethyl nucleoside” or “cEt” is a locked nucleoside comprising a bicyclic sugar moiety comprising a 4′-CH(CH₃)—O-2′ bridge. In one aspect, a constrained ethyl nucleoside is in the S conformation referred to herein as “S-cEt.”

An oligonucleotide can include one or more “conformationally restricted nucleosides” (“CRN”). CRN are nucleoside analogs with a linker connecting the C2′ and C4′ carbons of ribose or the C3 and --C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering. Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some aspects, an oligonucleotide comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule can be modified with a cyclopropane ring to produce a tricyclodeoxynucleic acid (tricyclo DNA). The ribose moiety can be substituted for another sugar such as 1,5,-anhydrohexitol, threose to produce a threose nucleoside (TNA), or arabinose to produce an arabino nucleoside. The ribose molecule can be replaced with non-sugars such as cyclohexene to produce cyclohexene nucleoside or glycol to produce glycol nucleosides.

In some aspects, potentially stabilizing modifications at the ends of nucleoside molecules include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

In some aspects, alternative chemistries of an oligonucleotide include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

In some aspects, oligonucleotides comprise nucleosides with alternative sugar moieties and DNA or RNA nucleosides. In some aspects, the oligonucleotide comprises nucleosides comprising alternative sugar moieties and DNA nucleosides. Incorporation of alternative nucleosides into the oligonucleotide can enhance the affinity of the oligonucleotide for the target nucleic acid. In that case, the alternative nucleosides can be referred to as affinity enhancing alternative nucleotides.

In some aspects, the oligonucleotide comprises at least 1 alternative nucleoside, such as at least 2, at least 3, at least 4, at least 5, at least 6, at least 7, at least 8, at least 9, at least 10, at least 11, at least 12, at least 13, at least 14, at least 15, at least 16, at least 18, at least 20, or at least 21 alternative nucleosides. In some aspects, the oligonucleotides comprise from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In some aspects, the oligonucleotide comprise alternatives, which are independently selected from these three types of alternatives (alternative sugar moiety, alternative nucleobase, and alternative internucleoside linkage), or a combination thereof. In some aspects, the oligonucleotide comprises one or more nucleosides comprising alternative sugar moieties, e.g., 2′ sugar alternative nucleosides. In some aspect, the oligonucleotide comprises the one or more 2′ sugar alternative nucleoside independently selected from the group consisting of 2′-fluoro-ANA, 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), and BNA (e.g., LNA) nucleosides. In some aspects, the one or more alternative nucleoside is a 2′ FANA.

At least 1 of the alternative nucleosides is a 2′ FANA. In some aspects, at least 2, such as at least 3, at least 4, at least 5, at least 6, at least 7, or at least 8 of the alternative nucleosides are 2′ FANAs. In a still further aspect, all the alternative nucleosides are 2′ FANAs.

In some aspects, the one or more alternative internucleoside linkages in the flanking sequences are phosphorothioate internucleoside linkages. In some aspects, the phosphorothioate linkages are stereochemically pure phosphorothioate linkages. In some aspects, the phosphorothioate linkages are Sp phosphorothioate linkages. In other aspects, the phosphorothioate linkages are Rp phosphorothioate linkages. In some aspects, the alternative internucleoside linkages are 2′-alkoxy internucleoside linkages. In other aspects, the alternative internucleoside linkages are alkyl phosphate internucleoside linkages.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included. The preparation of the above phosphorus-containing linkages is known in the art and disclosed, e.g., in U.S. Pat. Nos. 6,878,805; 7,015,315; 7,041,816; 7,273,933; 7,321,029, each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. The preparation of the alternative internucleoside linkages is known in the art and disclosed, e.g., in U.S. Pat. Nos. 5,623,070; 5,663,312; 5,633,360; 5,677,437; and, 5,677,439, each of which are hereby incorporated herein by reference.

In some aspects, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. The preparation of PNAs is known in the art and disclosed, e.g., in U.S. Pat. Nos. 5,539,082; 5,714,331; and 5,719,262, each of which are hereby incorporated herein by reference. Additional PNA compounds suitable for use in the oligonucleotides are described in, for example, Nielsen et al., Science, 1991, 254, 1497-1500.

In some aspects, oligonucleotides comprise phosphorothioate backbones and oligonucleotides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂—]. In some aspects, the oligonucleotides have morpholino backbone structures. In some aspects, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70. In some aspects, the bases of the oligonucleotide are linked to morpholino ring structures, wherein the morpholino ring structures are joined by phosphorous-containing intersubunit linkages joining a morpholino nitrogen of one ring structure to a 5′ exocyclic carbon of an adjacent ring structure. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, and 8,299,206, all of which are incorporated herein by reference.

In some aspects, the oligonucleotides contain a 2′ FANA modified nucleoside and, optionally further modified oligomers including morpholino oligomers, phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate nucleotides, 2′-O-[2-(N-methylcarbamoyl)ethyl]modified oligomers, including combinations of any of the foregoing. In some aspects, 2′ FANA modified oligonucleotides are combined with alternative nucleotides having phosphorothioate and 2′-O-Me-modified chemistries to generate a 2′O-Me-phosphorothioate backbone.

In some aspects, all of the nucleosides of the DNA core sequence are DNA units. In some aspects, the DNA core region can consist of a mixture of DNA and other nucleosides capable of mediating RNase H cleavage. In some aspects, at least 50% of the nucleosides of the DNA core sequence are DNA, such as at least 60%, at least 70% or at least 80%, or at least 90% DNA. In some aspects, all of the nucleosides of the DNA core sequence are RNA units.

In some aspects, the oligonucleotide comprises a contiguous region which is complementary to the target nucleic acid. In some aspects, the oligonucleotide can further comprise additional linked nucleosides positioned 5′ and/or 3′ to either the 5′ and 3′ flanking sequences. These additional linked nucleosides can be attached to the 5′ end of the 5′ flanking sequence or the 3′ end of the 3′ flanking sequence, respectively. The additional nucleosides can, in some aspects, form part of the contiguous sequence which is complementary to the target nucleic acid, or in other aspects, can be non-complementary to the target nucleic acid.

In some aspects, the inclusion of the additional nucleosides at either, or both of the 5′ and 3′ flanking sequences can independently comprise one, two, three, four, or five additional nucleotides, which can be complementary or non-complementary to the target nucleic acid. In some aspects, the oligonucleotide can comprise a contiguous sequence capable of modulating the target which is flanked at the 5′ and/or 3′ end by additional nucleotides. Such additional nucleosides can serve as a nuclease susceptible biocleavable linker, and can therefore be used to attach a functional group such as a conjugate moiety to the oligonucleotide. In some aspects, the additional 5′ and/or 3′ end nucleosides are linked with phosphodiester linkages, and can be DNA or RNA. In some aspects, the additional 5′ and/or 3′ end nucleosides are alternative nucleosides which can for example be included to enhance nuclease stability or for ease of synthesis.

In some aspects, the oligonucleotides utilize “altimer” design and comprise alternating 2′FANA and non-modified DNA regions that are alternated every three nucleosides. Altimer oligonucleotides are discussed in more detail in Min, et al., Bioorganic & Medicinal Chemistry Letters, 2002, 12(18): 2651-2654 and Kalota, et al., Nuc. Acid Res. 2006, 34(2): 451-61 (herein incorporated by reference).

In some aspects, the oligonucleotides utilize “hemimer” design and comprise a single 2′-modified flanking sequence adjacent to (on either side of the 5′ or the 3′ side of) a DNA core sequence. Hemimer oligonucleotides are discussed in more detail in Geary et al., 2001, J. Pharm. Exp. Therap., 296: 898-904 (herein incorporated by reference).

It will be understood that, although the sequence in SEQ ID NO: 1 is described as unmodified and/or un-conjugated sequence, the nucleosides of the oligonucleotide of SEQ ID NO:1 can comprise one or more alternative nucleosides and/or alternative linkages.

A skilled artisan is well aware that oligonucleotides having a structure of between about 18-20 base pairs can be particularly effective in inducing RNase H-mediated degradation. However, one can appreciate that shorter or longer oligonucleotides can be effective. In the aspects described above, by virtue of the nature of the oligonucleotide sequences provided herein, oligonucleotides described herein can include shorter or longer oligonucleotide sequences.

Without being bound by any theory, the oligonucleotides described herein can function via nuclease mediated degradation of the target nucleic acid, where the oligonucleotides are capable of recruiting a nuclease, such as an endonuclease like endoribonuclease (RNase) (e.g., RNase H). In some aspects, the oligonucleotides comprise a region of at least 5 or 6 DNA nucleosides that is flanked on one side or both sides by affinity enhancing alternative nucleosides, for example gapmers, headmers, and tailmers.

The RNase H activity of an oligonucleotide refers to its ability to recruit RNase H when in a duplex with a complementary RNA molecule. WO01/23613 provides in vitro methods for determining RNase H activity, which can be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using an oligonucleotide having the same base sequence as the modified oligonucleotide being tested, but containing only DNA monomers, with phosphorothioate linkages between all monomers in the oligonucleotide, and using the methodology provided by Example 91-95 of WO01/23613 (hereby incorporated by reference).

Furthermore, the oligonucleotides described herein identify a site(s) in a LINE-1 transcript that is susceptible to RNase H-mediated cleavage. In some aspects, an oligonucleotide is said to target within a particular site of an RNA transcript if the oligonucleotide promotes cleavage of the transcript anywhere within that particular site. In some aspects, an oligonucleotide will include at least about 21 contiguous linked nucleosides as described herein, coupled to additional linked nucleoside sequences taken from the region contiguous to the selected sequence in a LINE-1 gene.

In some aspects, oligonucleotides with homology sufficient to provide sequence specificity required to uniquely degrade an RNA can be designed using programs known in the art. In some aspects, systematic testing of several designed species for optimization of the inhibitory oligonucleotide sequence is undertaken in accordance with the teachings provided herein. Considerations when designing interfering oligonucleotides include, but are not limited to, biophysical, thermodynamic, and structural considerations, base preferences at specific positions, and homology. The making and use of inhibitory therapeutic agents based on non-coding oligonucleotides are also known in the art.

Further, it is contemplated that for any sequence identified herein, further optimization could be achieved by systematically either adding or removing linked nucleosides to generate longer or shorter sequences and testing those sequences generated by walking a window of the longer or shorter size up or down the target RNA from that point. Again, coupling this approach to generating new candidate targets with testing for effectiveness of oligonucleotides based on those target sequences in an inhibition assay as known in the art and/or as described herein can lead to further improvements in the efficiency of inhibition.

Further still, such optimized sequences can be adjusted by, e.g., the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

In some aspects, the oligonucleotide exhibits at least 50% mRNA inhibition at a 1 μM oligonucleotide concentration when determined using a cell assay when compared with a control cell. In some aspects, the oligonucleotide exhibits at least 60% mRNA inhibition at a 1 μM oligonucleotide concentration when determined using a human progeroid cell assay when compared with a control cell. In some aspects, the oligonucleotide exhibits at least 70% mRNA inhibition at a 1 μM oligonucleotide concentration when determined using a human progeroid cell assay when compared with a control cell. In some aspects, the oligonucleotide exhibits at least 80% mRNA inhibition at a 1 μM oligonucleotide concentration when determined using a human progeroid cell assay when compared with a control cell. In some aspects, the oligonucleotide exhibits at least 90% mRNA inhibition at a 1 μM oligonucleotide concentration when determined using a human progeroid cell assay when compared with a control cell. In some aspects, the oligonucleotide exhibits at least 95% mRNA inhibition at a 1 μM oligonucleotide concentration when determined using a human progeroid cell assay when compared with a control cell. In some aspects, the oligonucleotide exhibits at least 99% mRNA inhibition at a 1 μM oligonucleotide concentration when determined using a human progeroid cell assay when compared with a control cell.

In some aspects, the oligonucleotide exhibits at least 50% mRNA inhibition at a 20 nM oligonucleotide concentration when determined using a cell assay when compared with a control cell. In some aspects, the oligonucleotide exhibits at least 60% mRNA inhibition at a 20 nM oligonucleotide concentration when determined using a human progeroid cell assay when compared with a control cell. In some aspects, the oligonucleotide exhibits at least 70% mRNA inhibition at a 20 nM oligonucleotide concentration when determined using a human progeroid cell assay when compared with a control cell. In some aspects, the oligonucleotide exhibits at least 80% mRNA inhibition at a 20 nM oligonucleotide concentration when determined using a human progeroid cell assay when compared with a control cell. In some aspects, the oligonucleotide exhibits at least 90% mRNA inhibition at a 20 nM oligonucleotide concentration when determined using a human progeroid cell assay when compared with a control cell. In some aspects, the oligonucleotide exhibits at least 95% mRNA inhibition at a 20 nM oligonucleotide concentration when determined using a human progeroid cell assay when compared with a control cell. In some aspects, the oligonucleotide exhibits at least 99% mRNA inhibition at a 20 nM oligonucleotide concentration when determined using a human progeroid cell assay when compared with a control cell.

In some aspects, the oligonucleotide exhibits at least 50% mRNA inhibition at a 2 nM oligonucleotide concentration when determined using a cell assay when compared with a control cell. In some aspects, the oligonucleotide exhibits at least 60% mRNA inhibition at a 2 nM oligonucleotide concentration when determined using a human progeroid cell assay when compared with a control cell. In some aspects, the oligonucleotide exhibits at least 70% mRNA inhibition at a 2 nM oligonucleotide concentration when determined using a human progeroid cell assay when compared with a control cell. In some aspects, the oligonucleotide exhibits at least 80% mRNA inhibition at a 2 nM oligonucleotide concentration when determined using a human progeroid cell assay when compared with a control cell. In some aspects, the oligonucleotide exhibits at least 90% mRNA inhibition at a 2 nM oligonucleotide concentration when determined using a human progeroid cell assay when compared with a control cell. In some aspects, the oligonucleotide exhibits at least 95% mRNA inhibition at a 2 nM oligonucleotide concentration when determined using a human progeroid cell assay when compared with a control cell. In some aspects, the oligonucleotide exhibits at least 99% mRNA inhibition at a 2 nM oligonucleotide concentration when determined using a human progeroid cell assay when compared with a control cell.

In some aspects, the human progeroid cell assay uses HGPS^{-/-} cells or WRN^{-/-} cells.

In some aspects, the cell assay can comprise transfecting mammalian (e.g., human) cells, such as HGPS^{-/-} cells or WRN^{-/-} cells, HEK293, NIH3T3, or HeLa cells, with the desired concentration of oligonucleotide (e.g., 1 μM, 20 nM, or 2 nM) using Lipofectamine 2000 (Invitrogen) and comparing LINE-1 mRNA levels of transfected cells to LINE-1 levels of control cells. In some aspects, control cells are transfected with oligonucleotides not specific to LINE-1 or mock transfected. mRNA levels can be determined using RT-qPCR and LINE-1 mRNA levels can be normalized to GAPDH mRNA levels. The percent inhibition can be calculated as the percent of LINE-1 mRNA concentration relative to the LINE-1 concentration of the control cells.

In some aspects, oligonucleotides as disclosed herein are used in vectors for expression of the oligonucleotides in cells. Conventional techniques which do not require detailed explanation to one of ordinary skill in the art can be used to construct the vectors. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art. As used herein, the term “promoter” refers to DNA sequence capable of controlling the expression of a coding sequence or functional RNA. In some aspects, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

In the vectors, the oligonucleotide containing nucleic acid is operably linked to a promoter. The term “operably linked” refers to genetic elements that are joined together in a manner that enables them to carry out their normal functions. For example, a gene is operably linked to a promoter when its transcription is under the control of the promoter and this transcription results in the production of the product encoded by the gene.

In some aspects, an oligonucleotide is chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In some aspects, a ligand alters the distribution, targeting, or lifetime of an oligonucleotide into which it is incorporated. In some aspects, a ligand provides an enhanced affinity for a selected target, e.g., molecule, cell or cell type, compartment, e.g., a cellular or organ compartment, tissue, organ, or region of the body, as, e.g., compared to a species absent such a ligand.

In some aspects, ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some aspects, ligands include targeting groups, e.g., a cell or tissue targeting agent, e.g., a lectin, glycoprotein, lipid or protein, e.g., an antibody, that bind to a specified cell type such as a kidney cell. In some aspects, a targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some aspects, a ligand is a protein, e.g., glycoprotein, or peptide, e.g., a molecule having a specific affinity for a co-ligand, or antibodies e.g., an antibody, that binds to a specified cell type such as a hepatic cell. Ligands can include hormones and hormone receptors. They can include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some aspects, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable as ligands (e.g. as PK modulating ligands). In addition, aptamers that bind serum components (e.g. serum proteins) are also suitable for use as PK modulating ligands in the aspects described herein.

Ligand-conjugated oligonucleotides can be synthesized by the use of an oligonucleotide that bears a pendant reactive functionality, such as that derived from the attachment of a linking molecule onto the oligonucleotide (described below). This reactive oligonucleotide can be reacted directly with commercially-available ligands, ligands that are synthesized bearing any of a variety of protecting groups, or ligands that have a linking moiety attached thereto.

The oligonucleotides used in the conjugates can be conveniently and routinely made through the well-known technique of solid-phase synthesis. Equipment for such synthesis is sold by several vendors including, for example, Applied Biosystems (Foster City, Calif.). Any other means for such synthesis known in the art can additionally or alternatively be employed. It is also known to use similar techniques to prepare other oligonucleotides, such as the phosphorothioates and alkylated derivatives.

In the ligand-conjugated oligonucleotides, such as the ligand-molecule bearing sequence-specific linked nucleosides, the oligonucleotides and oligonucleosides can be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using conjugate precursors that already bear a linking moiety, the synthesis of the sequence-specific linked nucleosides is typically completed, and the ligand molecule is then reacted with the linking moiety to form the ligand-conjugated oligonucleotide. In some aspects, the oligonucleotides or linked nucleosides are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In some aspects, the ligand or conjugate is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. A lipid or lipid-based ligand can (a) increase resistance to degradation of the conjugate, (b) increase targeting or transport into a target cell or cell membrane, and/or (c) be used to adjust binding to a serum protein, e.g., HSA.

In an some aspects, the ligand is a moiety, e.g., a vitamin, which is taken up by a target cell, e.g., a proliferating cell. Exemplary vitamins include vitamin A, E, and K.

ii. Cell Permeation Agents

In some aspect, the ligand is a cell-permeation agent, such as a helical cell-permeation agent. In one aspect, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In one aspect, the helical agent is an alpha-helical agent, which can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ and the Drosophila Antennapedia protein (RQIKIWFQNRRMKWKK have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods can be linear or cyclic, and can be modified, e.g., glycosylated or methylated, to facilitate targeting to a specific tissue(s). RGD-containing peptides and peptidiomimemtics can include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some aspects, an oligonucleotide further comprises a carbohydrate. The carbohydrate conjugated oligonucleotides are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In some aspects, a carbohydrate conjugate for use in the compositions and methods described herein is a monosaccharide. In some aspects, the carbohydrate conjugate further comprises one or more additional ligands as described above, such as, but not limited to, a PK modulator and/or a cell permeation peptide. Additional carbohydrate conjugates (and linkers) suitable for use include those described in PCT Publication Nos. WO 2014/179620 and WO 2014/179627, the entire contents of each of which are incorporated herein by reference.

iv. Linkers

In some aspects, the conjugate or ligand described herein can be attached to an oligonucleotide with various linkers that can be cleavable or non-cleavable.

Linkers typically comprise a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one aspect, the linker is between about 1-24, 2-24, 3-24, 4-24, 5-24, 6-24, 6-18, 7-18, 8-18, 7-17, 8-17, 6-16, 7-17, 8-16 or 1, 2, 3, 4, 5, 6, 7, 8, 9, 10, 11, 12, 13, 14, 15, 16, 17, 18, 19, 20, 21, 21, 22, 23, or 24 atoms.

A cleavable linking group is one which is sufficiently stable outside the cell, but which upon entry into a target cell is cleaved to release the two parts the linker is holding together. In some aspects, the cleavable linking group is cleaved at least about 10 times, 20 times, 30 times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to test the candidate cleavable linking group for the ability to resist cleavage in the blood or when in contact with other non-target tissue. Thus, one can determine the relative susceptibility to cleavage between at least two conditions, where at least one condition is selected to be indicative of cleavage in a target cell and another condition is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In some aspects, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

a. Redox Cleavable Linking Groups

In one aspect, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can be evaluated under conditions which are selected to mimic blood or serum conditions. In one aspect, candidate compounds are cleaved by at most about 10% in the blood. In other aspects, useful candidate compounds are degraded at least about 2, 4, 10, 20, 30, 40, 50, 60, 70, 80, 90, or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another aspect, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—,
—O—P(S)(OR^{k})—O—, —O—P(S)(SR)—O—, —S—P(O)(OR^{k})—O—, —O—P(O)(OR)—S—, —S—P(O)(OR^{k})—S—,
—O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—,
—S—P(O)(R^{k})—S—, —O—P(S)(R^{k})—S—. These candidates can be evaluated using methods analogous to those described above.
   c. Acid Cleavable Linking Groups

In another aspect, a cleavable linker comprises an acid cleavable linking group. An acid cleavable linking group is a linking group that is cleaved under acidic conditions. In some aspects, acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). In one aspect, the carbon is attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another aspect, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another aspect, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids. These candidates can be evaluated using methods analogous to those described above.

In some aspects, an oligonucleotide is conjugated to a carbohydrate through a linker. Linkers include bivalent and trivalent branched linker groups. Linkers for oligonucleotide carbohydrate conjugates include, but are not limited to, those described in formulas 24-35 of PCT Publication No. WO 2018/195165.

Representative U.S. patents that teach the preparation of oligonucleotide conjugates include, but are not limited to, U.S. Pat. Nos. 6,294,664; 6,320,017; 6,576,752; 6,783,931; 6,900,297; 7,037,646; 8,106,022, each of which are hereby incorporated herein by reference.

It is not necessary for all positions in a given compound to be uniformly modified, and in fact more than one of the aforementioned modifications can be incorporated in a single compound or even at a single nucleoside within an oligonucleotide. Oligonucleotide compounds that are chimeric compounds are also contemplated. Chimeric oligonucleotides typically contain at least one region wherein the RNA is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxy oligonucleotides hybridizing to the same target region. Cleavage of the RNA target can be routinely detected by gel electrophoresis and, if necessary, associated nucleic acid hybridization techniques known in the art.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### III. Formulations and Modes of Administration

In certain embodiments, the present disclosure provides formulations or pharmaceutical compositions suitable for the therapeutic delivery of antisense oligonucleotides, as described herein. Hence, in certain embodiments, the present disclosure provides pharmaceutically acceptable compositions that comprise a therapeutically-effective amount of one or more of the oligonucleotides described herein, formulated together with one or more pharmaceutically acceptable carriers (additives) and/or diluents. While it is possible for an oligonucleotide of the present disclosure to be administered alone, it is preferable to administer the compound as a pharmaceutical formulation (composition).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including the oligomers of the present disclosure.

As detailed below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligomers of the instant disclosure include: PEG conjugated nucleic acids, phospholipid conjugated nucleic acids, nucleic acids containing lipophilic moieties, phosphorothioates, β-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

In some aspects, the composition comprises surface-modified liposomes containing poly (ethylene glycol) lipids (PEG-modified, branched and unbranched or combinations thereof, or long-circulating liposomes or stealth liposomes). Oligonucleotides of the disclosure can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tissues, presumably by extravasation and capture in the target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In some aspects, provided are oligonucleotide pharmaceutical compositions prepared for delivery. In some aspects, oligonucleotides of the present disclosure are in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, the present disclosure provides antisense oligomers in pharmaceutical compositions comprising gluconic-acid-modified polyhistidine or gluconylated-polyhistidine/transferrin-polylysine. One skilled in the art will also recognize that amino acids with properties similar to His and Lys may be substituted within the composition.

In some aspects, the compositions further comprise wetting agents, emulsifiers and lubricants, such as sodium lauryl sulfate and magnesium stearate, as well as coloring agents, release agents, coating agents, sweetening, flavoring and perfuming agents, preservatives and antioxidants.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some aspects, the formulations of the disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some aspects, a formulation of the disclosure comprises an excipient selected from cyclodextrins, celluloses, liposomes, micelle forming agents, e.g., bile acids, and polymeric carriers, e.g., polyesters and polyanhydrides; and an oligonucleotide described herein. In some aspects, a formulation renders orally bioavailable an oligonucleotide described herein.

Methods of preparing these formulations or pharmaceutical compositions include a step of bringing into association an oligonucleotide of the present disclosure with the carrier and, optionally, one or more accessory ingredients. In general, the formulations are prepared by uniformly and intimately bringing into association a oligonucleotide of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. An oligonucleotide as described herein may also be administered as a bolus, electuary or paste.

In solid dosage forms of the disclosure for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid pharmaceutical compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid pharmaceutical compositions which can be dissolved in sterile water, or some other sterile injectable medium immediately before use. These pharmaceutical compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral pharmaceutical compositions can also include adjuvants such as wetting agents, emulsifying and suspending agents, sweetening, flavoring, coloring, perfuming and preservative agents.

Suspensions, in addition to the active oligonucleotide, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more oligonucleotides described herein with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active oligonucleotides may be mixed under sterile conditions with a pharmaceutically-acceptable carrier, and with any preservatives, buffers, or propellants which may be required. The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligonucleotide described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligonucleotide described herein to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more oligonucleotides of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the oligonucleotides in biodegradable polymers such as polylactide-polyglycolide. Depending on the ratio of oligonucleotide to polymer, and the nature of the particular polymer employed, the rate of oligomer release can be controlled. Examples of other biodegradable polymers include poly(orthoesters) and poly(anhydrides). Depot injectable formulations may also prepared by entrapping the drug in liposomes or microemulsions that are compatible with body tissues.

When the oligonucleotides described herein are administered as pharmaceuticals to humans, they can be given per se or as a pharmaceutical composition containing, for example, 0.1 to 99% (more preferably, 10 to 30%) of active ingredient in combination with a pharmaceutically acceptable carrier.

As noted above, the formulations or preparations of the present disclosure may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the oligonucleotides described herein, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligonucleotide employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the compounds of the disclosure employed in the pharmaceutical composition at levels lower than that required in order to achieve the desired therapeutic effect and gradually increase the dosage until the desired effect is achieved. In general, a suitable daily dose of a compound of the disclosure will be that amount of the compound which is the lowest dose effective to produce a therapeutic effect. Such an effective dose will generally depend upon the factors described above. Generally, oral, intravenous, intracerebroventricular and subcutaneous doses of the compounds of this disclosure for a patient, when used for the indicated effects, will range from about 0.0001 to about 100 mg per kilogram of body weight per day.

In some embodiments, the oligonucleotides described herein are administered in doses generally from about 1-10 mg/kg. In some aspects, doses of greater than 10 mg/kg may be necessary. In some aspects, doses for i.v. administration are from about 0.5 mg to 10 mg/kg.

In some aspects, dosing is one administration per day. In certain aspects, dosing is one or more administration every 1 or 4 months, as needed, to maintain the desired therapeutic effect.

Oligonucleotides can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In some aspects, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In some aspects, the formulations contain micelles formed from an oligonucleotide described herein and at least one amphiphilic carrier, in which the micelles have an average diameter of less than about 100 nm. In some aspects, micelles having an average diameter less than about 50 nm are used with oligonucleotides described herein. In some aspects, micelles having an average diameter less than about 30 nm, or even less than about 20 nm are used.

While all suitable amphiphilic carriers are contemplated, the presently preferred carriers are generally those that have Generally-Recognized-as-Safe (GRAS) status, and that can both solubilize the oligondescribed herein and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In some aspects, the delivery may occur by use of liposomes, nanocapsules, microparticles, microspheres, lipid particles, vesicles, and the like, for the introduction of the pharmaceutical compositions into suitable host cells. In particular, the pharmaceutical compositions may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use with oligonucleotides described herein are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In some aspects, polymers have a molecular weight of from about 100 or 120 daltons up to about 5,000 or 10,000 daltons, or from about 300 daltons to about 5,000 daltons. In some aspects, the polymer is polyethyleneglycol having a molecular weight of from about 100 to about 5,000 daltons, or having a molecular weight of from about 300 to about 5,000 daltons. In some aspects, the polymer is polyethyleneglycol of 750 daltons (PEG(750)). Polymers may also be defined by the number of monomers therein; in some aspects, polymers of at least about three monomers, such PEG polymers consisting of three monomers (approximately 150 daltons) are used.

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In some aspects, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

In some aspects, formulations comprising liposomes containing an oligonucleotide are used where the liposome membrane is formulated to provide a liposome with increased carrying capacity. Alternatively or in addition, the compound of the present disclosure may be contained within, or adsorbed onto, the liposome bilayer of the liposome. An oligonucleotide described herein may be aggregated with a lipid surfactant and carried within the liposome's internal space; in these cases, the liposome membrane is formulated to resist the disruptive effects of the active agent-surfactant aggregate.

In some aspects, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes disclosed herein are in solubilized form. Aggregates of surfactant and active agent (such as emulsions or micelles containing the active agent of interest) may be entrapped within the interior space of liposomes according to the present disclosure. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present disclosure.

Liposomes according to the present disclosure may be prepared by any of a variety of techniques that are known in the art. See, e.g., U.S. Pat. No. 4,235,871; Published PCT applications WO 96/14057; New RRC, Liposomes. A practical approach, IRL Press, Oxford (1990), pages 33-104; Lasic DD, Liposomes from physics to applications, Elsevier Science Publishers BV, Amsterdam, 1993. For example, liposomes described herein may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In some aspects of the formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In some aspects, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In some aspects, reagents such as DharmaFECT® and Lipofectamine® may be utilized to introduce polynucleotides or proteins into cells.

The release characteristics of a formulation of the present disclosure depend on the encapsulating material, the concentration of encapsulated oligonucleotides, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An oligonucleotide may be formulated to be contained within, or, adapted to release by a surgical or medical device or implant. In some aspects, an implant may be coated or otherwise treated with an oligonucleotide. For example, hydrogels, or other polymers, such as biocompatible and/or biodegradable polymers, may be used to coat an implant with the pharmaceutical compositions of the present disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the oligonucleotides for use according to the disclosure may be formulated for administration in any convenient way for use in human medicine, by analogy with other pharmaceuticals. The antisense oligonucleotides and their corresponding formulations may be administered alone or in combination with other therapeutic strategies in the treatment of progeria syndrome, premature aging, signs and/or symptoms of aging such as stem cell therapies, administration of reverse transcriptase inhibitors, and therapies that counter the aging process in cells.

In some aspects, the additional therapeutic may be administered prior, concurrently, or subsequently to the administration of the oligonucleotides described herein. For example, the oligonucleotides may be administered in combination with an additional therapeutic. In some aspects, the oligonucleotides are administered to a patient that is on a background anti-aging therapy. For example, in some aspects, the patient has been treated with an antiaging therapy prior to administration of an antisense oligonucleotide and continues to receive the antiaging therapy.

### IVI. Methods of Use

Provided are methods of treating, preventing, or inhibiting premature aging or an age-related disease in a subject, the method comprising administering a therapeutically effective amount of an oligonucleotide described herein, a pharmaceutical composition described herein, or a composition described herein to the subject in an amount effective to treat, prevent, or inhibit premature aging or an age-related disease in the subject.

In some aspects, the subject has a progeroid syndrome.

In some aspects, the progeroid syndrome is selected from the group consisting of Hutchinson-Gilford progeria syndrome; Werner syndrome; atypical progeria, mandibuloacral dysplasia type A; mandibuloacral dysplasia type B; mandibuloacral dysplasia associated to MTX2; mandibular hypoplasia, progeroid features and lipodystrophy syndrome (MDPL); Nestor-Guillermo progeria syndrome; and restrictive dermopathy.

In some aspects, the subject has a mutation in a LMNA gene, ZMPSTE24 gene, BANF1 gene, POLD1 gene, MTX2 gene, or WRN gene.

In some aspects, the subject has signs and/or symptoms of aging.

In some aspects, methods of treating, preventing or inhibiting signs or symptoms of aging in a subject are provided, the methods comprising administering a therapeutically effective amount of an oligonucleotide described herein, a pharmaceutical composition described herein, or a composition described herein to the subject in an amount effective to treat, prevent, or inhibit signs and symptoms of aging in the subject.

In some aspects, methods of decreasing LINE-1 RNA in a subject are provided, the methods comprising administering a therapeutically effective amount of an oligonucleotide described herein, a pharmaceutical composition described herein, or a composition described herein to the subject in an amount effective to decrease LINE-1 RNA in the subject.

In some aspects, methods of rejuvenating a tissue in a subject experiencing pre-mature aging or having an age-related disease are provided, the methods comprising administering a therapeutically effective amount of an oligonucleotide described herein, a pharmaceutical composition described herein, or a composition described herein to the subject in an amount effective to rejuvenate a tissue in the subject.

In some aspects, methods of reversing signs or symptoms of aging in a tissue of a subject are provided, the methods comprising administering a therapeutically effective amount of an oligonucleotide described herein, a pharmaceutical composition described herein, or a composition described herein to the subject in an amount effective to reverse signs or symptoms of aging in a tissue of the subject.

In some aspects, the methods further comprise administering an additional therapeutic agent to the subject. In some aspects, the additional therapeutic agent is an aging-related therapeutic agent. In some aspects, an oligonucleotide described herein, a pharmaceutical composition described herein, or a composition described herein are administered by an intravenous, intramuscular, intradermal, subcutaneous, intraperitoneal, or intranasal route.

The process of administration can be varied, depending on the composition, or compositions, and the desired effect. Thus, the process of administration involves administering a therapeutic agent (e.g., any composition or pharmaceutical composition, oligonucleotide, polynucleotide, or vector disclosed herein) to a patient in need of such treatment.

Administration can be accomplished by any means appropriate for the therapeutic agent, for example, by parenteral, mucosal, pulmonary, subcutaneous, intradermal, topical, catheter-based, or oral means of delivery. Parenteral delivery can include for example, subcutaneous, intravenous, intramuscular, intra-arterial, intraperitoneal, intralymphatic, and injection into the tissue of an organ. Mucosal delivery can include, for example, intranasal delivery, administration into an airway of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods and compositions described herein.

In some aspects, a therapeutically effective amount of an oligonucleotide, a pharmaceutical composition, or a composition is administered by an intravenous, intramuscular, intradermal, subcutaneous, intraperitoneal, or intranasal route.

In some aspects, an oligonucleotide or pharmaceutical composition or composition is delivered to a subject or a tissue of a subject as a combination therapy.

In some aspects, an oligonucleotide or pharmaceutical composition or composition is delivered to a subject or a tissue of a subject together with at least one additional therapeutic agent. In some aspects, the additional therapeutic agent is an aging-related therapeutic agent. In some aspects, the oligonucleotide, pharmaceutical composition or composition is administered to a subject before, after, or together with at least one additional therapeutic agent.

In some aspects, the oligonucleotide, pharmaceutical composition, or composition and the at least one additional therapeutic agent can be administered by the same route or by different routes; at essentially the same time (i.e. simultaneously, concurrently) or at different times (e.g. sequentially, successively, alternately, consecutively, or according to any other sort of alternating regime).

For example, a first therapeutic composition of the combination may be administered by intravenous injection while the additional therapeutic composition of the combination may be administered intradermally. Alternatively, for example, all therapeutic compositions may be administered by intravenous injection or all therapeutic compositions may be administered by intradermal injection.

### V. Kits

Kits including (a) a pharmaceutical composition including an oligonucleotide agent that reduces the level and/or activity of LINE-1 in a cell or subject described herein, and (b) a package insert with instructions to perform any of the methods described herein are contemplated. In some aspects, the kit includes (a) a pharmaceutical composition including an oligonucleotide agent that reduces the level and/or activity of LINE-1 in a cell or subject described herein, (b) an additional therapeutic agent, and (c) a package insert or label with instructions, e.g., comprising administration schedules, to allow a practitioner (e.g., a physician, nurse, or patient) to administer the composition contained therein to a patient or perform any of the methods described herein. The kit also can include device for administering the pharmaceutical composition, e.g., a syringe.

The details of one or more aspects are set forth in the description below. Other features, objects, and advantages will be apparent from the description and from the claims.

### EXAMPLES

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

The breadth and scope of the present invention should not be limited by any of the above-described exemplary embodiments, but should be defined only in accordance with the following claims and their equivalents.

### Example 1—Materials and Methods

### Study Design

This disclosure reports the investigation of the role of L1 RNAs in accelerated aging syndromes and demonstrates that L1 RNA depletion can prevent or delay the onset of the aged and senescent phenotypes. First, we tested the effects of L1 ASO ex-vivo in primary dermal fibroblasts from patients with HGPS, WRN and APS, as well as mesenchymal stem cells from patients with HGPS and WRN. We investigated the expression profile of SASP genes and the dynamics of heterochromatin-associated histone marks. Further, we assessed whether L1 RNA accumulation was a cause or a consequence of senescence ex-vivo using TET-inducible GFP-progerin in human dermal fibroblasts. All animal procedures were performed according to NIH guidelines and approved by the Committee on Animal Care at the Salk Institute. Using the LAKI progeria mouse model, we tested L1 ASO effects on delaying tissue degeneration and increasing the lifespan of the animals. We performed RNA-seq to study the transcriptional profile of the mice tissues upon L1 ASO treatment. Male and female mice were used in the study and animals were randomly assigned between the experimental groups. Sample sizes were not predetermined, and more than 10 animals were used for the in-vivo study. All the experiments were independently repeated at least three times. Figure legends contain sample size and replicate information.

### Animals and In Vivo Treatments

All animal procedures were performed according to NIH guidelines and approved by the Committee on Animal Care at the Salk Institute and Universidad Católica San Antonio de Murcia (UCAM). The mouse model of Hutchinson-Gilford progeria syndrome (HGPS) carrying the LMNA mutation G609G (LAKI) was generated by C. López-Otin at the University of Oviedo, Spain and kindly donated by B. Kennedy at the Buck Institute. Experiments with WT and LAKI homozygous mice were performed with both genders. For lifespan experiments, mice of both genders from a litter were randomly assigned to control and experimental groups. Any animals that appeared unhealthy before the start of experiments were excluded. No inclusion criterion was used. The mice were housed with a 12 hr light/dark cycle between 06:00 and 18:00 in a temperature-controlled room (22±1C) with free access to water and food. The colony is maintained without floor feeding. L1-specific or scramble 2′-deoxy-2′fluoro-β-d-arabino-nucleotides (FANA ASOs) were delivered three times by intraperitoneal injection at the dose of 2 mg/kg with a 10 day interval starting at 8 weeks of age. ASOs were designed on the L1-ORF1 of the L1 consensus sequence to target full-length L1 transcripts belonging to the L1-Ta family in humans and L1-Tf, -Gf and -A families in the mouse. The ASOs with NCBI BLAST E-score higher than 0.1, corresponding to non-significant homology, were selected to exclude off-target effects on known genes (both coding and non-coding). ASOs used in these experiments are shown in Table 3 below.

### Cell Culture

Tail tip fibroblasts (TTFs) were isolated from WT and LAKI mice and cultured at 37° C. in Dulbecco's Modified Eagle Medium (DMEM) (Invitrogen) containing Gluta-MAX, non-essential amino acids, and 10% fetal bovine serum (FBS, Gibco). WT and HGPS human dermal fibroblast were obtained from Coriell Biobank (WT: AG06234, AG10108, AG09309, AG10803, AG03257. HGPS: AG06917, AG11498, AG10578, AG11572, AG06297. APS: AG041100 (LMNA E578V), AG00989 (LMNA R644C), AG00990 (LMNA T10I), AG09233 (unknown mutation). WRN: AG12798; AG24467; AG06300; AG00780; AG05229). For LINE-1 knockdown, cells were incubated with 1 μM FANA ASO dissolved in culture medium (61) every 4 days and collected after two passages for senescent marker expression or immunohistochemistry. 3TC (Sigma-Aldrich) treatments were performed at 10 mM as in De Cecco et al. (11). Translation inhibitor ASOs (miRCURY LNA, Qiagen) were used at a final concentration of 20 nM and transfected with RNAiMAX (Invitrogen, Thermo Fisher Scientific) following manufacturer instructions. For L1 overexpression, WT TTFs were transfected with pTNC7 plasmid containing a full-length L1spa element under the control of the endogenous 5′UTR promoter region (61, 62), kindly donated by E. Heard. For p16 and p21 overexpression, WT TTFs were transfected with commercial expression vectors containing p16-HA (EX-Mm01718-Lv186, Genecopoeia) and p21-FLAG (EX-Mm01715-Lvl58, Genecopoeia). Cells were transfected with Lipofectamine3000 (Invitrogen) and collected 72 hours post-transfection. SUV39H1 overexpression was performed by infecting cells with an adenoviral vector containing SUV39H1 cassette (Applied Biological Materials, #459110510200) at a dosage of 5 multiplicity of infection (MOI). An empty adenovirus was used as control. SUV39H1 knockdown was performed by infecting cells with a lentivirus expressing a set of four pooled SUV39H1-specific siRNA (Applied Biological Materials, #459110910296) at a dose of 5 MOI. An empty lentiviral vector was used as a control. Human MSCs were differentiated from patient-derived pluripotent stem cells and cultured as previously described in Zhang et al. (8). Briefly, MSCs were maintained in DMEM 1 g/l glucose medium (Invitrogen) with 10% FBS, 1% penicillin/streptomycin (Gibco), 10 ng/ml bFGF (JPC).

### Chromatin Immunoprecipitation

ChIP was performed as in Hosny & Della Valle et al. and Liu et al. (63-65). Briefly, cells were cross-linked in 1% formaldehyde (Thermo Fisher Scientific, 28906) for 10 min at room temperature. Cross-linked cells were lysed in lysis buffer 1 (50 mM HEPES KOH, pH 7.5, 10 mM NaCl, 1 mM EDTA, 10% glycerol, 0.5% NP-40, 0.25% Triton X-100) overnight. Nuclei were collected, washed in lysis buffer 2 (10 mM Tris-HCl, pH 8, 200 mM NaCl, 1 mM EDTA, 0.5 mM EGTA) and lysed in lysis buffer 3 (10 mM Tris-HCl, pH 8, 100 mM NaCl, 1 mM EDTA, 0.5 mM EGTA, 0.1% Na-deoxycholate, 0.5% N-lauroylsarcosine). Freshly prepared 1×protease inhibitor cocktail was added into all lysis buffers. Chromatin was sheared (BRANSON A250 with a 3.2-mm tapered microtip; four to five cycles of 2 min at 20% amplitude, 50% of duty cycle). In each IP reaction, 100 μg of chromatin DNA equivalents (DNA concentration measured with Nanodrop) were incubated overnight with 3 g of antibody. The immunocomplexes were recovered with magnetic Dynabeads (Protein A; Invitrogen) for 2 h and washed on the wheel at 4° C. for 5 min with low-salt (LS) wash buffer (0.1% SDS, 2 mM EDTA, 1% Triton X-100, 20 mM Tris-HCl, pH 8, 150 mM NaCl) and high-salt (HS) wash buffer (0.1% SDS, 2 mM EDTA, 1% Triton X-100, 20 mM Tris-HCl, pH 8, 500 mM NaCl). Then, LS and HS buffer washes were repeated one more time. The final wash was carried out with Tris 10 mM-EDTA 1 mM (TE) buffer plus 150 mM NaCl twice. Precipitated DNA was eluted using elution buffer (50 mM Tris-HCl, pH 8, 10 mM EDTA, 1% SDS) at 65° C. for 15 min. For de-cross-linking, all eluted samples were incubated at 65° C. overnight. Chromatin was digested with RNase A (0.2 mg/ml) and proteinase K (0.2 mg/ml), and DNA was purified for qPCR analysis with phenol-chloroform extraction. H3K9me3 ChIP results are expressed as percentage of input. The qPCR primers used for ChIP analysis are listed in Table 1.

### Statistical Analysis

In bar plots, values are presented as means and Standard Error Mean (SEM.), and values for each replicate are shown. To determine the significance between two mean values, we made comparisons by two-tailed t test with Welch's correction. For multiple comparisons, Holm-Sidak correction method was used. Comparisons among three or more samples were made by one-way analysis of variance (ANOVA) applying the Brown-Forsythe correction. For all statistical tests, P<0.05 was accepted for significance. For the lifespan studies, both Mantel-Cox and Gehan-Breslow tests have been applied. All the plots and the statistical analyses were performed with GraphPad Prism8 software.

### Histological Analysis

For histological analysis, tissue samples were collected at 16 weeks of age after 8 weeks of ASO injection. Mice were perfused with PBS and 10% buffered formalin solution. Subsequently, tissues were fixed overnight at 4° C. in 10% buffered formalin solution and embedded in paraffin. 5-7 μm sections were used for hematoxylin and eosin staining (H&E) or immunohistochemistry.

### Immunohistochemistry

Cells were fixed with 4% formaldehyde in PBS at room temperature (RT) for 10 min. After fixation, cells were treated with 0.5% Triton X-100 in PBS for 5 min at RT. After blocking with 4% BSA in PBS for 30 min, cells were incubated at 4° C. overnight with the primary antibody, followed by washing in PBS and incubation at RT for 1 hr with the corresponding secondary antibody. Cells were mounted using DAPI-Fluoromount-G (SouthernBiotech). Confocal image acquisition was performed using a Zeiss LSM 780 laser-scanning microscope (Carl Zeiss Jena). Images were acquired as z-stacks with 0.20 μm intervals between sections using the adequate lasers (488-nm, 568-nm, 633-nm, and 405-nm). Z-stack 3D reconstruction, nuclei segmentation, and fluorescence signal quantification were performed using Imaris (v.8.4.1) using nuclei volume as a reference to normalize fluorescence signals. The laser intensity was typically set to 3%-5% transmission of the maximum intensity, and the settings were established to avoid signal saturation for any of the lasers. Tissues sections underwent permeabilization and antigen retrieval using HistoVT One (Nacalai Tesque). Subsequently, tissue sections were blocked with 5% fraction V BSA in PBS(Sigma-Aldrich) and immunoglobulin masking reagent (Vector Laboratories) and incubated overnight with primary antibody. Finally, tissue sections were incubated with secondary antibody in blocking buffer at room temperature for 60 min (Invitrogen). Tissue sections were mounted with DAPI Fluoromount-G mounting medium (Southern Biotech.). The list of the antibodies used is in Table 1.

TABLE 1

| Target | Antibody | Source | Antibody ID |
|---|---|---|---|
| SUV39H1 | Rabbit mAb #8729 | Cell Signaling | RRID:AB_{—} |
| | | Technology | 10829612 |
| SUV39H2 | Rabbit mAb | Abcam | RRID:AB_{—} |
| | ab190870 | | 2827544 |
| H3K9me3 | Rabbit pAb ab8898 | Abcam | RRID:AB_306848 |
| H3K27me3 | Mouse mAb ab6002 | Abcam | RRID:AB_305237 |
| H4K20me3 | Rabbit pAb ab9053 | Abcam | RRID:AB_306969 |
| Ki67-AF488 | Rabbit mAb | Abcam | |
| | ab197234 | | |
| pH2AX | Rabbit mAb #2577S | Cell Signaling | RRID:AB_2118010 |
| | | Technology | |
| pH2AX | Mouse mAb # | MERK- | |
| | 05-636 | Millipore | |
| 53BP1 | rabbit mAb | abcam | RRID:AB_2890610 |
| | ab175933 | | |
| Progerin | Mouse mAb | Sigma Aldrich | RRID:AB_{—} |
| | SAB4200272 | | 10743641 |
| total H3 | rabbit mAb | abcam | RRID:AB_2493104 |
| | ab176842 | | |
| Lamin B | Goat pAb SC-6216 | Santa Cruz | RRID:AB_648156 |
| | | Biotech | |
| HDAC2 | Rabbit mAb #57156 | Cell Signaling | RRID:AB_2756828 |
| | | Technology | |

### Fluorescent In Situ Hybridization

RNA-FISH or immuno-RNA FISH in TTFs and Tissue sections was performed according to the manufacturer's standard protocol (Biosearch Technologies) and as also previously reported (14). Fixation was performed in 3% paraformaldehyde (PFA) for 15 min, followed by permeabilization with 1% Triton X-100 for 5 minutes at room temperature prior to hybridization. Hybridization was performed at 38° C. overnight, using 48 single-molecule probes designed to span the length of the active mouse L1spa element recognizing the majority of transcribed LINE-1 RNAs. The probe set was designed and produced by Biosearch Technologies. Custom Stellaris® FISH Probes labeled with CalFluor610 were designed against L1spa by utilizing the Stellaris® FISH Probe Designer (Biosearch Technologies, Inc., Petaluma, CA) available online at www.biosearchtech.com/stellarisdesigner.

### RNA Immunoprecipitation

5×106 TTFs from both WT and LAKI mice were crosslinked through UV light irradiation (150 mJ/cm2 at 254 nm in a Stratalinker 2400). Cells were scraped and pelleted at 1500 rpm for 10 minutes at 4° C. Cells were incubated in RIPA buffer supplemented with 1× protease inhibitor complex (P.L.C., Roche), 0.5 mM D.T.T. (Sigma Aldrich), and 1× ProtectRNA RNase inhibitor (Sigma Aldrich) at 4° C. rocking on a wheel for 10 minutes. The lysate was diluted 1:1 with NLB lysis buffer (150 mM KCL, 25 mM Tris pH 7.5, 5 mM EDTA, 0.5% NP-40, 0.5 mM DTT, P.I.C. and ProtectRNA). The lysate was immunoprecipitated with 6 micrograms of antibody for 2 hours, rocking on a wheel at 4° C. The IP was recovered with 50 ul Protein G magnetic beads (Invitrogen) at 4° C., rocking on a wheel for 2 hours. Beads were washed 2 times in 1 ml of NLB buffer on a wheel for 10 minutes at 4° C. RNA was eluted from beads with proteinase K digestion for 30 minutes at 37° C. and purified as previously described with RNeasy mini kit plus (Qiagen). The list of the antibodies used is in Table 1.

LINE-1 RNA in vitro transcription and SUV39 enzymatic activity assay

LINE-1 RNA was in vitro transcribed using MAXIscript Transcription Kit (Invitrogen) using a plasmid containing the active L1 element as a template. The pBlueScript SKII vector containing the mouse active L1spa sequence was kindly donated by prof. Edith Heard. Before reaction, pDNA has been linearized with NotI restriction enzyme to transcribe the full-length sense LINE-1 RNA or XhoI restriction enzyme for antisense LINE-1 RNA. Transcribed RNA was purified with RNeasy mini kit (Qiagen) following the RNA clean-up protocol. Recombinant Suv39H1 (Activemotif) Histone methyltransferase (HMT) activity was assayed using EpiQuik™ Histone Methyltransferase Activity/Inhibition Assay Kit (Epigentek) following manufacturer instructions. Briefly, 1 g of recombinant SUV39H1 was incubated with 10 ng or 50 ng of in vitro transcribed sense LINE-1 RNA. Antisense LINE-1 RNA was used as a negative control as previously described in ref.(28). 1 g of SUV39H1 alone or complexed with RNA were used for the assay in parallel with 1 μl of positive control enzyme. Absorbance was read at 450 nm on a microplate reader and HMT activity was calculated as: HMT activity=OD (sample−blank)/incubation time (Hr).

### Chromatin Fractionation

The nuclei were isolated from the cytosolic fraction using ice cold 0.1% NP-40 in PBS. Nuclei and chromatin fractions were extracted using a gradient of salt concentration (33, 32). Briefly, nuclei pellet was resuspended in strip buffer (10 mM Tris-HCL Ph 7.4, 1 mM EGTA, 1.5 mM KCl, 5 mM MgCl2, 290 mM Sucrose, 0.1% Triton X-100, 1 mM DTT) for nucleosol extraction and medium salt buffer (20 mM HEPES, pH 7.9, 25% glycerol, 500 mM KCl, 1.5 mM MgCl2, 0.2 mM EDTA, 1 mM DTT, 1× Complete mini EDTA-free (Roche), and a high salt concentration buffer (1.5M mM KCl) to sequentially isolate different chromatin fractions. The remaining insoluble fraction was resuspended in 2× Laemmli buffer.

### DNA Methylation Studies and Epigenetic Clocks

DNA was extracted from cells using the Zymo Quick DNA mini-prep plus kit (D4069) according to the manufacturer's instructions and DNA methylation levels were measured on Illumina Infinium EPIC arrays which measures bisulfite-conversion-based, single-CpG resolution DNAm levels at 866 thousand CpG sites in the human genome. These data were generated by following the standard protocol of Illumina methylation assays, which quantifies methylation levels by the beta value using the ratio of intensities between methylated and un-methylated alleles. Thus, R values range from 0 (completely un-methylated) to 1 (completely methylated). We used the “noob” normalization method, which is implemented in the “minfi” Rpackage (65). In our primary clock analysis, we used the skin & blood clock (based on 391 CpGs) because it is tailor made for fibroblasts and it revealed epigenetic age acceleration effects in fibroblasts from Hutchinson Gilford progeria cases (37). In our secondary analysis we used the PhenoAge Clock because it predicts morbidity risk (36). The epigenetic clock estimates were calculated with the advanced option of the online epigenetic clock calculator (https://dnamage.genetics.ucla.edu/new) (38).

### Western Blot

Total protein extracts were prepared by lysing cells in extraction buffer (HEPES KOH [pH 8.5], NaCl 400 mM, EDTA 0.1 mM, EGTA 0.1 mM, DTT 1 mM, 1× protease inhibitor, SDS 1%). Proteins were separated by electrophoresis on BOLT 4%-12% bis-tris polyacrylamide precast gels in MES buffer (Life Technologies) and transferred on 0.2 μm nitrocellulose membrane. Non-specific signals were blocked with 5% Milk-PBS-Tween0.5% and the membrane hybridized overnight at 4° C. with primary and secondary antibodies diluted in blocking buffer (supplementary table S1). Horseradish peroxidase-conjugated secondary antibodies were revealed with the ECL chemiluminescence kit (Amersham) and signals detected with ChemiDoc (Bio-Rad).

### Mass Spectrometry Proteomic Analysis

Total protein extracts were prepared with RIPA buffer (50mMTris-cl pH 8.0, 5 mM EDTA, 150 mM Nacl, 15 mM Mgcl2, 1% NP-40, 1 mM PMSF and 1× Protease Inhibitor Cocktail (PIC)). Further, a sonication step was included: (30S ON, 30S OFF, 10 cycles with Bioruptor). Equal 50 mg protein extracts were concentrated to 30 ml volume, then diluted in 8M urea in 0.1M Tris-HCl, followed by protein digestion with trypsin, according to the FASP protocol(66). After an overnight digestion, the peptides were eluted from the filters with 25 mM ammonium bicarbonate buffer. The eluted peptides were processed in the desalting step by using Sep-Pag C18 Column (waters) based on the manufacture's instruction. Approximately 200 ng of peptide mixture per sample was analyzed using a timsTOF Pro 2 QTOF mass spectrometer coupled with a nanoElute liquid chromatography system (Bruker Daltonik GmbH, Germany). The sample was injected directly into a RP-C18 Aurora emitter column (75 μm i.d.×250 mm, 1.6 μm, 120 Å pore size) (Ion Opticks, Australia) using a one-column separation method. An 80-min gradient was established using mobile phase A (0.1% FA in H2O) and mobile phase B (0.1% FA in Acetonitrile): 2-25% B for 60 min, 25-37% for 10 min, ramping 37% to 95% in 5 min, and maintaining 95% B for 5 min. The column temperature was set at 50° C. and the flow rate at 250 nl/min. The sample eluting from the separation column was introduced into the mass spectrometer via a CaptiveSpray nano-electrospray ion source (Bruker Daltonik GmbH) with an electrospray voltage of 1.6 kV. The ion source temperature set to 180° C. and a dry gas of 3 l/min. The samples were analyzed using diaPASEF(67) consisting of 24 cycles including a total of 48 mass width windows (13 Da (m/z) from m/z 400 to 1,000 and TIMS scan range from 0.63 to 1.35 Vs cm-2 (1/K0). The collisional energy increased linearly from 20.01 eV at 0.6 (1/K0) to 52.00 eV at 1.35 Vs cm-2 (1/K0). The scan range for MS and MS/MS spectra was set to 100-1700 m/z. TIMS ramping time and accumulation time were set to 100 milliseconds. The diaPASEF data were analyzed by directDA approach using Spectronaut software (version 14) following manufacture instructions.

RNA Extraction and Real-Time qPCR

Total RNA was extracted from cells and tissues using RNeasy Plus mini kit with gDNA eliminator column (Qiagen), followed by cDNA synthesis using iScript Reverse Transcription Supermix for RT-PCR (Bio-Rad). Real-Time GPCR was performed using SsoAdvanced SYBR Green Supermix or iQ Multiplex Powermix (Bio-Rad). The primers and probes used in this experiments are shown in Table 2.

TABLE 2
Primers and probes used in qPCR.

| SEQ ID | | | |
|---|---|---|---|
| NO: | Target | Direction | Sequence |
| 8 | mouse Suv29H1 | forward | CTTTGCCACAAGAACCATCTGGG |
| 9 | mouse Suv29H1 | reverse | GCCAAAGTTGGAGTCCATTCGG |
| 10 | mouse Suv39H1 | forward | CTTTGCCACAAGAACCATCTGGG |
| 11 | mouse Suv39H1 | reverse | GCCAAAGTTGGAGTCCATTCGG |
| 12 | mouse p16 | forward | CGTGAACATGTTGTTGAGGC |
| 13 | mouse p16 | reverse | GCAGAAGAGCTGCTACGTGA |
| 14 | mouse p21 | forward | CGGTGTCAGAGTCTAGGGGA |
| 15 | mouse p21 | reverse | ATCACCAGGATTGGACATGG |
| 16 | mouse Atf3 | forward | CTCTGGCCGTTCTCTGGA |
| 17 | mouse Atf3 | reverse | GGTCGCACTGACTTCTGAGG |
| 18 | mouse Gadd45b | forward | CGGCCAAACTGATGAATGT |
| 19 | mouse Gadd45b | reverse | TCTGCAGAGCGATATCATCC |
| 20 | mouse Btg2 | forward | GCGAGCAGAGACTCAAGGTT |
| 21 | mouse Btg2 | reverse | TAGCCAGAACCTTTGGATGG |
| 22 | mouse Mmp13 | forward | TGATGAAACCTGGACAAGCA |
| 23 | mouse Mmp13 | reverse | GGTCCTTGGAGTGATCCAGA |
| 24 | mouse Il1a | forward | TCTATGATGCAAGCTATGGCTCA |
| 25 | mouse Il1a | reverse | CGGCTCTCCTTGAAGGTGA |
| 26 | mouse LINE-1 A-family | forward | TGAGCACTGAAACTCAGAGGAG |
| 27 | mouse LINE-1 A-family | reverse | GATTGTTCTTCTGGTGATTCTGTTA |
| 28 | mouse LINE-1 Gf-family | forward | TGCCCACTGAAACTAAGGAGA |
| 29 | mouse LINE-1 Gf-family | reverse | GCTTGTTCTTCAGGTGACTCTGT |
| 30 | mouse LINE-1 Tf-family | forward | ACAGACGTACCTTCCTCACC |
| 31 | mouse LINE-1 Tf-family | reverse | CCCCATCACACCCAAGAACA |
| 32 | mouse LINE-1 5′UTR | forward | CAGCCGGCCACCTTCC |
| 33 | mouse LINE-1 5′UTR | reverse | GGTCCCGGACCAAGATGG |
| 34 | mouse Gapdh | forward | CGGCCGCATCTTCTTGTG |
| 35 | mouse Gapdh | reverse | GTGACCAGGCGCCCAATA |
| 36 | mouse LINE-1 3′UTR | forward | AGACTGCCATAGCCAGGGATC |
| 37 | mouse LINE-1 3′UTR | reverse | CAGCTACATCTGCGTCCTTTCA |
| 38 | human Suv39H1 | forward | CCGCCTACTATGGCAACATCTC |
| 39 | human Suv39H1 | reverse | CTTGTGGCAAAGAAAGCGATGCG |
| 40 | human p16 | forward | CCCAACGCACCGAATAGTTA |
| 41 | human p16 | reverse | ACCAGCGTGTCCAGGAAG |
| 42 | human p21 | forward | TGGAGACTCTCAGGGTCGAAA |
| 43 | human p21 | reverse | GGCGTTTGGAGTGGTAGAAATC |
| 44 | human Atf3 | forward | TGCTCAGAGAAGTCGGAAGAA |
| 45 | human Atf3 | reverse | TGGCACAAAGTTCATAGGGCA |
| 46 | human Mmp13 | forward | ACTGAGAGGCTCCGAGAAATG |
| 47 | human Mmp13 | reverse | GAACCCCGCATCTTGGCTT |
| 48 | human Btg2 | forward | ACGGGAAGGGAACCGACAT |
| 49 | human Btg2 | reverse | ACGGGAAGGGAACCGACAT |
| 50 | Human LINE-1 Ta-family | forward | CAAACACCGCATATTCTCACTCA |
| 51 | Human LINE-1 Ta-family | reverse | CTTCCTGTGTCCATGTGATCTCA |
| 52 | human Gapdh | forward | TTGAGGTCAATGAAGGGGTC |
| 53 | human Gapdh | reverse | GAAGGTGAAGGTCGGAGTCA |
| 54 | Human chr1: 2611417- | forward | CCAGACCCCGTGTTTCCC |
| | 2907892 | | |
| 55 | Human chr1: 2611417- | reverse | GACAAGGACACCCAGGGC |
| | 2907892 | | |
| 56 | Human chr2: 95932366- | forward | AGCCCCTCTCACATCCCA |
| | 96791133 | | |
| 57 | Human chr2: 95932366- | reverse | GATGGCTTCAGGGTGGGG |
| | 96791133 | | |
| 58 | Human chr2: 240383252- | forward | GCTTGGCAGGTCCCATGT |
| | 241590562 | | |
| 59 | Human chr2: 240383252- | reverse | TGTGGCAGAGTCAGCAGC |
| | 241590562 | | |
| 60 | Human chr4: 610583- | forward | CGGCTTTGTGAGTCCCGT |
| | 2397676 | | |
| 61 | Human chr4: 610583- | reverse | GGCTTCACTGAGGCACGT |
| | 2397676 | | |
| 62 | Human chr6: 168770242- | forward | AAGACACACCTGCTCCGC |
| | 168797255 | | |
| 63 | Human chr6: 168770242- | reverse | TGGCTCACAGTTCCGCAG |
| | 168797255 | | |
| 64 | Human chr6: 170244713- | forward | TGAAGGTCACAGGCTGCG |
| | 170485862 | | |
| 65 | Human chr6: 170244713- | reverse | CCACGCTGAGGTTGCAGA |
| | 170485862 | | |
| 66 | Human chr7: 383506- | forward | CCTGGCGCCTTCCTGAAA |
| | 743563 | | |
| 67 | Human chr7: 383506- | reverse | GCGTGCAGTTCCCTCACT |
| | 743563 | | |
| 68 | Human chr7: 27178748- | forward | CTGTTCGGCTGCCCAGAA |
| | 27233348 | | |
| 69 | Human chr7: 27178748- | reverse | GTCTGGCCGGAAAACCGA |
| | 27233348 | | |
| 70 | Human chr8: 47000524- | forward | CACCCCCTCCCACCCATA |
| | 47342037 | | |
| 71 | Human chr8: 47000524- | reverse | TCAAAACCACCTCCGCCC |
| | 47342037 | | |
| 72 | Human chr8: 146053793- | forward | CTGTCTGCCACCACCCAG |
| | 146077272 | | |
| 73 | Human chr8: 146053793- | reverse | GAAGGCAACCACCGAGCT |
| | 146077272 | | |
| 74 | Human chr10: 102488677- | forward | ATCCACACCACCAAGCCG |
| | 103002682 | | |
| 75 | Human_chr10: 102488677- | reverse | GGCTGTGGGGATTGGCTT |
| | 103002682 | | |
| 76 | Human chr11: 2828602- | forward | CAGGGCAGTGACAGGGTG |
| | 2855961 | | |
| 77 | Human chr11: 2828602- | reverse | ATGGTGGTGCGTCCTTGG |
| | 2855961 | | |
| 78 | Human chr12: | forward | GCAGAAGGGACGGGGAAC |
| | 132877550-133814303 | | |
| 79 | Human chr12: | reverse | GTCCCCACGTGCACTACC |
| | 132877550-133814303 | | |
| 80 | Human chr14: | forward | GCATGATGGTGGGTGCCT |
| | 106053443-106388110 | | |
| 81 | Human chr14: | reverse | CAGTCACACGGGCTGGAG |
| | 106053443-106388110 | | |
| 82 | Human chr15: 25292601- | forward | ACGCCCACTGAAGGAAGC |
| | 25501678 | | |
| 83 | Human chr15: 25292601- | reverse | TTGCTCAGGATCGCTGGC |
| | 25501678 | | |
| 84 | Human chr16: 884547- | forward | GTGGACACAGGGGAAGGC |
| | 910725 | | |
| 85 | Human chr16: 884547- | reverse | GTGTTTCTGGGGGACGGG |
| | 910725 | | |
| 86 | Human chr17: 36714636- | forward | CAGGCAGGGTGGAAGGTG |
| | 36736745 | | |
| 87 | Human chr17: 36714636- | reverse | GGAGGGGGATGAGGACGT |
| | 36736745 | | |
| 88 | Human chr19: 19780869- | forward | ACAGCCACAGCCACTTCC |
| | 19869411 | | |
| 89 | Human chr19: 19780869- | reverse | CTGTGGCCCTGTGACCTG |
| | 19869411 | | |
| 90 | Human chr22: 48981410- | forward | GAGGGAGGAAGGGGAGGG |
| | 49564632 | | |
| 91 | Human chr22: 48981410- | reverse | ACGTGAGGTAGGGCGAGT |
| | 49564632 | | |
| 92 | Human chrX: 58279640- | forward | GCCTGCCTTGACCTCTCC |
| | 58319234 | | |
| 93 | Human chrX: 58279640- | reverse | GGTGCTCTGCCGTAGTCC |
| | 58319234 | | |
| | mouse LINE-1 A-family | probe | GAATCTGTCTCCCAGGTCTG |
| | mouse LINE-1 Gf-family | probe | TGCTACCCTCCAGGTCTGCT |
| | mouse LINE-1 Tf-family | probe | AAAAAGAGCCCTCCTTGTGC |
| | mouse LINE-1 5′UTR | probe | CGGAGGACAGGTGC |
| | mouse LINE-1 3′UTR | probe | TGACACCATTGCATACACT |
| | Human LINE-1 Ta-family | probe | AGGTGGGAATTGAAC |

### RNA-Seq and Data Analysis

Total RNA-seq library was prepared with CORALL Total RNA library prep with RiboCop rRNA for Human/Mouse/Rat depletion kit (Lexogen GmbH, Vienna, Austria) following manufacturer's instructions (library type: fr-secondstrand) by IGA Technology service (Italy). Final libraries were checked with both Qubit 2.0 Fluorometer (Invitrogen, Carlsbad, CA) and Agilent Bioanalyzer DNA assay or Caliper (PerkinElmer, Waltham, MA). Libraries were then prepared for sequencing and sequenced on paired-end 150 bp mode on NovaSeq6000 (Illumina, San Diego, CA). RNA-seq read quality control (QC) analyses and filtering of high-quality reads were executed using FastQC and BBDuk by setting a minimum read length of 35 bp and a minimum Phred-quality score of 25. After trimming quality control, high-quality reads were aligned to the mouse genome reference (GrCm38p6/mm10) with STAR 2.7.3a, while FeatureCounts 1.6.3 package was used to assign reads to genes. Next, Genes with low, constant levels of expression across one or more experimental conditions were filtered to eliminate the “uninformative” genes using HTSFilter 1.30.1. Filtered gene data were further processed with the EdgeR package to normalize (Trimmed Mean of M-values, TMM, method) the raw counts and perform differential gene expression analysis. TMM normalized counts were subjected to Gene Set Enrichment Analysis using the GSEA 4.1.0 software, a joint project of UC San Diego and Broad Institute (http://software.broadinstitute.org/cancer/software/gsea/wiki/index.php/Main_Page). The number of permutations was adjusted to 1000, and a custom gene sets database was constructed using Gene Sets related to senescence, aging or progeria from the GSEA Molecular Signatures Database (http://www.gsea-msigdb.org/gsea/msigdb/geneset_page.jsp). Expression of Interspersed Repeat elements was quantified using SQuiRE 0.9.9.92 (https.//github.com/wyang17/SquIRE). SQuiRE provides locus-specific expression quantification along with subfamily-level expression estimates counting unambiguously mapped reads, as well as ambiguously mapped reads using an expectation-maximization (EM) algorithm (68). When comparing the differentially expressed genes between the three experimental groups (WT vs HGPS or LAKI and HGPS or LAKI vs L1-ASO treated samples), percentage of recovered genes was defined as the percentage of differentially expressed genes in both HGPS/LAKI Vs WT and HGPS/LAKI L1-ASO Vs Scr-ASO treated samples from the total number of differentially expressed genes (DESeq2 FDR cut-off <0.05).

### Senescence-Associated Beta-Galactosidase Enzymatic Activity Assay

Senescence-associated beta-galactosidase (SA-3-gal) assay was performed as previously described in ref.(69). Briefly, first, the cells were fixed in 4% paraformaldehyde for 5 min at room temperature. Next, the cells were washed twice with PBS and incubated overnight 37° C. in a staining solution containing 40 mM citric acid/Na phosphate buffer, 5 mM K4[Fe(CN)6]3H2O, 5 mM K3[Fe(CN)6], 150 mM sodium chloride, 2 mM magnesium chloride and 1 mg/ml X-gal. Finally, the cells were washed twice with PBS and once with methanol. The plate was dried, and photos of cells were taken using bright-field microscopy.

### Elisa Kits

The following ELISA kits were used in this work: H3K9me3 quantification kit, order no. #P-3035-96 from Epigentek; H3K27me3 quantification kit, order no. #P-3020-T from Epigentek; pH2AX quantification kit, order no. #50929C from Cell Signaling Technology

### Antisense Oligonucleotides (ASOs)

The ASOs used in the working examples are shown in Table 3. Table 3. ASOs.

| SEQ ID | ASO | | |
|---|---|---|---|
| NO: | name | Sequence | Source |
| 1 | human L1 | A*C*T* T*C*C CTT CTC GCT TC*A*T*T*T* | AUMbiotech |
| | 2′F-ANA | | |
| | ASO #1 | | |
| 2 | mouse L1 | T*T*G* A*C*C TTT CTC CCT TA*C T*G*C* | AUMbiotech |
| | 2′F-ANA | | |
| | ASO #1 | | |
| 3 | mouse L1 | A*T*A* T*G*T TAC TTG ACC T*T*T* C*T*C | AUMbiotech |
| | 2′F-ANA | | |
| | ASO #2 | | |
| 4 | mouse L1 | T*G*T* A*A*T TCT GAT AGG CC*T* | AUMbiotech |
| | 2′F-ANA | T*C*C* | |
| | ASO #3 | | |
| 5 | mouse L1 | A*G*T* G*TC TGT ATA ACA TCT *G*T*C* | AUMbiotech |
| | 2′F-ANA | | |
| | ASO #4 | | |
| 6 | Human | C*AA*AG*AT*GG*GG*AA*AA*AA*CA*GA* | Qiagen/Exiqon |
| | LINE-1 | | |
| | translation | | |
| | blocker | | |
| | LNA | | |
| | ASO #1 | | |
| 7 | human | A*AC*AT*CA*TA*AT*GA*CA*GG*AT*CA* | Qiagen/Exiqon |
| | LINE-1 | | |
| | translation | | |
| | blocker | | |
| | LNA | | |
| | ASO#2 | | |

All ASOs were used in vitro. Mouse L1 2′F-ANA ASO #4 was used in vivo.

In 2′F-ANA ASOs, asterix (*) denotes 2′F-ANA modified nucleotides. In translation blocker ASOs, asterix (*) denotes modified nucleotides.

### Data Deposits

RNA deep sequencing data reported herein have been submitted to the NCBI short read archive (https://www.ncbi.nlm.nih.gov/sra) under the accession number PRJNA704498 and GSE198675. Proteomics data have been submitted to PRIDE database under the accession number PXD032944. DNA methylation data have been deposited in the GEO database under the accession number GSE200152.

### Example 2—L1 Upregulation is a Common Phenomenon Among Human Progeroid Syndromes

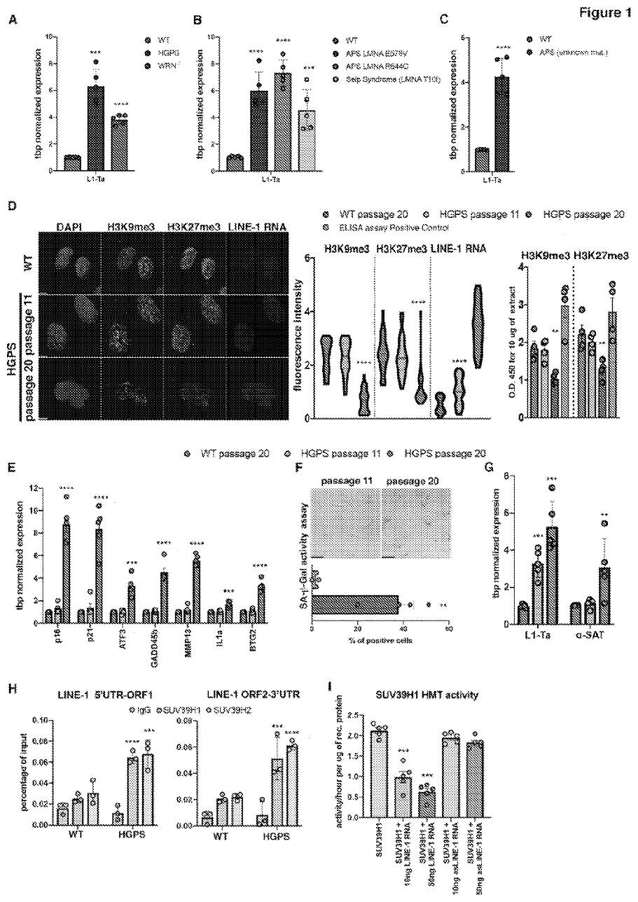

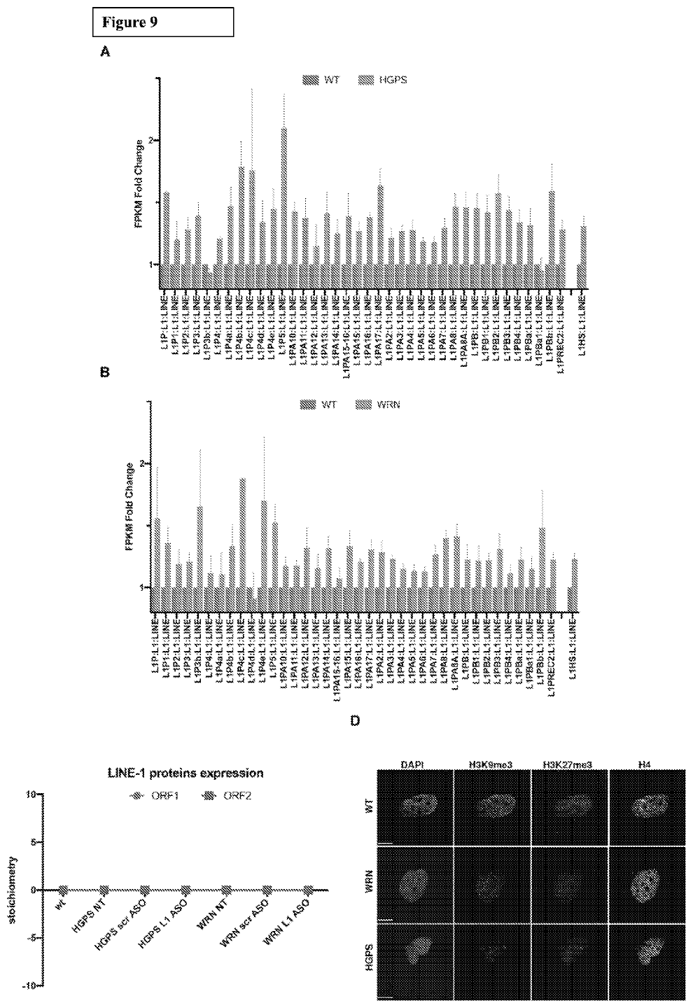

L1 activation is a common trait seen during physiological aging in all eukaryotic organisms (15). To determine whether the changes in L1 RNA expression are also associated with pathological aging and understand their role in driving aging, we focused on premature aging progeroid syndromes, both early- and late-onset. We initially measured the expression of LINE-1 elements—Transcribed subset a (L1-Ta) elements using a multiplexed TaqMan assay in human mesenchymal stem cells (hMSCs) differentiated from HGPS and WRN patient-derived pluripotent stem cells phenocopying the progeroid syndromes (8). We observed an increase in L1 expression in both the syndromes (FIG. 1A). To have a broader picture of L1 expression in HGPS and WRN, we performed RNA sequencing (RNA-seq) and used SQuIRE (Software for Quantifying Interspersed Repeat Expression) for the analysis of L1 expression (16). Consistent with the qPCR data, RNA-seq revealed that Homo sapiens-specific L1 (L1Hs) elements and most of the primate-specific L1 elements (L1P) were upregulated in HGPS and WRN (FIGS. 9, A and B). To determine whether upregulation of L1 expression is a common phenomenon among other progeroid syndromes, we analyzed L1 expression in primary fibroblasts derived from patients affected by different atypical progeroid syndromes (APS) (17, 18), driven by mutations in lamin A/C (LMNA) gene (which do not accumulate progerin) and by unknown mutations (19). Similar to HGPS and WRN, L1 expression was 4-7 times higher in the atypical progeroid syndrome cells compared to healthy donor control cells (FIGS. 1, B and C). These results demonstrated that L1 deregulation was commonly dysregulated among the different progeroid syndromes. In previous reports, during normal aging, L1-encoded proteins were detected (1, 12). We therefore applied mass spectrometry as described in Ardeljan et al. (20) to detect and quantify L1 open reading frame (ORF1 and ORF2) proteins. However, in wild type (WT), HGPS and WRN cells, both ORF1 and ORF2 proteins were undetectable (FIG. 9C, data not shown).

### Example 3—LINE-1 were Transcriptionally Upregulated During Chronological Aging in Healthy Human Cells

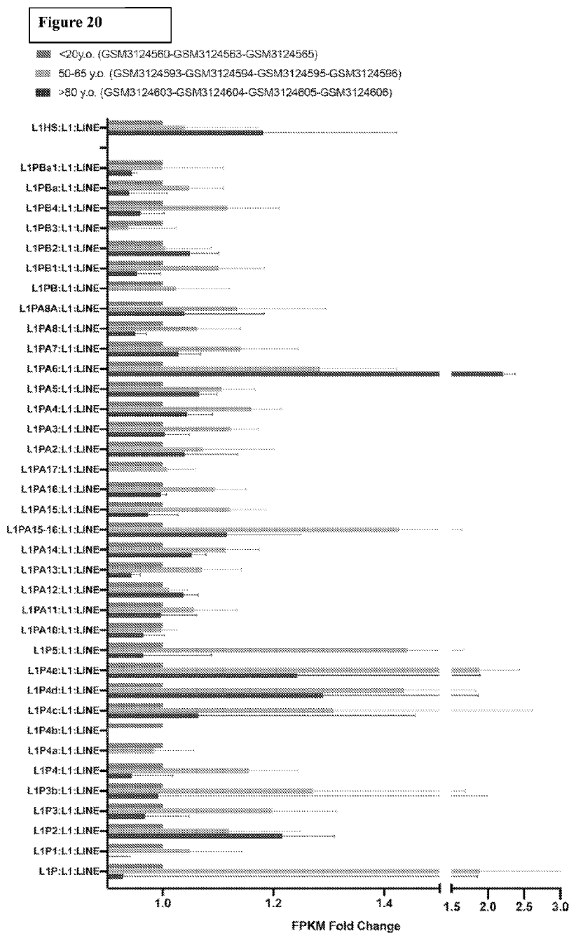

To have a broader picture of L1 expression in aging human cells, we performed RNA sequencing (RNA-seq) and used SQuIRE (Software for Quantifying Interspersed Repeat Expression) for the analysis of L1 expression. RNA-seq revealed that Homo sapiens-specific L1 (L1Hs) elements and primate-specific L1 elements (LIP) were transcriptionally upregulated in human cells from healthy donors of different ages (FIG. 20).

### Example 4—L1 Deregulation was an Early Event and Preceded Both Loss of Heterochromatin and Onset of a Senescence Phenotype

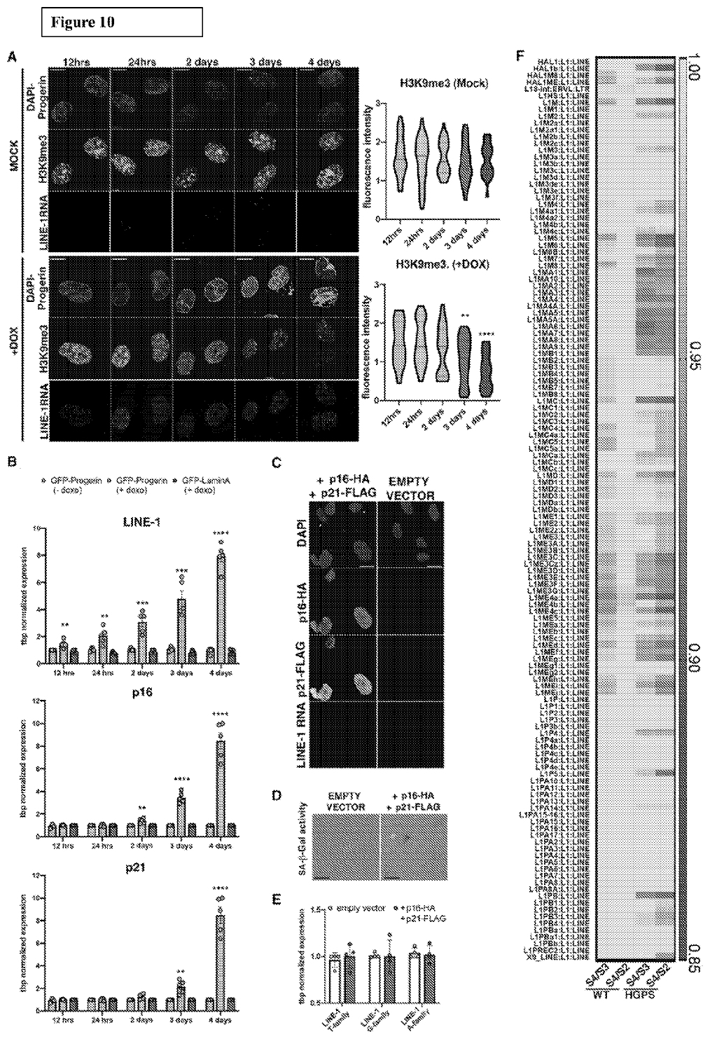

The correlation between repetitive elements expression and an aged phenotype has been extensively characterized in eukaryotic organisms (9, 15, 21, 22). However, it is still unknown if this is a consequence of the genetic drift caused by senescence or if L1 RNA has an active role in aging progression. In the eukaryotic genome, L1 elements, like most of the interspersed repetitive sequences, are tightly repressed by nuclear lamina-associated histone 3 lysine 9 trimethylation (H3K9me3) constitutive heterochromatin domains (23-26). Sequential analysis of macromolecules accessibility (SAMMY-seq), a method used to sequence nuclear matrix-interacting DNA, was recently applied to study aberrant organization and chromatin accessibility of the heterochromatinized DNA, usually compacted at the nuclear lamina, in primary HGPS fibroblasts (27). In that study, the increased heterochromatin accessibility, caused by LMNA mutation, occurred before the loss of H3K9me3 and histone 3 lysine 27 trimethylation (H3K27me3) and led to transcriptional instability and the onset of senescent phenotypes (27). Therefore, we measured the amounts of H3K9me3, H3K27me3, the expression of senescence-associated secretory phenotype (SASP) genes and L1 in early and late passage HGPS cells (FIG. 1, D to G, FIG. 9D). Consistent with the previous study, early passage HGPS cells (passage 11) showed neither change in the amounts of H3K9me3 or H3K27me3 (FIG. 1D), SASP genes such as: p16, p21, Activating Transcription Factor 3 (ATF3), Matrix Metallopeptidase 13 (MMP13), Interleukin 1a (IL1a), BTG Anti-Proliferation Factor 2 (BTG2) and Growth Arrest and DNA Damage Inducible Beta (GADD45b) (FIG. 1E) and positivity for senescence-associated β-galactosidase enzyme (SA-3-gal) activity (FIG. 1F), compared to healthy control cells. In early passage cells, L1 elements, and no other repetitive sequences typically marked by H3K9me3-like satellite DNA, were overexpressed, suggesting that L1 RNA aberrant expression preceded heterochromatin defects (FIG. 1G). To corroborate the evidence that L1 expression precedes both epigenetic defects and the onset of the senescent phenotype, we took advantage of human fibroblasts cell line with inducible green fluorescent protein (GFP)-Progerin cassette that expresses progerin in the presence of doxycycline and recapitulates both the phenotypic (5) and epigenetic defects (28) observed in cells from patients with HGPS. We monitored L1 RNA expression in these inducible GFP-Progerin fibroblasts collected at 12 hr, 24 hr, 2, 3, and 4 days after doxycycline induction. L1 element overexpression was detected after 12 hours of doxycycline treatment, although heterochromatin erosion and p16 and p21 gene expression were not observed until day 2 (FIGS. 10, A and B). Furthermore, L1 expression was not observed during overexpression of p16-hemagglutinin (HA) and p21-FLAG in normal fibroblasts (FIG. 10, C to E). Reanalysis of available SAMMY-seq datasets (short read archive (SRA) accession number: PRJNA483177) on WT and early passage HGPS cells showed a shift of L1 DNA from the nuclear matrix-associated chromatin fraction (S4 fraction, yellow color) to the more accessible and pervasively transcribed chromatin (S3 and S2 fractions, blue) (FIG. 10F). These results suggested that L1 dysregulation is an early event that eventually leads to the loss of heterochromatin and the expression of SASP genes.

L1 RNA, like other repetitive RNAs, is able to interact with the H3K9 histone methyltransferase SUV39 (29). We therefore performed f-RIP (RNA immunoprecipitation on fixed cells) to assess if L1 RNA interacted with SUV39H1 and SUV39H2 proteins. The immunoprecipitation assay showed increased interaction of both SUV39 isoforms in HGPS cells compared to healthy controls (FIG. 1H). Further, we tested the effect of this interaction by measuring the enzymatic activity of SUV39H1. According to the gene-tissue expression (Gtex) program, the SUV39H1 isoform is more abundant in normal somatic tissues, whereas SUV39H2 is mostly expressed in the testes and embryonic stem cells [https://www.gtexportal.org/home/gene/SUV39H2 and (21)]. An ELISA-based SUV39 enzymatic activity test was performed by incubating SUV39H1 with 10 ng and 50 ng of L1 RNA using antisense L1 RNA as control. Both concentrations of L1 RNA exerted an inhibitory effect on SUV39H1 activity (FIG. 1I), demonstrating that high L1 RNA expression is sufficient to inhibit SUV39H1 activity.

### Example 5—L1 RNA Depletion Ameliorated the Senescent Phenotype in Human Progeroid Syndromes

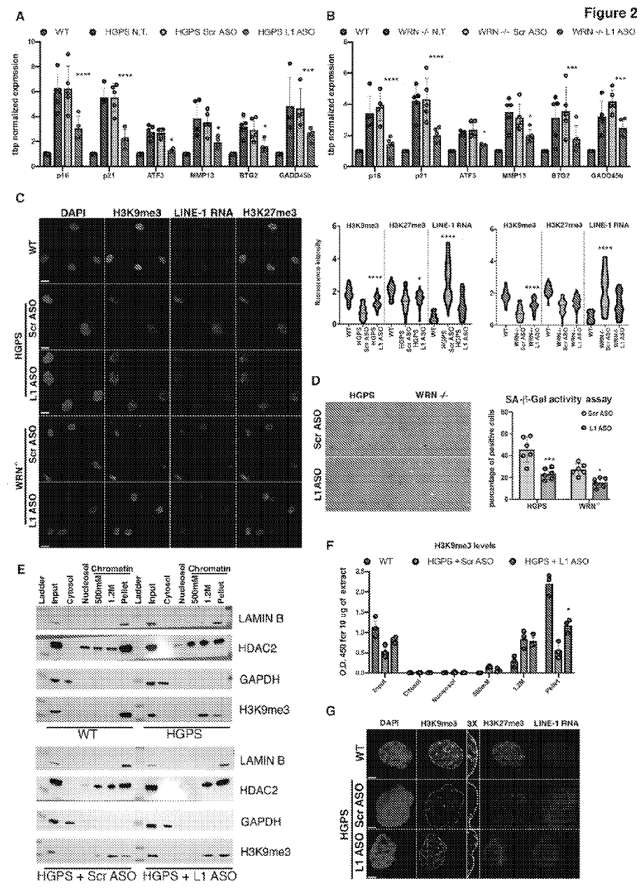

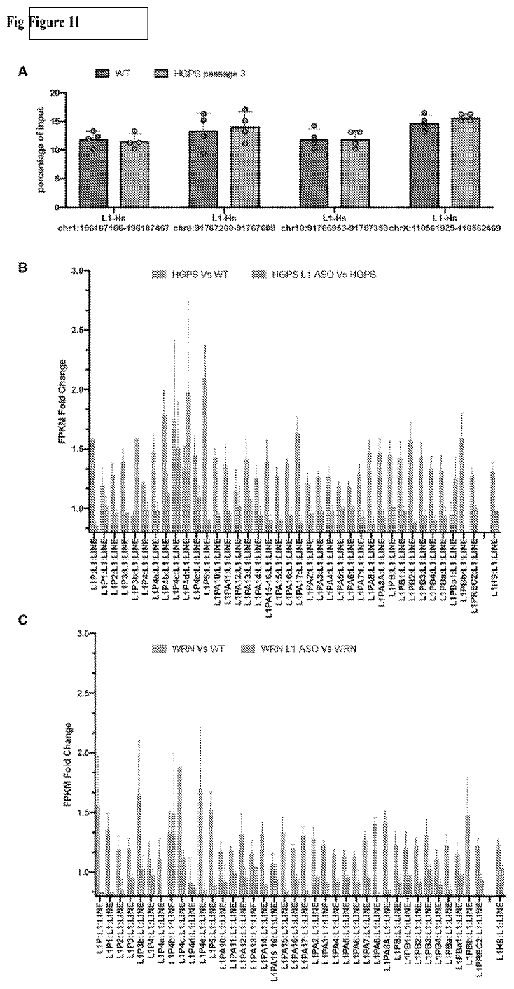

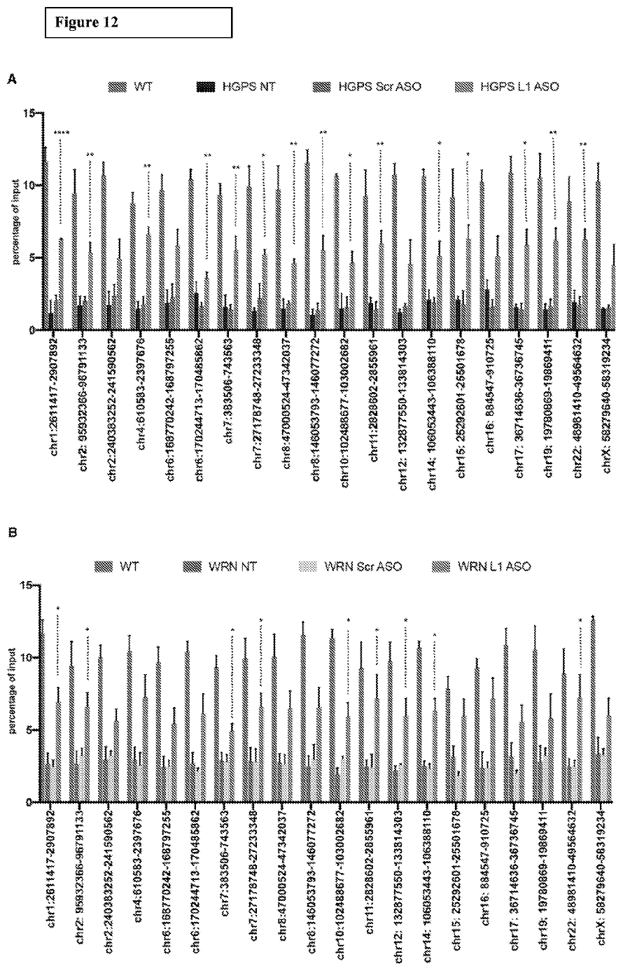

To evaluate the effects of depletion of L1 RNA aberrantly accumulated in the nucleus, we used a L1-specific 2′F-ANA (2′-deoxy-2′-fluoro-D-arabinonucleic acid) modified ASO (L1 ASO) to knockdown L1 RNA (30). The sequence of the ASO (termed “human L1 2′F-ANA ASO #1”) is ACTTCCCTTCTCGCTTCATTT (SEQ ID NO: X). Location of 2′F-ANA modifications are shown in Table 3. This ASO was used in all human cell experiments described in the Examples. The 2′F-ANA modification in the oligonucleotide backbone improves both cell permeability and the stability of the oligonucleotides inside the cells compared to non-modified ASOs (31, 32). RNA-seq analysis using SQuIRE confirmed downregulation of L1 Homo sapiens (L1HS) elements and most of L1 primate-specific (L1P) elements even with no variation of H3K9me3. (FIG. 11, A to C). L1 ASO prevented the expression of SASP genes (p16, p21, ATF3, MMP13, BTG2 and GADD45b) in HGPS and WRN hMSCs (FIGS. 2, A and B). Moreover, the constitutive heterochromatin mark H3K9me3, and to some extent, the facultative heterochromatin mark H3K27me3, increased upon L1 RNA depletion in HGPS and WRN MSCs (FIG. 2C). Zhang et al. previously identified specific H3K9me3 domains eroded in accelerated aging syndromes (8). We therefore analyzed H3K9me3 enrichment at these specific loci in scrambled (Scr) ASO and L1 ASO-treated HGPS and WRN cells with non-treated and WT cells. L1 ASO treatment reduced H3K9me3 loss in most of the loci in both HGPS and WRN cells (FIGS. 12, A and B). L1 downregulation reduced the numbers of SA-β-Gal-positive cells in both HGPS and WRN MSCs (FIG. 2D).

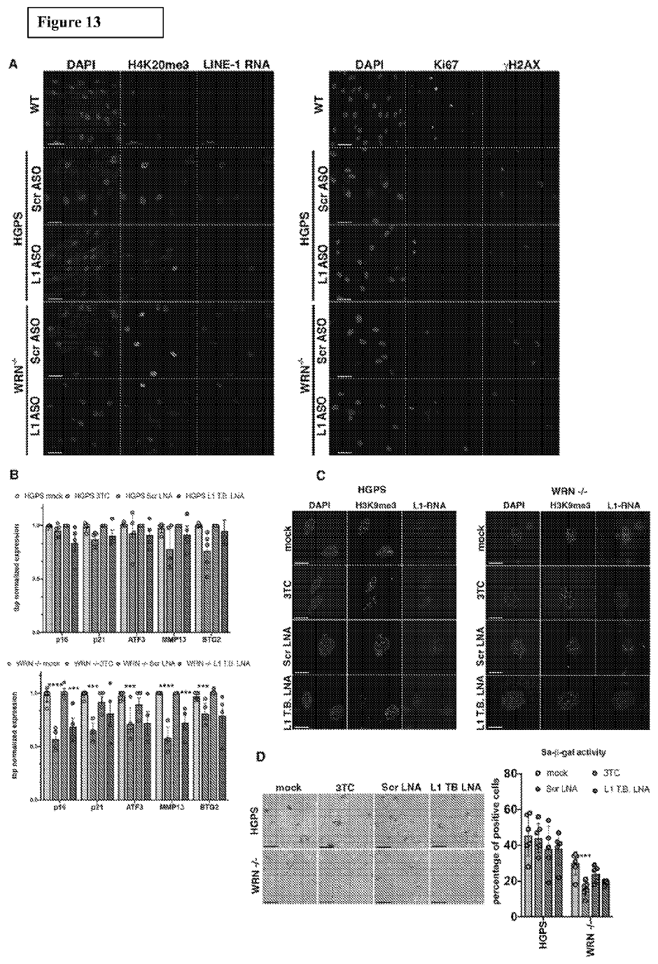

Next, it was investigated if the increased H3K9me3 marks were associated with the re-localization of heterochromatin at the nuclear matrix. Chromatin fractionation assays were performed in WT, HGPS, and HGPS L1 ASO-treated cells by isolating proteins from the cytosol and nucleosol fractions, the low and high-affinity chromatin-bound proteins, and the proteins associated with the nuclear matrix (33, 34). In agreement with a recent study (27), in HGPS cells H3K9me3 shifted from the nuclear matrix compartment to the less compacted and accessible chromatin fraction (FIG. 2E). In L1 ASO-treated HGPS fibroblasts, H3K9me3 marks increased in the nuclear matrix compartment, leading to heterochromatin compartmentalization similar to WT cells (FIG. 2E). The amounts of H3K9me3 in all the fractions used for the SDS-PAGE experiments have been quantified by ELISA assay (FIG. 2F). This was also confirmed by H3K9me3 immunofluorescence, where heterochromatin foci localization at the nuclei border (white dotted line) increased upon L1 ASO treatment (FIG. 2G). L1 ASO treatment also reduced H4K20me3, a repressive histone mark usually accumulated on chromatin in accelerated aging syndromes (35, 36) (FIG. 13A, left panel). Furthermore, L1 ASO treatment increased cell proliferation (Ki67 positive cells) and reduced the number of cells with gamma H2A histone family member X (γH2AX) signal in HGPS and WRN hMSCs (FIG. 13A, right panel).

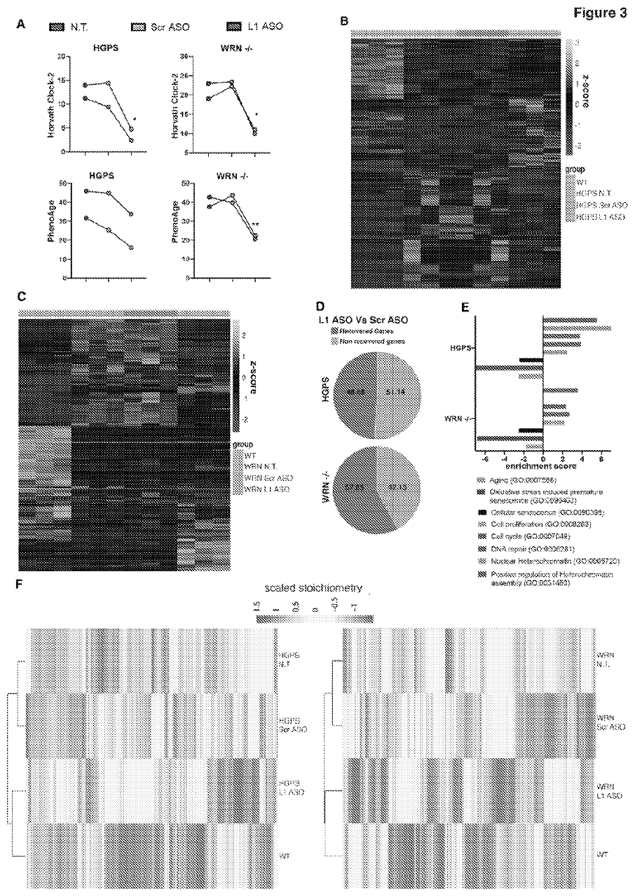

DNA methylation can be used to build pan-tissue estimators of age and mortality risk (epigenetic clocks) (37-39). Epigenetic clock studies have previously shown that fibroblasts from HGPS patients and blood samples from WRN patients exhibit an accelerated DNA methylation age (38, 40). DNA methylation analysis of L1 ASO-treated HGPS and WRN cells indicated a reduced DNA methylation age (FIG. 3A).

Moreover, differential gene expression analysis of RNA-seq data showed a recovery of gene expression in L1 ASO-treated cells compared to both non-treated and Scr ASO-treated HGPS and WRN cells (FIG. 3, B to E). We further investigated the effects of the L1 ASO on the proteome by quantitative mass spectrometry. Consistent with DNA methylation and RNA-seq analyses, the hierarchical clustering of protein differential expression analysis based on quantitative mass spectrometry in WT, HGPS and WRN fibroblasts demonstrated that L1-ASO treatment ameliorated the aged phenotype also at proteome global level (FIG. 3F).

We compared the effect of L1 RNA depletion using ASO with 3TC-induced L1 ORF2 enzymatic inhibition and antisense Locked Nucleic Acid (LNA) GapmeR-induced L1 RNA translation block (L1 T.B.) in HGPS and WRN hMSCs. Consistent with the undetectability of L1 ORF2 protein by mass spectrometry (FIG. 9C), in HGPS cells, 3TC and L1 T.B had no effect. In WRN cells, 3TC had a less potent anti-aging effect compared to L1 RNA depletion. Similarly, no effects of L1 T.B. were observed in WRN cells (FIG. 13, B to D). These results suggested that L1 RNA accumulated in the nucleus of HGPS and WRN cells, and not the cDNA, led to the manifestation of aging phenotypes, which can be rescued by L1 ASO but not with the reverse transcriptase inhibitor, 3TC.

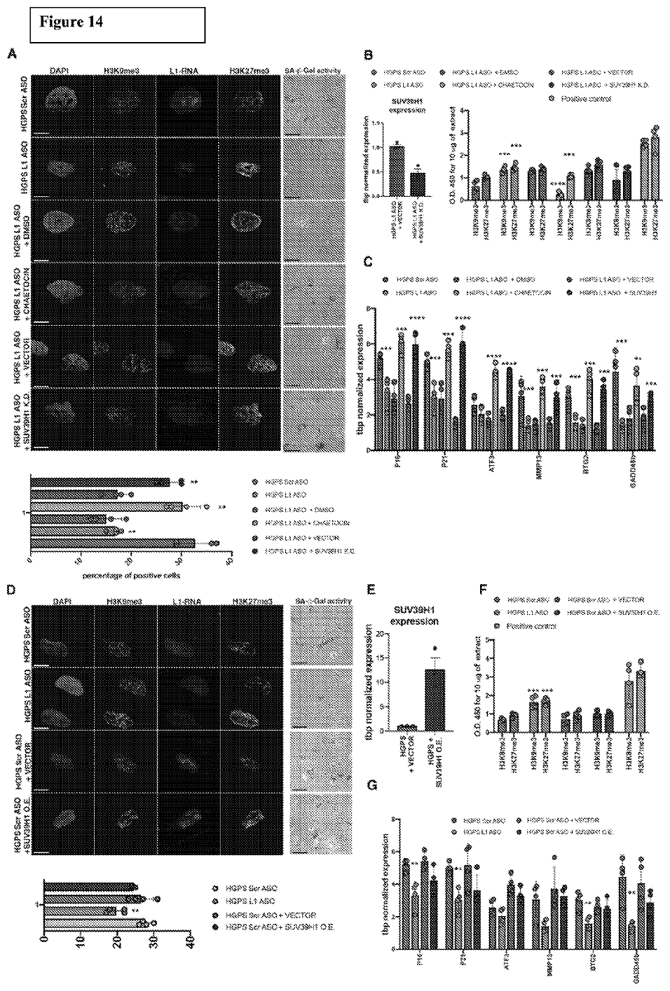

It was then tested whether L1 RNA silencing maintains its beneficial effect in the absence of SUV39H1 enzymatic activity. Both Chaetocin-mediated SUV39H1 inhibition and shRNA-mediated SUV39H1 knockdown abrogated the anti-aging effects of L1 ASO treatment, measured as H3K9me3 loss, prevention of the SASP genes expression and accumulation of SA-β-Gal-positive cells (FIG. 14, A to C). Further, SUV39H1 overexpression show partial anti-aging effects when compared to L1 ASO treatment (FIG. 14, D to G).

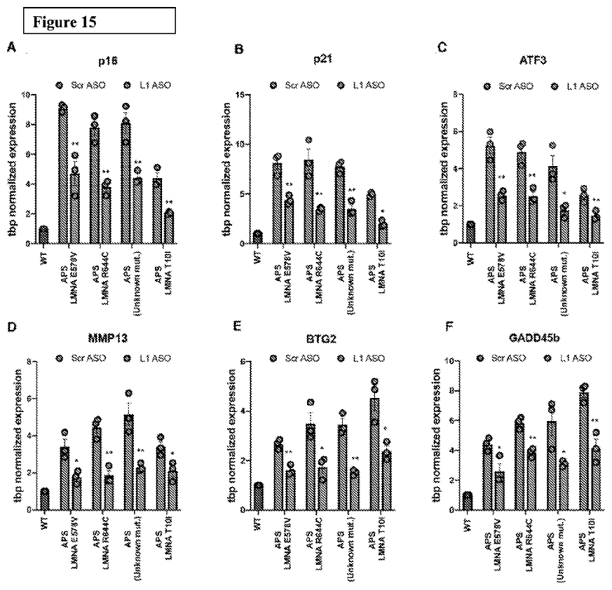

To further explore whether L1 ASO has rescue effects in other progeroid syndromes with L1 RNA overexpression, we treated the atypical progeroid syndrome cells analyzed previously with L1 ASO. L1 ASO reduced the expression of SASP genes in other progeroid syndrome patient-derived cells (FIG. 15, A to F). These results demonstrated that L1 ASO was broadly effective in reverting the aging hallmarks in vitro in different progeroid syndromes, especially the ones without progerin accumulation or mutations in LMNA gene, where farnesylation inhibitors or exon skipping strategy are not applicable (41, 42).

### Example 6—L1 RNA Depletion Restored H3K9Me3 and Reduced the Expression of Senescence-Associated Genes in Fibroblasts Derived from LAKI Mice

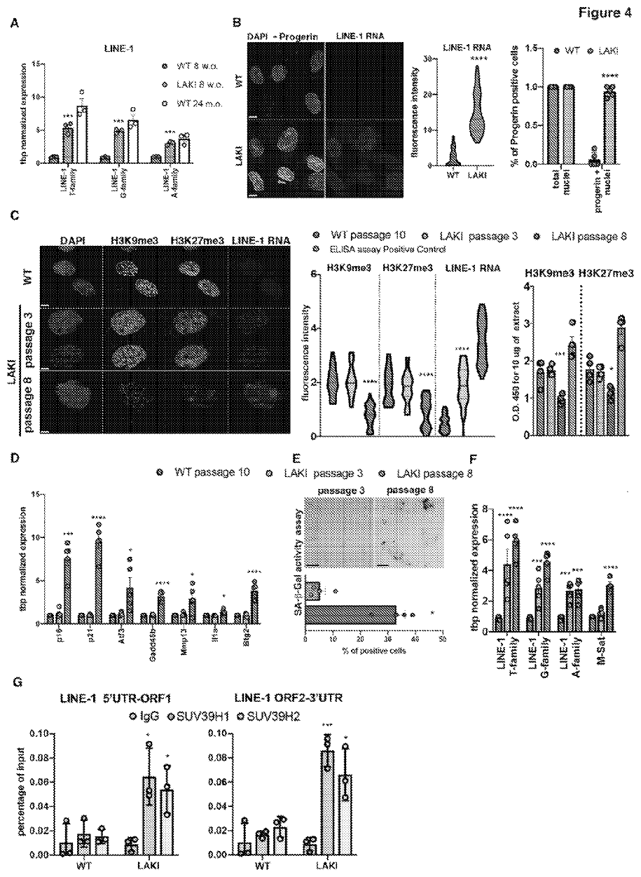

Upregulation of L1 RNA was assesssed in a premature aging mouse model, with a G609G mutation knocked into the gene Lmna (LAKI). Using a multiplexed TaqMan assay, we measured the expression of the three active murine L1 subfamilies (L1-Tf, L1-Gf, and L1-Af) in tail-tip fibroblasts (TTFs) isolated from wild-type (WT) and LAKI mice. In LAKI TTFs, a 3 to 6 times higher expression of L1 elements was observed, similar to senescent TTFs isolated from 24-month-old WT mice (FIG. 4A). We confirmed increased L1 expression and noticed the strong accumulation of L1 RNA only inside the nucleus (FIG. 4B). Similar to HGPS cells, LAKI TTFs at passage 3 (early passage) show no change in H3K9me3 and H3K27me3 (FIG. 4C), expression of SASP genes (FIG. 4D) and very low SA-3-Gal activity (FIG. 4E). However, L1 elements were deregulated in LAKI TTFs at passage 3 with 2-4 times higher expression than WT cells (FIG. 4F). Moreover, LAKI TTFs also recapitulated the same aberrant interaction between L1 RNA and Suv39 isoforms (FIG. 4G).

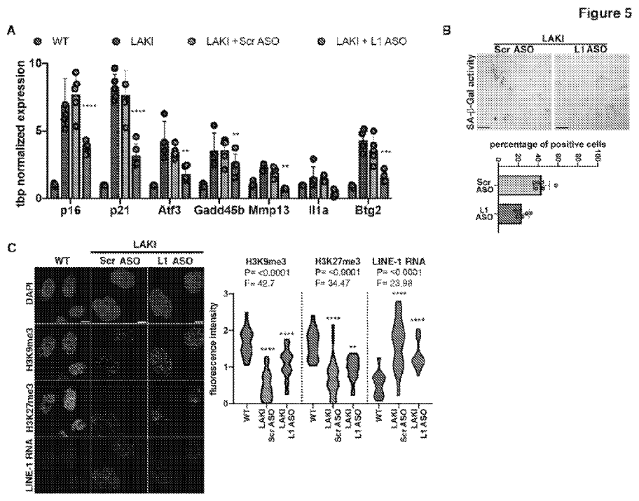

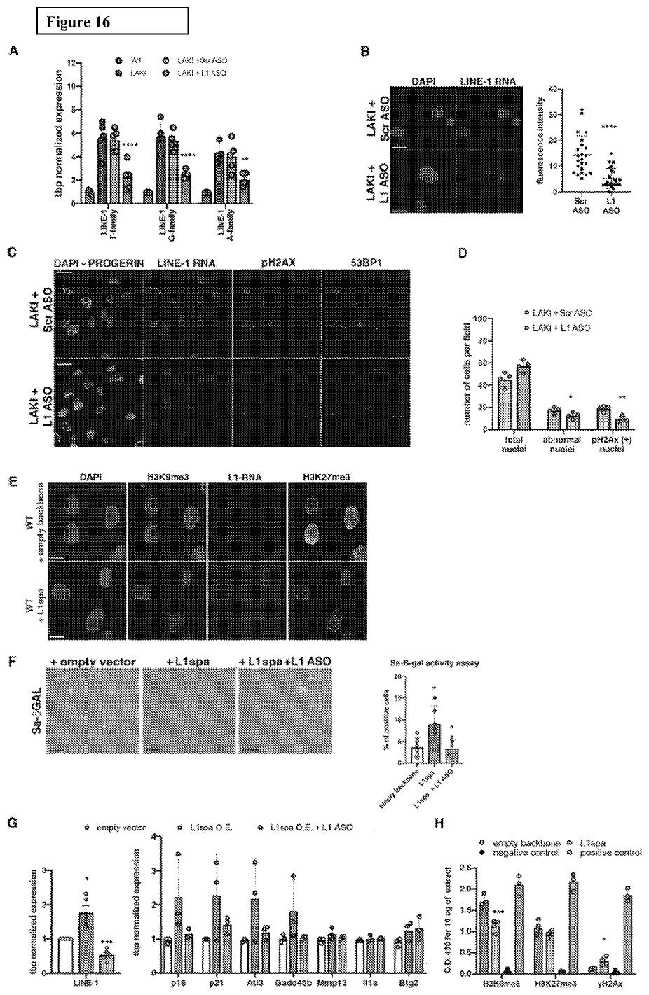

To knockdown L1 RNA in LAKI TTFs, we selected a mouse-specific L1 ASO (mouse L1 2′F-ANA ASO #4, listed in Table 3) targeting all L1-Tf, L1-Gf, and L1-Af elements. L1 RNA depletion was confirmed by qPCR and RNA FISH (FIG. 16, A and B). LAKI TTFs treated with L1 ASO showed a lower expression of stress response genes in the p53 tumor suppressor pathway (p16, p21, Atf3, and Gadd45b), senescence-associated metalloprotease Mmp13 and proinflammatory interleukin IL1a (FIG. 5A). The number of cells positive for active senescence-associated β-galactosidase enzyme (SA-β-gal), pH2Ax-53BP1 DNA damage foci, and abnormal nuclei were reduced in LAKI TTFs treated with L1 ASO (FIG. 5B and FIGS. 16, C and D). LAKI mice are characterized by low amounts of H3K9me3 and H3K27me3, as well as decondensed constitutive and facultative heterochromatin (7). Consistent with the data in human primary cells, the intensity of H3K9me3 constitutive heterochromatin foci and H3K27me3 facultative heterochromatin domains increased in LAKI cells after L1 ASO treatment compared to Scr ASO-treated control cells and was close to the quantity observed in WT cells (FIG. 5C).

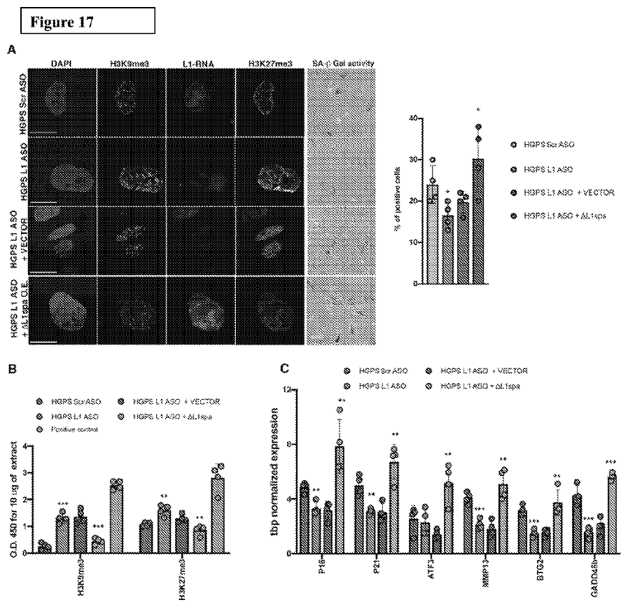

Transient transfection of WT TTFs with an active L1 element (L1spa) (Table 4) increased the proportion of SA-β-gal positive cells, induced the expression of SASP genes, decreased H3K9me3 marks and increased pH2Ax (FIG. 16, E to H). Inside the L1spa element, L1 ASO seeding sequence is flanked by SapI and PstI restriction sites. Using these two restriction enzymes, we generated a mutant L1spa element (ΔL1spa) resistant to L1 ASO (Table 4). ΔL1spa element overexpression in LAKI TTFs previously treated with L1 ASO overrode the protective effect of the L1 ASO, restarting heterochromatin erosion, upregulation of age-associated genes and accumulation of senescent SA-β-gal positive cells (FIG. 17 A to C). Collectively, these results demonstrated that murine-specific L1 ASOs can successfully knock down the aberrantly expressed L1 RNA in LAKI TTFs, restore healthy histone marks quantity and reduce the senescence-associated cellular phenotypes.

TABLE 4

| SEQ ID | | |
|---|---|---|
| NO | Name | Sequence |
| 94 | L1spa | ctttatagtattttgtacagttgccatattcgatatcctcctttttaatatcaaattgag |
| | | gtaaagcaattttttccaactgaacaggatctcctgactgccagatgaatcttaaatcat |
| | | cggttgtatatccaactacaataagtaaataaacaagttagaggaaaatatgcatatcaa |
| | | tgctttatccttatctcaataatccttattaaacacagaggaggtaagaaatcatgactt |
| | | tatgttatgaatttgtaacttaaagttcacagtggcctaatttttgccatgcacaaaata |
| | | tcagcacaagaataacatgtccatccatccataaagaaacaataaattaaataatcatca |
| | | aaatcagatacaatcctagaccaccacgctagaaatatcttcttggccctatcataaaaa |
| | | ccgtgaaagtgagattgaagctcacggcacatttccatttataatcatggactatgtacc |
| | | taaacttttaaaacaggtgtgattttaaataaatcactgccaggttcctggaagacatgc |
| | | agaataagtggtcatcattatagcatcaatgaagtagcgaggaattcgtggcctggaatt |
| | | aggataggtggtatcaactcaaaactacacccagaaatgacctaacaaaagattgcaaag |
| | | atcattggtttctaaaaacctacaaagtatttaaatgagtgtagtaagtgaatatagact |
| | | tatttaaatctgtattaagcatattctaatatatatataatgtatatattatatatgtac |
| | | atatatatggtactaaaacattgcatagacttaaacataatctcattgttagaaatcaac |
| | | aaatatcattaaaagttaggattcactttagagcattaaaaaaccacaaaaccactaaat |
| | | aacatataatctaacaaacatacatagaatattttacaaggttgttataactcattaaac |
| | | tagcttaatctttaagcatctacactaaagattctttatgatatcagccttcaattatca |
| | | aggctgatggcttattattttctaaagataatcatcatagaaaattagtgaatatgtttt |
| | | atagaaatttcaggttataacatttagtacaacttttagaaactgaaaaacacaatcata |
| | | gggacataaaatatacatctgtaaaaaataatttttctatccttccatagaaaatgcaca |
| | | ctttgctgggcttacatatatttcaatgttttccattttttattatgagactattaacac |
| | | agagcaattatattttagaagactataattttctatatattttccacactgccaatccaa |
| | | gttagtccagtcttcagaataagaagtacacagaccggaacttaggaaattagtctgaac |
| | | aggtgagagggtgcgccagagaacctgacagcttctggaacaggcggaagcacagaggcg |
| | | ctgaggcatcaccctgtgtgggccggggacagccggccaccttctggaccagaggacagg |
| | | tgcccgcccggctggggaggcggcctaagccacagcagcagcggtcgccatcttggtccg |
| | | ggacccgccgaacttaggaaattagtctgaacaggtgagagggtgcgccagagaacctga |
| | | cagcttctggaacaggcagaagcacagaggcgctgaggcagcaccctgtgtgggccgggg |
| | | acagccggccaccttccggaccagaggacaggtgcccgcccggctggggaggcggcctaa |
| | | gccacagcagcagcggtcgccatcttggtccgggacccgccgaacttaggaaattagtct |
| | | gaacaggtgagagggtgcgccagagaacctgacagcttctggaacaggcggaagcacaga |
| | | ggcgctgaggcagcaccctttgtgggccggggacagccagccaccatccggaccggagga |
| | | caggtgcccgcccggctggggaggcggcctaagccacagcagcagcggtcgccatcttgg |
| | | tccgagacccgccgaacttaggaaattagtctgaacaggtgagagggtgcgccagagaac |
| | | ctgacagcttctagaacaggccgaagcacagaggcgctgaggcagcaccctgtgtgggcc |
| | | ggggacagccggccaccttctggaccagaggacaggtgcccgcccggctggggaggcggc |
| | | ctaagccacagcagcagcggtcgccatcttggtccgggacccgccgaacttaggaaatta |
| | | gtctgaacaggtgagagggtgcgccagagaacctgacagcttctggaacaggcagaagca |
| | | cagaggcgctgaggcagcaccctgtgtgggccggggacagccggccaccttccggaccag |
| | | aggacaggtgcccgcccggctggggaggcggcctaagccacagcagcagcggtcgccatc |
| | | ttggtccgggacccgccgaacttaggaaattagtctgaacaggtgagagggtgcgccaga |
| | | gaacctgacagcttctggaacaggcagaagcacagaggcgctgaggcagcaccctgtgtg |
| | | ggccggggacagccggccaccttccggaccagaggacaggtgcccgcccggctggggagg |
| | | cgacctaagccacagcagcagcggtcgccatcttggtccgggacccgccgaacttaggaa |
| | | attagtctgaacaggtgagagggtgcgccagagaacctgacagcttctggaacaggcgga |
| | | agcacagaggcgctgaggcagcaccctgtgtgggccggggacagccagccaccttcctga |
| | | ccggaggacaggtgcccacccggcaggggaggcggcctaagccacagcagcagcggtcgc |
| | | catcttggtcccgggactccaaggaacttaggaatttagtctgcttaggtgagagtctgt |
| | | accacctgggaactgccaaagcaacacagtgtctgagaaaggtcctgttttgggccttct |
| | | tcttcgtccaggaggaggtccaaatacaagatatctgcgcaccttccctgtaagagagct |
| | | tgccagcagagagtgctctgagcactgaaactcagaggagagaatctgtctcccaggtct |
| | | gctgatagacggtaacagaatcaccagaagaacaatctctaaacagagtcaactataact |
| | | actaactccagagattaccagatggcgaaaggtaaacggaggaatcttactaacaggaac |
| | | caagaccactcaccatcaccagaacccagcacacccacttcgcccagtccagggaacccc |
| | | aacacacctgagaacctagacctagatttaaaagcatatctcatgatgatggtagagggc |
| | | atcaagaaggactttaataaatcacttaaagaaatacaggagaacactgctaaagagtta |
| | | caagtccttaaagaaaaacaggaaaacacaatcaaacaggtagaagtccttacagaaaaa |
| | | gaggaaaaaacatacaaacaggtgatggaaatgaacaaaaccatactagacctaaaaagg |
| | | gaagtagacacaataaagaaaactcaaagcgaggcaacactagagatagaaaccctagga |
| | | aagaaatctggaaccatagatttgagcatcagcaacagaatacaagagatggaagagaga |
| | | atctcaggtgcagaacattccatagagaacatcggcacaacaatcaaagaaaatggaaaa |
| | | tgcaaaaagatcctaactcaaaatatccaggaaatccaggacacaataagaagaccaaac |
| | | gtacggataataggagtggatgagaatgaagattttcaactcaaaggtccagcaaacatc |
| | | ttcaacaaaattattgaagaaaacttcccaaatctaaagaatgagatgcatatgaacata |
| | | caagaagcctacagaactccaaatagactggaccagaaaagaaattcctcccgacacata |
| | | ataatcagaacatcaaatgcactaaataaagatagaatactaaaagcagtaagggaaaaa |
| | | ggtcaagtaacatataaaggcaagcctatcagaattacaccagatttttcaccagagact |
| | | atgaaagccagaagagcctggacagatgttatacagacactaagagaacacaaactgcag |
| | | cccaggctactatacccagccaaactctcaattatcatagagggagaaaccaaagtattc |
| | | cacgacaaaaccaaattcacgcattatctctccacgaatccagcccttcaaaggataata |
| | | acagaaaaaaaccaatacaagaacgggaacaacgccctagaaaaaacaagaaggtaatcc |
| | | ctcaacaaacctaaaagaagacagccacaagaacagaatgccacctttaacaactaaaat |
| | | aacaggaagcaacaattacttttccttaatatctcttaacatcaatggtctcaactcgcc |
| | | aataaaaagacatagactaacaaactggctacacaaacaagacccaacattttgctgctt |
| | | acaggaaactcatctcagagaaaaagatagacactacctcagaatgaaaggctggaaaac |
| | | aattttccaagcaaatggtatgaagaaacaagcaggagtagccatcctaatatctgataa |
| | | gattgacttccaacccaaagtcatcaaaaaagacaaggagggacacttcattctcatcaa |
| | | aggtaaaatcctccaagaggaactctcaattctgaatatctatgctccaaatacaagagc |
| | | agccacattcactaaagaaactttagtaaagctcaaagcacacattgcgcctcacacaat |
| | | aatagtgggagacttcaacacaccactttcaccaatggacagatcatggaaacagaaact |
| | | aaacagggacacactgaaactaacagaagtgatgaaacaaatggatctgacagatatcta |
| | | cagaacattttaccctaaaacaaaaggatataccttcttctcagcacctcatggtacctt |
| | | ctccaaaattgaccacataataggtcacaaatcaggcctcaacagattaaaaaatattga |
| | | aattgtcccatgtatcctatcagatcaccatgcactaaggctgatcttcaataacaaaat |
| | | aaataacagaaagccaacattcacatggaaactgaacaacactcttctcaatgatacctt |
| | | ggtcaaggaaggaataaagaaagaaattaaagactttttagagtttaatgaaaatgaagc |
| | | cacaacgtacccaaacctttgggacacaatgaaagcatttctaagagggaaactcatagc |
| | | tatgagtgccttcaagaaaaaacgggagagagcacatactagcagcttgacaacacatct |
| | | aaaagctctagaaaaaaaggaagcaaattcacccaagaggagtagacggcaggaaataat |
| | | caaactcaggggtgaaatcaaccaagtggaaacaagaagaactattcaaagaattaacca |
| | | aacgaggagttggttctttgagaaaatcaacaagatagataaacccttagctagactcac |
| | | taaagggcacagggacaaaatcctaattaacaaaatcagaaatgaaaagggagacataac |
| | | aacagatcctgaagaaatccaaaacaccatcagatccttctacaaaaggctatactcaac |
| | | aaaactggaaaacctggacgaaatggacaaatttctggacagataccaggtaccaaagtt |
| | | gaatcaggatcaagttgaccttctaaacagtcccatatcccctaaagaaatagaagcagt |
| | | tattaatagtctcccagccaaaaaaagcccaggaccagacgggtttagtgcagagttcta |
| | | tcagaccttcaaagaagatctaactccagttctgcacaaactttttcacaagatagaagt |
| | | agaaggtattctacccaactcattttatgaagccactattactctgatacctaaaccaca |
| | | gaaagatccaacaaagatagagaacttcagaccaatttctcttatgaacatcgatgcaaa |
| | | aatccttaataaaattctcgctaaccgaatccaagaacacattaaagcaatcatccatcc |
| | | tgaccaagtaggttttattccagggatgcagggatggtttaatatacgaaaatccatcaa |
| | | tgtaatccattatataaacaaactcaaagacaaaaaccacatgatcatctcgttagatgc |
| | | agaaaaagcatttgacaagatccaacacccattcatgataaaagttctggaaagatcagg |
| | | aattcaaggccaatacctaaacatgataaaagcaatctacagcaaaccagtagccaacat |
| | | caaagtaaatggagagaagctggaagcaatcccactaaaatcagggactagacaaggctg |
| | | cccactttctccctaccttttcaacatagtacttgaagtattagccagagcaattcgaca |
| | | acaaaaggagatcaaggggatacaaattggaaaagaggaagtcaaaatatcactttttgc |
| | | agatgatatgatagtatatataagtgaccctaaaaattccaacagagaactcctaaacct |
| | | gataaacagcttcggtgaagtagctggatataaaattaactcaaacaagtcaatggcctt |
| | | tctctacacaaagaataaacaggctgagaaagaaattagggaaacaacacccttctcaat |
| | | agccacaaataatataaaatatctcggcgtgactctaacgaaggaagtgaaagatctgta |
| | | tgataaaaacttcaagtccctgaagaaagaaattaaagaagatctcagaagatggaaaga |
| | | tctcccatgctcatggattggcaggaccaacattgtaaaaatggctatcttgccaaaagc |
| | | aatctacagattcaatgcaatccccattaaaattccaactcaattcttcaacgaattaga |
| | | aggagcaatttgcaaattcatctggaataacaaaaaaccgaggatagcaaaaactcttct |
| | | caaggataaaagaacctctggtggaatcaccatgcctgacctaaagctttactacagagc |
| | | aattgtgataaaaactgcatggtactggtatagagacagacaagtagaccaatggaatag |
| | | aattgaagacccagaaatgaacccacacacctatggtcacttgatcttcgacaagggagc |
| | | caaaaccatccagtggaagaaagacagcattttcaacaattggtgctggcacaactggtt |
| | | gttatcatgtagaagaatgcgaatcgatccatacttatctccttgtactaaggtcaaatc |
| | | taagtggatcaaggaacttcacataaaaccagagacactgaaacttatagaggagaaagt |
| | | ggggaaaagtcttgaagatatgggcacaggggaaaaattcctgaacagaacagcaatggc |
| | | ttgtgctgtaagatcgagaattgacaaatgggacctaatgaaactccaaagtttctgcaa |
| | | ggcaaaagacactgtctataagacaaaaagaccaccaacagactgggaaaggatotttac |
| | | ctatcctaaatcagataggggactaatatccaacatatataaagaactcaagaaggtgga |
| | | cctcagaaaatcaaataacccccttaaaaaatggggctcagaactgaacaaagaattctc |
| | | acctgaggaataccgaatggcagagaagcacctgaaaaaatgttcaacatccttaatcat |
| | | cagggaaatgcaaatcaaaacaaccctgagattccacctcacaccagtgagaatggctaa |
| | | gatcaaaaattcaggtgacagcagatgctggcgaggatgtggagaaagaggaacactcct |
| | | ccattgttggtgggattgcaggcttgtacaaccactctggaaatcagtctggcggttcct |
| | | cagaaaattggacatagtactaccggaggatccagcaatacctctcctgggcatatatcc |
| | | agaagaagccccaactggtaagaaggacacatgctccactatgttcatagcagccttatt |
| | | tataatagccagaaactggaaagaacccagatgcccctcaacagaggaatggatacagaa |
| | | aatgtggtacatctacacaatggagtactactcagctattaaaaagaatgaatttatgaa |
| | | attcctagccaaatggatggacctggagagcatcatcctgagtgaggtaacacaatcaca |
| | | aaggaactcacacaatatgtactcactgataagtggatactagcccaaaacctaggatac |
| | | ccacgatataagatacaatttcctaaacacatgaaactcaagaaaaatgaagactgaagt |
| | | gtggacactatgcccctccttagaagtgggaacaaaacacccatggaaggagttacagaa |
| | | acaaagtttggagctgagatgaaaggagggaccatgtagagactgccatatccagggatc |
| | | caccccataatcagcatccaaacgctgacaccattgcatatactagcaagattttatcga |
| | | aaggacccagatgtagctgtctcttgtgagactatgccggggcctagcaaacacagaagt |
| | | ggatgctcacagtcagctaatggatggatcacagggctcccaatggaggagctagagaaa |
| | | gtacccaaggagctaaagggatctgcaaccctataggtggaacaacattatgaactaacc |
| | | agtacccctgagctcttgactctagctgcatatgtatcaaaagatggcctagtcggccat |
| | | cactggaaagagaggcccattggacacgcagactttgtgtgccccggtacaggggaacgc |
| | | cagggccaaaggggggggagtgggtgggtaggggagtgggggtgggtgggtaagggggac |
| | | ttttggtatagcattggaaatgtaaatgagctaaatacctaataaaaaatggaaaaaaaa |
| | | taaaaaaaataaaataaaaaaaaagaccatgaaacataaaaaaaaaaaaaaaaaaaaaga |
| | | agtacacagactaacatcaactcgctaaaaagccaactacaatggaaagctctgtcttct |
| | | tcaagattagcctatgcagtatgatgttagtatgttcaaataagcataatggtttttcac |
| | | aataatagaatacattatctagatataaacattttattccccaaatcatttataacctca |
| | | tttacatacagctctcaagttgcattttgcagcgttgtgtgtccatgggaaacagagtta |
| | | agtccagaggacatgaaagtgtaatagacaacctgaaggcaagaacagaaaaattggaat |
| | | cattgagcgtacatcgctgagatccactgacttgaaaaggaaactacaaataaacaagat |
| | | ctaagtagagcagggtacctcatgctcacaaggacgtc |
| 95 | ΔL1spa | gtaaagcaattttttccaactgaacaggatctcctgactgccagatgaatcttaaatcat |
| | | ctttatagtattttgtacagttgccatattcgatatcctcctttttaatatcaaattgag |
| | | cggttgtatatccaactacaataagtaaataaacaagttagaggaaaatatgcatatcaa |
| | | tgctttatccttatctcaataatccttattaaacacagaggaggtaagaaatcatgactt |
| | | tatgttatgaatttgtaacttaaagttcacagtggcctaatttttgccatgcacaaaata |
| | | tcagcacaagaataacatgtccatccatccataaagaaacaataaattaaataatcatca |
| | | aaatcagatacaatcctagaccaccacgctagaaatatcttcttggccctatcataaaaa |
| | | ccgtgaaagtgagattgaagctcacggcacatttccatttataatcatggactatgtacc |
| | | taaacttttaaaacaggtgtgattttaaataaatcactgccaggttcctggaagacatgc |
| | | agaataagtggtcatcattatagcatcaatgaagtagcgaggaattcgtggcctggaatt |
| | | aggataggtggtatcaactcaaaactacacccagaaatgacctaacaaaagattgcaaag |
| | | atcattggtttctaaaaacctacaaagtatttaaatgagtgtagtaagtgaatatagact |
| | | tatttaaatctgtattaagcatattctaatatatatataatgtatatattatatatgtac |
| | | atatatatggtactaaaacattgcatagacttaaacataatctcattgttagaaatcaac |
| | | aaatatcattaaaagttaggattcactttagagcattaaaaaaccacaaaaccactaaat |
| | | aacatataatctaacaaacatacatagaatattttacaaggttgttataactcattaaac |
| | | tagcttaatctttaagcatctacactaaagattctttatgatatcagccttcaattatca |
| | | aggctgatggcttattattttctaaagataatcatcatagaaaattagtgaatatgtttt |
| | | atagaaatttcaggttataacatttagtacaacttttagaaactgaaaaacacaatcata |
| | | gggacataaaatatacatctgtaaaaaataatttttctatccttccatagaaaatgcaca |
| | | ctttgctgggcttacatatatttcaatgttttccattttttattatgagactattaacac |
| | | agagcaattatattttagaagactataattttctatatattttccacactgccaatccaa |
| | | gttagtccagtcttcagaataagaagtacacagaccggaacttaggaaattagtctgaac |
| | | aggtgagagggtgcgccagagaacctgacagcttctggaacaggcggaagcacagaggcg |
| | | ctgaggcatcaccctgtgtgggccggggacagccggccaccttctggaccagaggacagg |
| | | tgcccgcccggctggggaggcggcctaagccacagcagcagcggtcgccatcttggtccg |
| | | ggacccgccgaacttaggaaattagtctgaacaggtgagagggtgcgccagagaacctga |
| | | cagcttctggaacaggcagaagcacagaggcgctgaggcagcaccctgtgtgggccgggg |
| | | acagccggccaccttccggaccagaggacaggtgcccgcccggctggggaggcggcctaa |
| | | gccacagcagcagcggtcgccatcttggtccgggacccgccgaacttaggaaattagtct |
| | | gaacaggtgagagggtgcgccagagaacctgacagcttctggaacaggcggaagcacaga |
| | | ggcgctgaggcagcaccctttgtgggccggggacagccagccaccatccggaccggagga |
| | | caggtgcccgcccggctggggaggcggcctaagccacagcagcagcggtcgccatcttgg |
| | | tccgagacccgccgaacttaggaaattagtctgaacaggtgagagggtgcgccagagaac |
| | | ctgacagcttctagaacaggccgaagcacagaggcgctgaggcagcaccctgtgtgggcc |
| | | ggggacagccggccaccttctggaccagaggacaggtgcccgcccggctggggaggcggc |
| | | ctaagccacagcagcagcggtcgccatcttggtccgggacccgccgaacttaggaaatta |
| | | gtctgaacaggtgagagggtgcgccagagaacctgacagcttctggaacaggcagaagca |
| | | cagaggcgctgaggcagcaccctgtgtgggccggggacagccggccaccttccggaccag |
| | | aggacaggtgcccgcccggctggggaggcggcctaagccacagcagcagcggtcgccatc |
| | | ttggtccgggacccgccgaacttaggaaattagtctgaacaggtgagagggtgcgccaga |
| | | gaacctgacagcttctggaacaggcagaagcacagaggcgctgaggcagcaccctgtgtg |
| | | ggccggggacagccggccaccttccggaccagaggacaggtgcccgcccggctggggagg |
| | | cgacctaagccacagcagcagcggtcgccatcttggtccgggacccgccgaacttaggaa |
| | | attagtctgaacaggtgagagggtgcgccagagaacctgacagcttctggaacaggcgga |
| | | agcacagaggcgctgaggcagcaccctgtgtgggccggggacagccagccaccttcctga |
| | | ccggaggacaggtgcccacccggcaggggaggcggcctaagccacagcagcagcggtcgc |
| | | catcttggtcccgggactccaaggaacttaggaatttagtctgcttaggtgagagtctgt |
| | | accacctgggaactgccaaagcaacacagtgtctgagaaaggtcctgttttgggccttct |
| | | tcttcgtccaggaggaggtccaaatacaagatatctgcgcaccttccctgtaagagagct |
| | | tgccagcagagagtgctctgagcactgaaactcagaggagagaatctgtctcccaggtct |
| | | gctgatagacggtaacagaatcaccagaagaacaatctctaaacagagtcaactataact |
| | | actaactccagagattaccagatggcgaaaggtaaacggaggaatcttactaacaggaac |
| | | caagaccactcaccatcaccagaacccagcacacccacttcgcccagtccagggaacccc |
| | | aacacacctgagaacctagacctagatttaaaagcatatctcatgatgatggtagagggc |
| | | atcaagaaggactttaataaatcacttaaagaaatacaggagaacactgctaaagagtta |
| | | caagtccttaaagaaaaacaggaaaacacaatcaaacaggtagaagtccttacagaaaaa |
| | | gaggaaaaaacatacaaacaggtgatggaaatgaacaaaaccatactagacctaaaaagg |
| | | gaagtagacacaataaagaaaactcaaagcgaggcaacactagagatagaaaccctagga |
| | | aagaaatctggaaccatagatttgagcatcagcaacagaatacaagagatggaagagaga |
| | | atctcaggtgcagaacattccatagagaacatcggcacaacaatcaaagaaaatggaaaa |
| | | tgcaaaaagatcctaactcaaaatatccaggaaatccaggacacaataagaagaccaaac |
| | | gtacggataataggagtggatgagaatgaagattttcaactcaaaggtccagcaaacatc |
| | | ttcaacaaaattattgaagaaaacttcccaaatctaaagaatgagatgcatatgaacata |
| | | caagaagcctacagaactccaaatagactggaccagaaaagaaattcctcccgacacata |
| | | ataatcagaacatcaaatgcactaaataaagatagaatactaaaagcagtaagggaaaaa |
| | | ggtcaagtaacatataaaggcaagcctatcagaattacaccagatttttcaccagagact |
| | | g |
| | | cccaggctactatacccagccaaactctcaattatcatagagggagaaaccaaagtattc |
| | | cacgacaaaaccaaattcacgcattatctctccacgaatccagcccttcaaaggataata |
| | | acagaaaaaaaccaatacaagaacgggaacaacgccctagaaaaaacaagaaggtaatcc |
| | | ctcaacaaacctaaaagaagacagccacaagaacagaatgccacctttaacaactaaaat |
| | | aacaggaagcaacaattacttttccttaatatctcttaacatcaatggtctcaactcgcc |
| | | aataaaaagacatagactaacaaactggctacacaaacaagacccaacattttgctgctt |
| | | acaggaaactcatctcagagaaaaagatagacactacctcagaatgaaaggctggaaaac |
| | | aattttccaagcaaatggtatgaagaaacaagcaggagtagccatcctaatatctgataa |
| | | gattgacttccaacccaaagtcatcaaaaaagacaaggagggacacttcattctcatcaa |
| | | aggtaaaatcctccaagaggaactctcaattctgaatatctatgctccaaatacaagagc |
| | | agccacattcactaaagaaactttagtaaagctcaaagcacacattgcgcctcacacaat |
| | | aatagtgggagacttcaacacaccactttcaccaatggacagatcatggaaacagaaact |
| | | aaacagggacacactgaaactaacagaagtgatgaaacaaatggatctgacagatatcta |
| | | cagaacattttaccctaaaacaaaaggatataccttcttctcagcacctcatggtacctt |
| | | ctccaaaattgaccacataataggtcacaaatcaggcctcaacagattaaaaaatattga |
| | | aattgtcccatgtatcctatcagatcaccatgcactaaggctgatcttcaataacaaaat |
| | | aaataacagaaagccaacattcacatggaaactgaacaacactcttctcaatgatacctt |
| | | ggtcaaggaaggaataaagaaagaaattaaagactttttagagtttaatgaaaatgaagc |
| | | cacaacgtacccaaacctttgggacacaatgaaagcatttctaagagggaaactcatagc |
| | | tatgagtgccttcaagaaaaaacgggagagagcacatactagcagcttgacaacacatct |
| | | aaaagctctagaaaaaaaggaagcaaattcacccaagaggagtagacggcaggaaataat |
| | | caaactcaggggtgaaatcaaccaagtggaaacaagaagaactattcaaagaattaacca |
| | | aacgaggagttggttctttgagaaaatcaacaagatagataaacccttagctagactcac |
| | | taaagggcacagggacaaaatcctaattaacaaaatcagaaatgaaaagggagacataac |
| | | aacagatcctgaagaaatccaaaacaccatcagatccttctacaaaaggctatactcaac |
| | | aaaactggaaaacctggacgaaatggacaaatttctggacagataccaggtaccaaagtt |
| | | gaatcaggatcaagttgaccttctaaacagtcccatatcccctaaagaaatagaagcagt |
| | | tattaatagtctcccagccaaaaaaagcccaggaccagacgggtttagtgcagagttcta |
| | | tcagaccttcaaagaagatctaactccagttctgcacaaactttttcacaagatagaagt |
| | | agaaggtattctacccaactcattttatgaagccactattactctgatacctaaaccaca |
| | | gaaagatccaacaaagatagagaacttcagaccaatttctcttatgaacatcgatgcaaa |
| | | aatccttaataaaattctcgctaaccgaatccaagaacacattaaagcaatcatccatcc |
| | | tgaccaagtaggttttattccagggatgcagggatggtttaatatacgaaaatccatcaa |
| | | tgtaatccattatataaacaaactcaaagacaaaaaccacatgatcatctcgttagatgc |
| | | agaaaaagcatttgacaagatccaacacccattcatgataaaagttctggaaagatcagg |
| | | aattcaaggccaatacctaaacatgataaaagcaatctacagcaaaccagtagccaacat |
| | | caaagtaaatggagagaagctggaagcaatcccactaaaatcagggactagacaaggctg |
| | | cccactttctccctaccttttcaacatagtacttgaagtattagccagagcaattcgaca |
| | | acaaaaggagatcaaggggatacaaattggaaaagaggaagtcaaaatatcactttttgc |
| | | agatgatatgatagtatatataagtgaccctaaaaattccaacagagaactcctaaacct |
| | | gataaacagcttcggtgaagtagctggatataaaattaactcaaacaagtcaatggcctt |
| | | tctctacacaaagaataaacaggctgagaaagaaattagggaaacaacacccttctcaat |
| | | agccacaaataatataaaatatctcggcgtgactctaacgaaggaagtgaaagatctgta |
| | | tgataaaaacttcaagtccctgaagaaagaaattaaagaagatctcagaagatggaaaga |
| | | tctcccatgctcatggattggcaggaccaacattgtaaaaatggctatcttgccaaaagc |
| | | aatctacagattcaatgcaatccccattaaaattccaactcaattcttcaacgaattaga |
| | | aggagcaatttgcaaattcatctggaataacaaaaaaccgaggatagcaaaaactcttct |
| | | caaggataaaagaacctctggtggaatcaccatgcctgacctaaagctttactacagagc |
| | | aattgtgataaaaactgcatggtactggtatagagacagacaagtagaccaatggaatag |
| | | aattgaagacccagaaatgaacccacacacctatggtcacttgatcttcgacaagggagc |
| | | caaaaccatccagtggaagaaagacagcattttcaacaattggtgctggcacaactggtt |
| | | gttatcatgtagaagaatgcgaatcgatccatacttatctccttgtactaaggtcaaatc |
| | | taagtggatcaaggaacttcacataaaaccagagacactgaaacttatagaggagaaagt |
| | | ggggaaaagtcttgaagatatgggcacaggggaaaaattcctgaacagaacagcaatggc |
| | | ttgtgctgtaagatcgagaattgacaaatgggacctaatgaaactccaaagtttctgcaa |
| | | ggcaaaagacactgtctataagacaaaaagaccaccaacagactgggaaaggatctttac |
| | | ctatcctaaatcagataggggactaatatccaacatatataaagaactcaagaaggtgga |
| | | cctcagaaaatcaaataacccccttaaaaaatggggctcagaactgaacaaagaattctc |
| | | acctgaggaataccgaatggcagagaagcacctgaaaaaatgttcaacatccttaatcat |
| | | cagggaaatgcaaatcaaaacaaccctgagattccacctcacaccagtgagaatggctaa |
| | | gatcaaaaattcaggtgacagcagatgctggcgaggatgtggagaaagaggaacactcct |
| | | ccattgttggtgggattgcaggcttgtacaaccactctggaaatcagtctggcggttcct |
| | | cagaaaattggacatagtactaccggaggatccagcaatacctctcctgggcatatatcc |
| | | agaagaagccccaactggtaagaaggacacatgctccactatgttcatagcagccttatt |
| | | tataatagccagaaactggaaagaacccagatgcccctcaacagaggaatggatacagaa |
| | | aatgtggtacatctacacaatggagtactactcagctattaaaaagaatgaatttatgaa |
| | | attcctagccaaatggatggacctggagagcatcatcctgagtgaggtaacacaatcaca |
| | | aaggaactcacacaatatgtactcactgataagtggatactagcccaaaacctaggatac |
| | | ccacgatataagatacaatttcctaaacacatgaaactcaagaaaaatgaagactgaagt |
| | | gtggacactatgcccctccttagaagtgggaacaaaacacccatggaaggagttacagaa |
| | | acaaagtttggagctgagatgaaaggagggaccatgtagagactgccatatccagggatc |
| | | caccccataatcagcatccaaacgctgacaccattgcatatactagcaagattttatcga |
| | | aaggacccagatgtagctgtctcttgtgagactatgccggggcctagcaaacacagaagt |
| | | ggatgctcacagtcagctaatggatggatcacagggctcccaatggaggagctagagaaa |
| | | gtacccaaggagctaaagggatctgcaaccctataggtggaacaacattatgaactaacc |
| | | agtacccctgagctcttgactctagctgcatatgtatcaaaagatggcctagtcggccat |
| | | cactggaaagagaggcccattggacacgcagactttgtgtgccccggtacaggggaacgc |
| | | cagggccaaaggggggggagtgggtgggtaggggagtgggggtgggtgggtaagggggac |
| | | ttttggtatagcattggaaatgtaaatgagctaaatacctaataaaaaatggaaaaaaaa |
| | | taaaaaaaataaaataaaaaaaaagaccatgaaacataaaaaaaaaaaaaaaaaaaaaga |
| | | agtacacagactaacatcaactcgctaaaaagccaactacaatggaaagctctgtcttct |
| | | tcaagattagcctatgcagtatgatgttagtatgttcaaataagcataatggtttttcac |
| | | aataatagaatacattatctagatataaacattttattccccaaatcatttataacctca |
| | | tttacatacagctctcaagttgcattttgcagcgttgtgtgtccatgggaaacagagtta |
| | | agtccagaggacatgaaagtgtaatagacaacctgaaggcaagaacagaaaaattggaat |
| | | cattgagcgtacatcgctgagatccactgacttgaaaaggaaactacaaataaacaagat |
| | | ctaagtagagcagggtacctcatgctcacaaggacgtc |

### Example 7—In Vivo L1 RNA Depletion Ameliorated the Senescent Phenotype and Increased the Lifespan of LAKI Mice

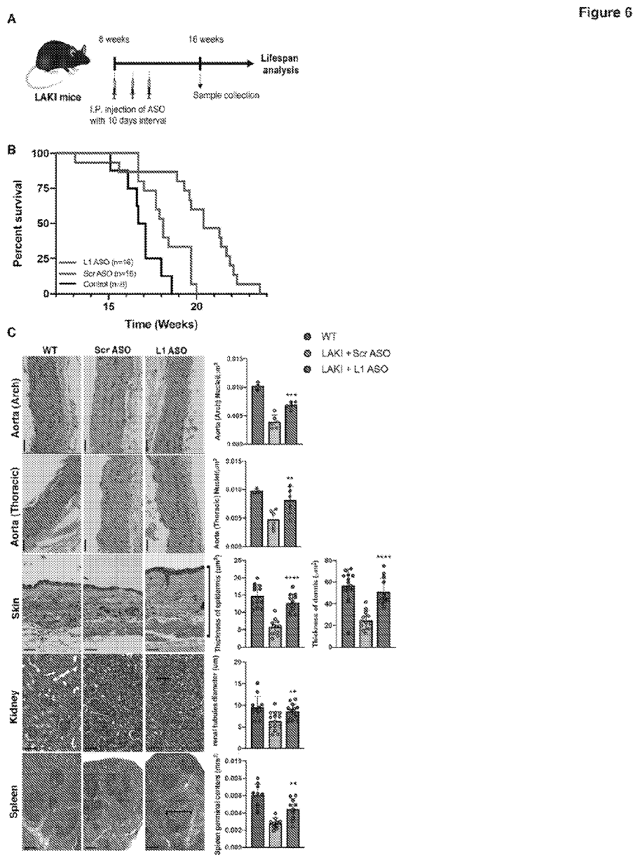

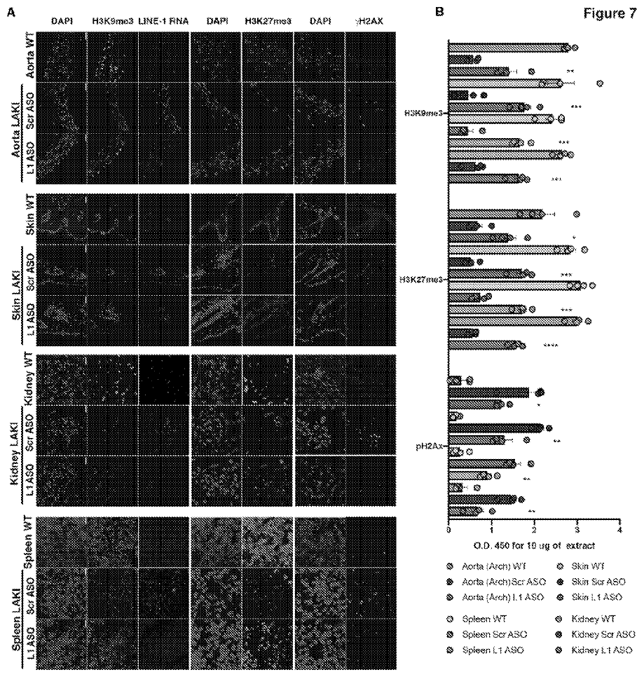

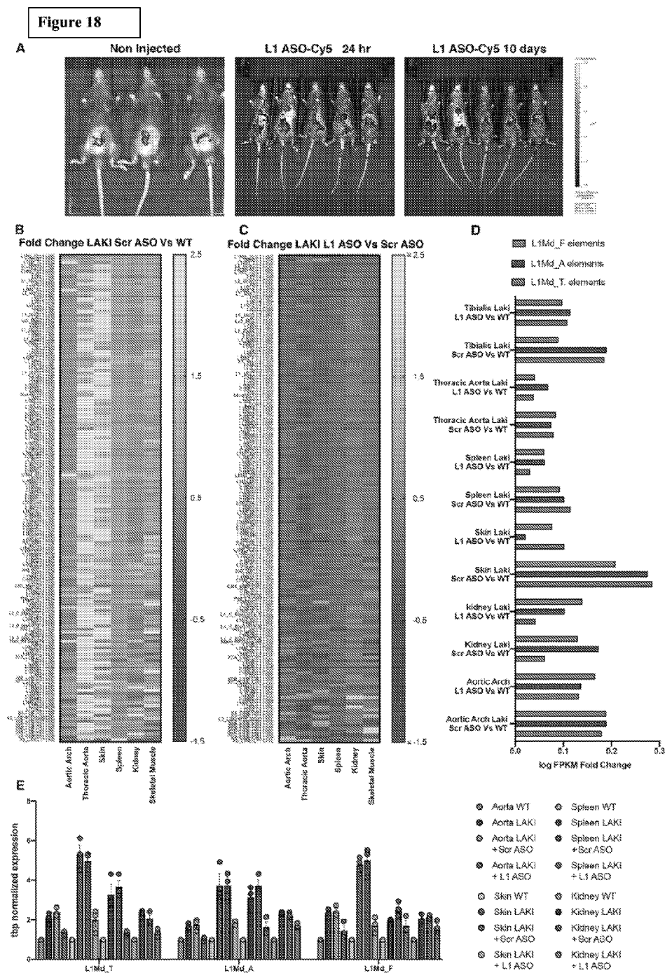

To test whether L1 RNA depletion in vivo could have any beneficial effect on LAKI mice in preventing the onset of the senescence phenotype, we treated LAKI mice with 2′F-ANA ASO starting at 8-weeks of age. Mice were treated with 3 rounds of intraperitoneal injection of either L1 ASO (2 mg/kg) or Scr ASO with a 10 days interval between each injection (FIG. 6A). We analyzed the in vivo distribution and stability of the ASO by injecting Cy5-labeled L1 ASO and monitoring the fluorophore signal emission via an in vivo imaging system (IVIS) (FIG. 18A). Using RNA-seq, we analyzed L1 expression in different organs of LAKI mice. Compared to WT mice, many subfamilies of L1 elements were deregulated in the aortic arch, thoracic aorta, skin, spleen, kidney, and skeletal muscle (tibialis anterior), and L1 ASO injection reduced the global expression of L1 RNA in all analyzed tissues, particularly L1-Tf, L1-Gf, and L1-Af subfamilies (FIG. 18B). We also confirmed the L1 knockdown efficiency in the tissues of L1 ASO-injected LAKI mice by RNA-seq and qPCR (FIG. 18, C to E). L1 ASO treatment increased the median lifespan of LAKI mice compared to Scr ASO-injected mice (FIG. 6B). LAKI mice were sacrificed at 16 weeks of age, after 8-weeks of Scr ASO and L1 ASO injection, for molecular and histological analyses of the most affected tissues in LAKI mice, which were the aorta (both aortic arch and thoracic aorta), skin, spleen, and kidney (6, 43-45). Hematoxylin and eosin (H&E) staining revealed that mice injected with L1 ASO had an improved histological profile of the aorta (nuclei density per um2), skin (epidermal and dermal thickness), spleen (dimension of the germinal center area), and kidney (glomerulosclerosis) (FIG. 6C). L1 ASO treatment also restored H3K9me3 and H3K27me3 heterochromatin marks and reduced pH2Ax when compared to Scr ASO injected mice (FIGS. 7, A and B).

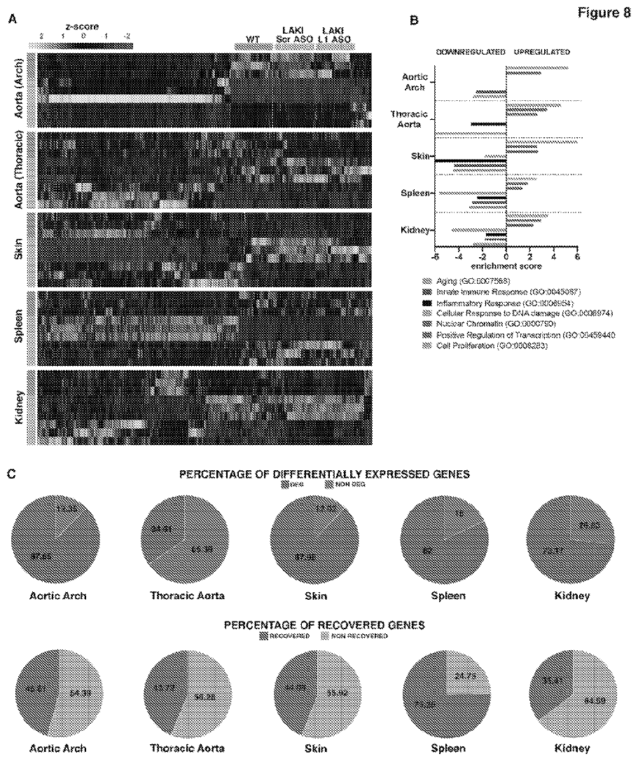

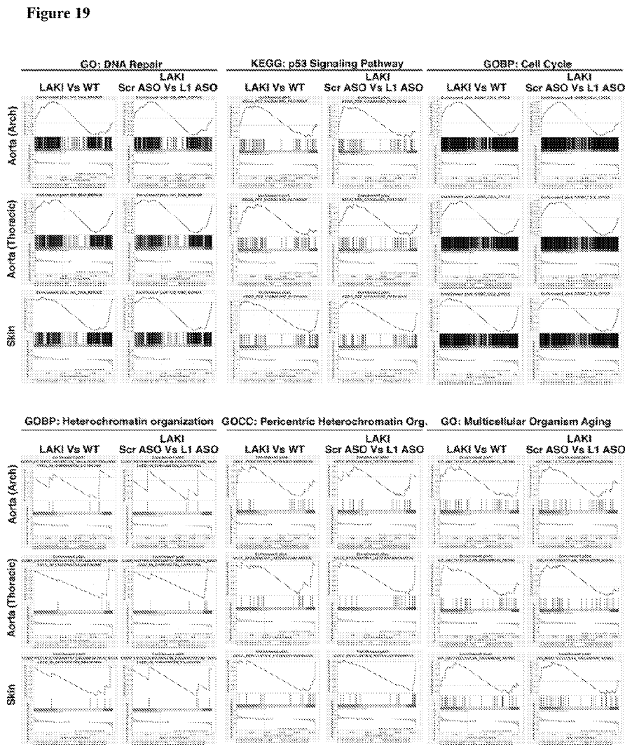

RNA-seq analysis was performed on the tissues from WT, Scr ASO, and L1 ASO-injected LAKI mice following histological analyses. Consistent with the histology data, differentially expressed genes analysis showed the recovery of the gene expression profile upon L1 ASO injection in LAKI mice (FIG. 8A). In addition, we performed a gene ontology analysis which revealed that in L1 ASO-treated mice, the pathways associated with aging, inflammatory response, innate immune response, and DNA damage were downregulated, whereas nuclear chromatin organization, cell proliferation, and transcription regulation pathways were enriched (FIGS. 8, B and C). The gene set enrichment analysis revealed the downregulation of the genes associated with multicellular organism aging, p53 senescence-associated pathway, and upregulation of pathways involved in the DNA repair, the cell cycle and the heterochromatin 3D Organization (FIG. 19). Together, these results confirmed that a stable reduction of L1 RNA restored H3K9me3, improved age-associated histological changes in multiple organs, restored gene expression, and increased the lifespan of LAKI mice.

### Example 8—Discussion

Endogenous L1 elements are transcriptionally active in both pathological (progeroid syndromes, FIG. 1, A to C) and physiologically aged cells (1, 2, 11, 12) (FIG. 20 (46)). In this study, we showed that L1 RNA accumulation is a common phenomenon observed among the progeroid syndromes, including the ones without a known mutation. Moreover, using HGPS and WRN patient cells, we showed that the accumulation of L1 RNA in the nucleus is an early event and precedes the loss of heterochromatin and increased expression of senescence-associated genes. We further demonstrated that the knockdown of L1 repetitive RNA using ASOs prevented heterochromatin de-condensation by releasing SUV39H activity and preserving H3K9me3 and H3K27me3 histone marks. This is associated with a reduction in DNA methylation age and in the expression of age-associated genes. Furthermore, L1 RNA depletion in vivo in LAKI mice delayed the onset of premature aging phenotypes in different tissues and increased the lifespan of treated mice. We demonstrated a previously undescribed function of L1 RNA as a negative regulator of SUV39H1 enzymatic activity, which might help to explain previous studies on L1 expression and its effects on chromatin opening and activation in early embryo development (13, 14). L1 ASO treatment, in addition to preventing the detrimental accumulation of L1 RNA inside the nucleus, also prevented the reverse transcription of proinflammatory L1 cDNA molecules in the cytoplasm (11, 12, 47) and its potential retrotransposition into the genome (10, 48) without the side effects typically observed with nucleotide reverse transcriptase inhibitor (NRTI) antiretroviral compounds like 3TC, which have recently been proposed as anti-aging compounds (49-51).

Prior to the invention, the therapeutic options for pathological premature aging syndromes were few and limited to protein prenylation inhibitors (farnesyltransferase inhibitors, FTIs) (41). However, FTIs are helpful only for HGPS patients (with a mutation in LMNA G609G) and are not an option for other progeroid syndromes caused by different mutations not associated with progerin accumulation. Studies using FTIs have revealed that intracellular accumulation of nonfarnesylated protein can cause hepatocellular diseases and cardiomyopathies (52, 53). Moreover, a recent clinical trial conducted on 25 patients with HGPS raised concerns regarding FTI efficacy (54). ASOs have been recently used to perform the skipping of the C1824T (G609G) mutation in HGPS syndrome (42, 55). However, for mutations in relevant protein domains (for example, the LMNA R644C mutation in the Zmpste24 cleavage site), the mutation skipping would lead to alterations in the reading frame and protein structure and function. Further, mutation skipping is not applicable for deletions (Werner syndromes) or progeroid syndromes caused by unknown mutations. Recent reports also demonstrated that correction of the C1824T mutation in LMNA (responsible for progeria syndromes) or deletion of Lmna transcripts using CRISPR-Cas9 genome editing tools were able to rescue the pathological phenotype in progeria mouse models (56-59).

In summary, it was demonstrated that an ASO-based approach against L1 repetitive RNA can ameliorate aging hallmarks in cells derived from patients with premature aging progeroid syndromes, including ones without a known mutation. L1 antisense oligonucleotides also ameliorated aging hallmarks and increased lifespans in LAKI mice without altering the genome or the post-translational modifications of the proteome (such as farnesylation). This study unveils a new avenue for the treatment of premature aging.

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

### REFERENCES

1. M. De Cecco, S. W. Criscione, E. J. Peckham, S. Hillenmeyer, E. A. Hamm, J. Manivannan, A. L. Peterson, J. A. Kreiling, N. Neretti, J. M. Sedivy, Genomes of replicatively senescent cells undergo global epigenetic changes leading to gene silencing and activation of transposable elements., Aging Cell 12, 247-56 (2013).
2. M. Van Meter, M. Kashyap, S. Rezazadeh, A. J. Geneva, T. D. Morello, A. Seluanov, V. Gorbunova, SIRT6 represses LINE1 retrotransposons by ribosylating KAP1 but this repression fails with stress and age, Nat. Commun. 5, 5011 (2014).
3. F. Feuerbach, V. Galy, E. Trelles-Sticken, M. Fromont-Racine, A. Jacquier, E. Gilson, J. C. Olivo-Marin, H. Scherthan, U. Nehrbass, Nuclear architecture and spatial positioning help establish transcriptional states of telomeres in yeast, Nat. Cell Biol. 4, 214-221 (2002).
4. L. Guelen, L. Pagie, E. Brasset, W. Meuleman, M. B. Faza, W. Talhout, B. H. Eussen, A. De Klein, L. Wessels, W. De Laat, B. Van Steensel, Domain organization of human chromosomes revealed by mapping of nuclear lamina interactions, Nature 453, 948-951 (2008).
5. P. Scaffidi, T. Misteli, Reversal of the cellular phenotype in the premature aging disease Hutchinson-Gilford progeria syndrome, Nat. Med. 11, 440-445 (2005).
6. F. G. Osorio, C. L. Navarro, J. Cadinanos, I. C. López-Mejia, P. M. Quirós, C. Bartoli, J. Rivera, J. Tazi, G. Guzmin, I. Varela, D. Depetris, F. de Carlos, J. Cobo, V. Andres, A. De Sandre-Giovannoli, J. M. P. Freije, N. Levy, C. Löpez-Otin, Splicing-directed therapy in a new mouse model of human accelerated aging., Sci. Transl. Med. 3, 106ra107 (2011).
7. A. Ocampo, P. Reddy, P. Martinez-Redondo, A. Platero-Luengo, F. Hatanaka, T. Hishida, M. Li, D. Lam, M. Kurita, E. Beyret, T. Araoka, E. Vazquez-Ferrer, D. Donoso, J. L. Roman, J. Xu, C. Rodriguez Esteban, G. Nunez, E. Nunez Delicado, J. M. Campistol, I. Guillen, P. Guillen, J. C. Izpisua Belmonte, In Vivo Amelioration of Age-Associated Hallmarks by Partial Reprogramming, Cell 167, 1719-1733.e12 (2016).
8. W. Zhang, J. Li, K. Suzuki, J. Qu, P. Wang, J. Zhou, X. Liu, R. Ren, X. Xu, A. Ocampo, T. Yuan, J. Yang, Y. Li, L. Shi, D. Guan, H. Pan, S. Duan, Z. Ding, M. Li, F. Yi, R. Bai, Y. Wang, C. Chen, F. Yang, X. Li, Z. Wang, E. Aizawa, A. Goebl, R. D. Soligalla, P. Reddy, C. R. Esteban, F. Tang, G.-H. Liu, J. C. I. Belmonte, Aging stem cells. A Werner syndrome stem cell model unveils heterochromatin alterations as a driver of human aging., Science 348, 1160-3 (2015).
9. J. G. Wood, S. L. Helfand, Chromatin structure and transposable elements in organismal aging., Front. Genet. 4, 274 (2013).
10. A. Sturm, Z. Ivics, T. Vellai, The mechanism of ageing: primary role of transposable elements in genome disintegration., Cell. Mol. Life Sci. 72, 1839-47 (2015).
11. M. De Cecco, T. Ito, A. P. Petrashen, A. E. Elias, N. J. Skvir, S. W. Criscione, A. Caligiana, G. Brocculi, E. M. Adney, J. D. Boeke, O. Le, C. Beausejour, J. Ambati, K. Ambati, M. Simon, A. Seluanov, V. Gorbunova, P. E. Slagboom, S. L. Helfand, N. Neretti, J. M. Sedivy, L1 drives IFN in senescent cells and promotes age-associated inflammation, Nature 566, 73-78 (2019).
12. M. Simon, M. Van Meter, J. Ablaeva, Z. Ke, R. S. Gonzalez, T. Taguchi, M. De Cecco, K. I. Leonova, V. Kogan, S. L. Helfand, N. Neretti, A. Roichman, H. Y. Cohen, M. V. Meer, V. N. Gladyshev, M. P. Antoch, A. V. Gudkov, J. M. Sedivy, A. Seluanov, V. Gorbunova, LINE1 Derepression in Aged Wild-Type and SIRT6-Deficient Mice Drives Inflammation, Cell Metab. 29, 871-885.e5 (2019).
13. J. W. Jachowicz, X. Bing, J. Pontabry, A. Boskovid, O. J. Rando, M. E. Torres-Padilla, LINE-1 activation after fertilization regulates global chromatin accessibility in the early mouse embryo, Nat. Genet. 49, 1502-1510 (2017).
14. M. Percharde, C.-J. Lin, Y. Yin, J. Guan, G. A. Peixoto, A. Bulut-Karslioglu, S. Biechele, B. Huang, X. Shen, M. Ramalho-Santos, A LINE1-Nucleolin Partnership Regulates Early Development and ESC Identity, Cell 174, 391-405.e19 (2018).
15. J. G. Wood, B. C. Jones, N. Jiang, C. Chang, S. Hosier, P. Wickremesinghe, M. Garcia, D. A. Hartnett, L. Burhenn, N. Neretti, S. L. Helfand, Chromatin-modifying genetic interventions suppress age-associated transposable element activation and extend life span in Drosophila, Proc. Natl. Acad. Sci. 113, 11277-11282 (2016).
16. W. R. Yang, D. Ardeljan, C. N. Pacyna, L. M. Payer, K. H. Burns, SQuIRE reveals locus-specific regulation of interspersed repeat expression, Nucleic Acids Res. 47, e27-e27 (2019).
17. A. Garg, L. Subramanyam, A. K. Agarwal, V. Simha, B. Levine, M. R. D′Apice, G. Novelli, Y. Crow, Atypical Progeroid Syndrome due to Heterozygous Missense LMNA Mutations, J. Clin. Endocrinol. Metab. 94, 4971-4983 (2009).
18. A. B. Csoka, H. Cao, P. J. Sammak, D. Constantinescu, G. P. Schatten, R. A. Hegele, Novel lamin A/C gene (LMNA) mutations in atypical progeroid syndromes., J. Med. Genet. 41, 304-8 (2004).
19. H. Cao, R. A. Hegele, LMNA is mutated in Hutchinson-Gilford progeria (MIM 176670) but not in Wiedemann-Rautenstrauch progeroid syndrome (MIM 264090)., J. Hum. Genet. 48, 271-274 (2003).
20. D. Ardeljan, X. Wang, M. Oghbaie, M. S. Taylor, D. Husband, V. Deshpande, J. P. Steranka, M. Gorbounov, W. R. Yang, B. Sie, H. B. Larman, H. Jiang, K. R. Molloy, I. Altukhov, Z. Li, W. McKerrow, D. Fenyo, K. H. Burns, J. Lacava, LINE-1 ORF2p expression is nearly imperceptible in human cancers, Mob. DNA (2019), doi:10.1186/s13100-019-0191-2.
21. P. H. Maxwell, What might retrotransposons teach us about aging?, Curr. Genet. 62, 277-82 (2016).
22. D. Andrenacci, V. Cavaliere, G. Lattanzi, The role of transposable elements activity in aging and their possible involvement in laminopathic diseases., Ageing Res. Rev. 57, 100995 (2020).
23. M. M. Karimi, P. Goyal, I. A. Maksakova, M. Bilenky, D. Leung, J. X. Tang, Y. Shinkai, D. L. Mager, S. Jones, M. Hirst, M. C. Lorincz, DNA methylation and SETDB1/H3K9me3 regulate predominantly distinct sets of genes, retroelements, and chimeric transcripts in mescs, Cell Stem Cell (2011), doi:10.1016/j.stem.2011.04.004.
24. A. Bulut-Karslioglu, I. A. DeLaRosa-Velázquez, F. Ramirez, M. Barenboim, M. Onishi-Seebacher, J. Arand, C. Galin, G. E. Winter, B. Engist, B. Gerle, R. J. O'Sullivan, J. H. A. Martens, J. Walter, T. Manke, M. Lachner, T. Jenuwein, Suv39h-Dependent H3K9me3 Marks Intact Retrotransposons and Silences LINE Elements in Mouse Embryonic Stem Cells, Mol. Cell (2014), doi:10.1016/j.molcel.2014.05.029.
25. K. H. C. Wei, C. Chan, D. Bachtrog, Establishment of h3k9me3-dependent heterochromatin during embryogenesis in drosophila miranda, Elife (2021), doi:10.7554/eLife.55612.
26. B. N. Vazquez, J. K. Thackray, N. G. Simonet, S. Chahar, N. Kane-Goldsmith, S. J. Newkirk, S. Lee, J. Xing, M. P. Verzi, W. An, A. Vaquero, J. A. Tischfield, L. Serrano, SIRT7 mediates L1 elements transcriptional repression and their association with the nuclear lamina, Nucleic Acids Res. 47, 7870-7885 (2019).
27. E. Sebestyen, F. Marullo, F. Lucini, C. Petrini, A. Bianchi, S. Valsoni, I. Olivieri, L. Antonelli, F. Gregoretti, G. Oliva, F. Ferrari, C. Lanzuolo, SAMMY-seq reveals early alteration of heterochromatin and deregulation of bivalent genes in Hutchinson-Gilford Progeria Syndrome., Nat. Commun. 11, 6274 (2020).
28. N. Kubben, K. R. Brimacombe, M. Donegan, Z. Li, T. Misteli, A high-content imaging-based screening pipeline for the systematic identification of anti-progeroid compounds, Methods 96, 46-58 (2016).
29. O. Velazquez Camacho, C. Galan, K. Swist-Rosowska, R. Ching, M.

Gamalinda, F. Karabiber, I. De La Rosa-Velazquez, B. Engist, B. Koschorz, N. Shukeir, M. Onishi-Seebacher, S. van de Nobelen, T. Jenuwein, Major satellite repeat RNA stabilize heterochromatin retention of Suv39h enzymes by RNA-nucleosome association and RNA:DNA hybrid formation., Elife 6, e25293 (2017).
30. F. Marasca, S. Sinha, R. Vadala, B. Polimeni, V. Ranzani, E. M. Paraboschi, F. V. Burattin, M. Ghilotti, M. Crosti, M. L. Negri, S. Campagnoli, S. Notarbartolo, A. Sartore-Bianchi, S. Siena, D. Prati, G. Montini, G. Viale, O. Torre, S. Harari, R. Grifantini, G. Solda, S. Biffo, S. Abrignani, B. Bodega, LINE1 are spliced in non-canonical transcript variants to regulate T cell quiescence and exhaustion, Nat. Genet. 54, 180-193 (2022).
31. A. Kalota, L. Karabon, C. R. Swider, E. Viazovkina, M. Elzagheid, M. J. Damha, A. M. Gewirtz, 2′-deoxy-2′-fluoro-beta-D-arabinonucleic acid (2′F-ANA) modified oligonucleotides (ON) effect highly efficient, and persistent, gene silencing., Nucleic Acids Res. 34, 451-61 (2006).
32. M. Takahashi, H. Li, J. Zhou, P. Chomchan, V. Aishwarya, M. J. Damha, J. J. Rossi, Dual Mechanisms of Action of Self-Delivering, Anti-HIV-1 FANA Oligonucleotides as a Potential New Approach to HIV Therapy, Mol. Ther.—Nucleic Acids 17, 615-625 (2019).
33. S. S. Teves, S. Henikoff, Salt fractionation of nucleosomes for genome-wide profiling., Methods Mol. Biol. 833, 421-32 (2012).
34. C. Herrmann, D. Avgousti, M. Weitzman, Differential Salt Fractionation of Nuclei to Analyze Chromatin-associated Proteins from Cultured Mammalian Cells, BIO-PROTOCOL 7 (2017), doi:10.21769/bioprotoc.2175.
35. D. K. Shumaker, T. Dechat, A. Kohlmaier, S. A. Adam, M. R. Bozovsky, M. R. Erdos, M. Eriksson, A. E. Goldman, S. Khuon, F. S. Collins, T. Jenuwein, R. D. Goldman, Mutant nuclear lamin A leads to progressive alterations of epigenetic control in premature aging, Proc. Natl. Acad. Sci. 103, 8703-8708 (2006).
36. W. Arancio, G. Pizzolanti, S. I. Genovese, M. Pitrone, C. Giordano, Epigenetic Involvement in Hutchinson-Gilford Progeria Syndrome: A Mini-Review, Gerontology 60, 197-203 (2014).
37. M. E. Levine, A. T. Lu, A. Quach, B. H. Chen, T. L. Assimes, S. Bandinelli, L. Hou, A. A. Baccarelli, J. D. Stewart, Y. Li, E. A. Whitsel, J. G. Wilson, A. P. Reiner1, A. Avivl, K. Lohman, Y. Liu, L. Ferrucci, S. Horvath, An epigenetic biomarker of aging for lifespan and healthspan, Aging (Albany. NY). (2018), doi:10.18632/aging.101414.
38. S. Horvath, J. Oshima, G. M. Martin, A. T. Lu, A. Quach, H. Cohen, S. Felton, M. Matsuyama, D. Lowe, S. Kabacik, J. G. Wilson, A. P. Reiner, A. Maierhofer, J. Flunkert, A. Aviv, L. Hou, A. A. Baccarelli, Y. Li, J. D. Stewart, E. A. Whitsel, L. Ferrucci, S. Matsuyama, K. Raj, Epigenetic clock for skin and blood cells applied to Hutchinson Gilford Progeria Syndrome and ex vivo studies, Aging (Albany. NY). (2018), doi:10.18632/aging.101508.
39. S. Horvath, Erratum to: DNA methylation age of human tissues and cell types, Genome Biol. 16, 96 (2015).
40. A. Maierhofer, J. Flunkert, J. Oshima, G. M. Martin, T. Haaf, S. Horvath, Accelerated epigenetic aging in Werner syndrome, Aging (Albany. NY). 9, 1143-1152 (2017).
41. S. G. Young, S. H. Yang, B. S. J. Davies, H.-J. Jung, L. G. Fong, Targeting Protein Prenylation in Progeria, Sci. Transl. Med. 5, 171ps3-171ps3 (2013).
42. M. Puttaraju, M. Jackson, S. Klein, A. Shilo, C. F. Bennett, L. Gordon, F. Rigo, T. Misteli, Systematic screening identifies therapeutic antisense oligonucleotides for Hutchinson-Gilford progeria syndrome, Nat. Med. 27, 526-535 (2021).
43. M. F. Cesta, Normal Structure, Function, and Histology of the Spleen, Toxicol. Pathol. 34, 455-465 (2006).
44. R. S. KURBAN, J. BHAWAN, Histologic Changes in Skin Associated with Aging, J. Dermatol. Surg. Oncol. 16, 908-914 (1990).
45. M. A. Merideth, L. B. Gordon, S. Clauss, V. Sachdev, A. C. M. Smith, M. B. Perry, C. C. Brewer, C. Zalewski, H. J. Kim, B. Solomon, B. P. Brooks, L. H. Gerber, M. L. Turner, D. L. Domingo, T. C. Hart, J. Graf, J. C. Reynolds, A. Gropman, J. A. Yanovski, M. Gerhard-Herman, F. S. Collins, E. G. Nabel, R. O. Cannon, W. A. Gahl, W. J. Introne, Phenotype and Course of Hutchinson-Gilford Progeria Syndrome, N. Engl. J. Med. 358, 592-604 (2008).
46. J. G. Fleischer, R. Schulte, H. H. Tsai, S. Tyagi, A. Ibarra, M. N. Shokhirev, L. Huang, M. W. Hetzer, S. Navlakha, Predicting age from the transcriptome of human dermal fibroblasts, Genome Biol. 19, 221 (2018).
47. A. Saleh, A. Macia, A. R. Muotri, Transposable Elements, Inflammation, and Neurological Disease, Front. Neurol. 10 (2019), doi:10.3389/fneur.2019.00894.
48. J. M. Sedivy, J. A. Kreiling, N. Neretti, M. De Cecco, S. W. Criscione, J. W. Hofmann, X. Zhao, T. Ito, A. L. Peterson, Death by transposition—the enemy within?, BioEssays 35, 1035-1043 (2013).
49. C. D. Sohl, M. R. Szymanski, A. C. Mislak, C. K. Shumate, S. Amiralaei, R. F. Schinazi, K. S. Anderson, Y. W. Yin, Probing the structural and molecular basis of nucleotide selectivity by human mitochondrial DNA polymerase 7, Proc. Natl. Acad. Sci. 112, 8596-8601 (2015).
50. L. Tang, S. Yang, H. Wang, H. Gu, X. Xia, Y. Feng, Z. Yang, S. Zhao, C. Su, Z. Su, K. Wang, Nucleoside reverse transcriptase inhibitor-induced rat oocyte dysfunction and low fertility mediated by autophagy, Oncotarget 9, 3895-3907 (2018).
51. W. Lewis, B. J. Day, W. C. Copeland, Mitochondrial toxicity of nrti antiviral drugs: an integrated cellular perspective, Nat. Rev. Drug Discov. 2, 812-822 (2003).
52. S. H. Yang, S. Y. Chang, Y. Tu, G. W. Lawson, M. O. Bergo, L. G. Fong, S. G. Young, Severe hepatocellular disease in mice lacking one or both CaaX prenyltransferases, J. Lipid Res. 53, 77-86 (2012).
53. B. S. J. Davies, R. H. Barnes, Y. Tu, S. Ren, D. A. Andres, H. P. Spielmann, J. Lammerding, Y. Wang, S. G. Young, L. G. Fong, An accumulation of non-farnesylated prelamin A causes cardiomyopathy but not progeria, Hum. Mol. Genet. 19, 2682-2694 (2010).
54. J. Couzin-Frankel, Drug Trial Offers Uncertain Start in Race to Save Children With Progeria, Science (80-.). 337, 1594-1595 (2012).
55. M. R. Erdos, W. A. Cabral, U. L. Tavarez, K. Cao, J. Gvozdenovic-Jeremic, N. Narisu, P. M. Zerfas, S. Crumley, Y. Boku, G. Hanson, D. V. Mourich, R. Kole, M. A. Eckhaus, L. B. Gordon, F. S. Collins, A targeted antisense therapeutic approach for Hutchinson-Gilford progeria syndrome, Nat. Med. 27, 536-545 (2021).
56. E. Beyret, H.-K. Liao, M. Yamamoto, R. Hernandez-Benitez, Y. Fu, G.

Erikson, P. Reddy, J. C. Izpisua Belmonte, Single-dose CRISPR-Cas9 therapy extends lifespan of mice with Hutchinson-Gilford progeria syndrome, Nat. Med. 25, 419-422 (2019).
57. O. Santiago-Fernindez, F. G. Osorio, V. Quesada, F. Rodriguez, S. Basso, D. Maeso, L. Rolas, A. Barkaway, S. Nourshargh, A. R. Folgueras, J. M. P. Freije, C. Löpez-Otin, Development of a CRISPR/Cas9-based therapy for Hutchinson-Gilford progeria syndrome, Nat. Med. 25, 423-426 (2019).
58. K. Suzuki, M. Yamamoto, R. Hernandez-Benitez, Z. Li, C. Wei, R. D. Soligalla, E. Aizawa, F. Hatanaka, M. Kurita, P. Reddy, A. Ocampo, T. Hishida, M. Sakurai, A. N. Nemeth, E. Nunez Delicado, J. M. Campistol, P. Magistretti, P. Guillen, C. Rodriguez Esteban, J. Gong, Y. Yuan, Y. Gu, G.-H. Liu, C. Löpez-Otin, J. Wu, K. Zhang, J. C. Izpisua Belmonte, Precise in vivo genome editing via single homology arm donor mediated intron-targeting gene integration for genetic disease correction, Cell Res. 29, 804-819 (2019).
59. L. W. Koblan, M. R. Erdos, C. Wilson, W. A. Cabral, J. M. Levy, Z.-M. Xiong, U. L. Tavarez, L. M. Davison, Y. G. Gete, X. Mao, G. A. Newby, S. P. Doherty, N. Narisu, Q. Sheng, C. Krilow, C. Y. Lin, L. B. Gordon, K. Cao, F. S. Collins, J. D. Brown, D. R. Liu, In vivo base editing rescues Hutchinson-Gilford progeria syndrome in mice, Nature 589, 608-614 (2021).
60. C. Bi, L. Wang, B. Yuan, X. Zhou, Y. Li, S. Wang, Y. Pang, X. Gao, Y. Huang, M. Li, Long-read individual-molecule sequencing reveals CRISPR-induced genetic heterogeneity in human ESCs., Genome Biol. 21, 213 (2020).
61. F. Della Valle, M. P. Thimma, M. Caiazzo, S. Pulcrano, M. Celii, S. A. Adroub, P. Liu, G. Alanis-Lobato, V. Broccoli, V. Orlando, Transdifferentiation of Mouse Embryonic Fibroblasts into Dopaminergic Neurons Reactivates LINE-1 Repetitive Elements, Stem Cell Reports 14, 60-74 (2020).
62. T. P. Naas, An actively retrotransposing, novel subfamily of mouse L1 elements, EMBO J. 17, 590-597 (1998).
63. N. H. El Said, F. Della Valle, P. Liu, A. Paytuvi-Gallart, S. Adroub, J. Gimenez, V. Orlando, Malat-1-PRC2-EZH1 interaction supports adaptive oxidative stress dependent epigenome remodeling in skeletal myotubes, Cell Death Dis. 12, 850 (2021).
64. P. Liu, M. Shuaib, H. Zhang, S. Nadeef, V. Orlando, Ubiquitin ligases HUWE1 and NEDD4 cooperatively control signal-dependent PRC2-Ezh1α/β-mediated adaptive stress response pathway in skeletal muscle cells, Epigenetics Chromatin 12, 78 (2019).
65. B. Fallatah, M. Shuaib, S. Adroub, A. Paytuvi-Gallart, F. Della Valle, S. Nadeef, C. Lanzuolo, V. Orlando, Ago1 controls myogenic differentiation by regulating eRNA-mediated CBP-guided epigenome reprogramming, Cell Rep. 37, 110066 (2021).
66. T. J. Triche, D. J. Weisenberger, D. Van Den Berg, P. W. Laird, K. D. Siegmund, Low-level processing of Illumina Infinium DNA Methylation BeadArrays, Nucleic Acids Res. 41, e90-e90 (2013).
67. J. R. Wisniewski, A. Zougman, N. Nagaraj, M. Mann, Universal sample preparation method for proteome analysis, Nat. Methods (2009), doi:10.1038/nmeth.1322.
68. F. Meier, A. D. Brunner, M. Frank, A. Ha, I. Bludau, E. Voytik, S. Kaspar-Schoenefeld, M. Lubeck, O. Raether, N. Bache, R. Aebersold, B. C. Collins, H. L. Rost, M. Mann, diaPASEF: parallel accumulation-serial fragmentation combined with data-independent acquisition, Nat. Methods (2020), doi:10.1038/s41592-020-00998-0.
69. W. R. Yang, D. Ardeljan, C. N. Pacyna, L. M. Payer, K. H. Burns, SQuIRE reveals locus-specific regulation of interspersed repeat expression, Nucleic Acids Res. 47, e27-e27 (2019).
70. F. Debacq-Chainiaux, J. D. Erusalimsky, J. Campisi, O. Toussaint, Protocols to detect senescence-associated beta-galactosidase (SA-βgal) activity, a biomarker of senescent cells in culture and in vivo, Nat. Protoc. 4, 1798-1806 (2009).

What is claimed is:

1. An oligonucleotide consisting of the sequence of SEQ ID NO: 1, wherein at least one nucleoside is 2′ fluoroarabinonucleic acid (FANA) modified.

2. The oligonucleotide of claim 1, wherein at least two nucleotides are 2′ FANA modified.

3. The oligonucleotide of claim 1 or 2, wherein 3 to 21 nucleosides are 2′ FANA modified.

4. The oligonucleotide of any one of claims 1-3, wherein at least ten nucleosides are 2′ FANA modified.

5. The oligonucleotide of any one of claims 1-4, wherein 21 nucleosides are 2′ FANA modified.

6. The oligonucleotide of any one of claims 1-5, wherein the at least two 2′ FANA modified nucleosides are located at a 5′ end or a 3′ end or both of the oligonucleotide.

7. The oligonucleotide of claim 6, wherein five 2′ FANA modified nucleosides are located at the 5′ end of the oligonucleotide and five 2′ FANA modified nucleosides are located at the 3′ end of the oligonucleotide and the remaining nucleotides are not 2′ FANA modified.

8. The oligonucleotide of any one of claims 1-7, wherein the oligonucleotide comprises at least one alternative internucleoside linkage.

9. The oligonucleotide of claim 8, wherein the at least one alternative internucleoside linkage is selected from a phosphorothioate internucleoside linkage and an alkyl phosphate internucleoside linkage.

10. The oligonucleotide of any one of claims 1-9, wherein the oligonucleotide comprises at least one alternative nucleobase.

11. The oligonucleotide of claim 10, wherein the at least one alternative nucleobase is selected from a 5-substituted pyrimidine, 6-azapyrimidine, pseudouridine, N-2 substituted purine, N-6 substituted purine, and 0-6 substituted purine.

12. The oligonucleotide of claim 11, wherein the at least one alternative nucleobase is 5′-methylcytosine or 5′methoxyuridine.

13. A pharmaceutical composition comprising one or more oligonucleotides of any one of claims 1-12 and a pharmaceutically acceptable carrier or excipient.

14. A composition comprising one or more of the oligonucleotides of any one of claims 1-12 and a lipid nanoparticle, a polyplex nanoparticle, a lipoplex nanoparticle, or a liposome.

15. A polynucleotide comprising a nucleotide sequence consisting of one or more of the oligonucleotides of any one of claims 1-12 and further comprising a regulatory nucleotide sequence that controls expression of the one or more oligonucleotide.

16. A vector comprising the polynucleotide of claim 15.

17. The vector of claim 16, wherein the vector is selected from a DNA plasmid, a viral vector, a bacterial vector, a cosmid, or an artificial chromosome.

18. A method of treating, preventing, or inhibiting premature aging or an age-related disease in a subject, the method comprising administering a therapeutically effective amount of an oligonucleotide of any of claims 1-12, a pharmaceutical composition of claim 13, or a composition of claim 14 to the subject in an amount effective to treat, prevent, or inhibit premature aging or an age-related disease in the subject.

19. The method of claim 18, wherein the subject has a progeroid syndrome.

20. The method of claim 19, the progeroid syndrome is selected from the group consisting of Hutchinson-Gilford progeria syndrome; Werner syndrome; atypical progeria, mandibuloacral dysplasia type A; mandibuloacral dysplasia type B; mandibuloacral dysplasia associated to MTX2; MDPL (mandibular hypoplasia, deafness, progeroid features and lipodystrophy syndrome); Nestor-Guillermo progeria syndrome; restrictive dermopathy, and other disorders with nuclear envelope abnormalities.

21. The method of claim 20, wherein the subject has a mutation in a LMNA gene, ZMPSTE24 gene, BANF1 gene, POLD1 gene, MTX2 gene, or WRN gene.

22. The method of claim 18, further comprising administering to the subject an additional therapeutic agent.

23. The method of claim 22, wherein the additional therapeutic agent is an aging-related therapeutic agent.

24. The method of any one of claims 18-23, wherein the therapeutically effective amount is administered by an intravenous, intramuscular, intradermal, subcutaneous, intraperitoneal, or intranasal route.

25. A method of treating, preventing, or inhibiting signs or symptoms of aging in a subject, the method comprising administering a therapeutically effective amount of an oligonucleotide of any of claims 1-12, a pharmaceutical composition of claim 13, or a composition of claim 14 to the subject in an amount effective to treat, prevent, or inhibit signs or symptoms of aging in the subject.

26. The method of claim 25, further comprising administering an additional therapeutic agent.

27. The method of claim 26, wherein the additional therapeutic agent is an aging-related therapeutic agent.

28. The method of any one of claims 25-27, wherein the therapeutically effective amount is administered by an intravenous, intramuscular, intradermal, subcutaneous, intraperitoneal, or intranasal route.

29. A method of decreasing LINE-1 RNA in a subject, the method comprising administering a therapeutically effective amount of an oligonucleotide of any of claims 1-12, a pharmaceutical composition of claim 13, or a composition of claim 14 to the subject.

30. The method of claim 29, further comprising administering an additional therapeutic agent.

31. The method of claim 30, wherein the additional therapeutic agent is an aging-related therapeutic agent.

32. The method of any one of claims 29-31, wherein the therapeutically effective amount is administered by an intravenous, intramuscular, intradermal, subcutaneous, intraperitoneal, or intranasal route.

33. A method of rejuvenating a tissue in a subject experiencing premature aging or having an age-related disease, the method comprising administering a therapeutically effective amount of an oligonucleotide of any of claims 1-12, a pharmaceutical composition of claim 13, or a composition of claim 14 to the subject.

34. The method of claim 33, further comprising administering an additional therapeutic agent.

35. The method of claim 34, wherein the additional therapeutic agent is an aging-related therapeutic agent.

36. The method of any one of claims 33-35, wherein the therapeutically effective amount is administered by an intravenous, intramuscular, intradermal, subcutaneous, intraperitoneal, or intranasal route.

37. A method of reversing signs or symptoms of aging in a tissue of a subject, the method comprising administering a therapeutically effective amount of an oligonucleotide of any of claims 1-12, a pharmaceutical composition of claim 13, or a composition of claim 14 to the subject.

38. The method of claim 37, further comprising administering an additional therapeutic agent.

39. The method of claim 38, wherein the additional therapeutic agent is an aging-related therapeutic agent.

40. The method of any one of claims 37-39, wherein the therapeutically effective amount is administered by an intravenous, intramuscular, intradermal, subcutaneous, intraperitoneal, or intranasal route.

41. The method of any one of claim 18-40, wherein the subject is a human.

42. The oligonucleotide of any one of claims 1-12, wherein the oligonucleotide exhibits at least 50% reduction in expression of senescence-associated secretory phenotype (SASP) genes in a progeroid human cell at a 1 μM single-stranded oligonucleotide concentration when determined using a human progeroid cell assay when compared with a control human progeroid cell.

43. The oligonucleotide of claim 42, wherein the progeroid human cell is a LMNA^{-/-} cells or a WRN^{-/-} cell.

44. A kit comprising:
(i) an oligonucleotide of any of claims 1-12, a pharmaceutical composition of claim 13, or a composition of claim 14 and
(ii) instructions for administering the oligonucleotide of any of claims 1-12, the pharmaceutical composition of claim 13, or the composition of claim 14 to a subject in need thereof.

45. The kit of claim 44, further comprising a device for administering the oligonucleotide of any of claims 1-12, the pharmaceutical composition of claim 13, or the composition of claim 14.


SEQUENCE LISTING (101 sequences)

SEQ ID NO: 1 - DNA, 21 bp, Homo sapiens
  source 1..21: mol_type = other DNA; note = human L1 2F-ANA ASO #1
  modified_base 1..5: mod_base = OTHER; note = 2' fluoroarabinonucleic acid
  modified_base 17..21: mod_base = OTHER; note = 2' fluoroarabinonucleic acid
acttcccttctcgcttcattt

SEQ ID NO: 2 - DNA, 21 bp, Mus musculus
  source 1..21: mol_type = other DNA; note = mouse L1 2F-ANA ASO #1
  modified_base 1..5: mod_base = OTHER; note = 2' fluoroarabinonucleic acid
  modified_base 19..21: mod_base = OTHER; note = 2' fluoroarabinonucleic acid
  modified_base 17: mod_base = OTHER; note = 2' fluoroarabinonucleic acid
ttgacctttctcccttactgc

SEQ ID NO: 3 - DNA, 21 bp, Mus musculus
  source 1..21: mol_type = other DNA; note = mouse L1 2F-ANA ASO #2
  modified_base 1..5: mod_base = OTHER; note = 2' fluoroarabinonucleic acid
  modified_base 16..20: mod_base = OTHER; note = 2' fluoroarabinonucleic acid
atatgttacttgacctttctc

SEQ ID NO: 4 - DNA, 21 bp, Mus musculus
  source 1..21: mol_type = other DNA; note = mouse L1 2F-ANA ASO #3
  modified_base 1..5: mod_base = OTHER; note = 2' fluoroarabinonucleic acid
  modified_base 17..21: mod_base = OTHER; note = 2' fluoroarabinonucleic acid
tgtaattctgataggccttcc

SEQ ID NO: 5 - DNA, 21 bp, Mus musculus
  source 1..21: mol_type = other DNA; note = mouse L1 2F-ANA ASO #4
  modified_base 1..4: mod_base = OTHER; note = 2' fluoroarabinonucleic acid
  modified_base 18..21: mod_base = OTHER; note = 2' fluoroarabinonucleic acid
agtgtctgtataacatctgtc

SEQ ID NO: 6 - DNA, 21 bp, Homo sapiens
  source 1..21: mol_type = other DNA; note = Human LINE-1 translation blocker LNA ASO #1
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
caaagatggggaaaaaacaga

SEQ ID NO: 7 - DNA, 21 bp, Homo sapiens
  source 1..21: mol_type = other DNA; note = human LINE-1 translation blocker LNA ASO#2
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
aacatcataatgacaggatca

SEQ ID NO: 8 - DNA, 23 bp, Mus musculus
  source 1..23: mol_type = other DNA; note = mouse Suv29H1
ctttgccacaagaaccatctggg

SEQ ID NO: 9 - DNA, 22 bp, Mus musculus
  source 1..22: mol_type = other DNA; note = mouse Suv29H1
gccaaagttggagtccattcgg

SEQ ID NO: 10 - DNA, 23 bp, synthetic construct
  source 1..23: mol_type = other DNA; note = mouse Suv39H1
ctttgccacaagaaccatctggg

SEQ ID NO: 11 - DNA, 22 bp, Mus musculus
  source 1..22: mol_type = other DNA; note = mouse SUV39H1
gccaaagttggagtccattcgg

SEQ ID NO: 12 - DNA, 20 bp, Mus musculus
  source 1..20: mol_type = other DNA; note = mouse p16
cgtgaacatgttgttgaggc

SEQ ID NO: 13 - DNA, 20 bp, Mus musculus
  source 1..20: mol_type = other DNA; note = mouse p16
gcagaagagctgctacgtga

SEQ ID NO: 14 - DNA, 20 bp, Mus musculus
  source 1..20: mol_type = other DNA; note = mouse p21
cggtgtcagagtctagggga

SEQ ID NO: 15 - DNA, 20 bp, Mus musculus
  source 1..20: mol_type = other DNA; note = mouse p21
atcaccaggattggacatgg

SEQ ID NO: 16 - DNA, 18 bp, Mus musculus
  source 1..18: mol_type = other DNA; note = mouse Atf3
ctctggccgttctctgga

SEQ ID NO: 17 - DNA, 20 bp, Mus musculus
  source 1..20: mol_type = other DNA; note = mouse Atf3
ggtcgcactgacttctgagg

SEQ ID NO: 18 - DNA, 19 bp, Mus musculus
  source 1..19: mol_type = other DNA; note = mouse Gadd45b
cggccaaactgatgaatgt

SEQ ID NO: 19 - DNA, 20 bp, Mus musculus
  source 1..20: mol_type = other DNA; note = mouse Gadd45b
tctgcagagcgatatcatcc

SEQ ID NO: 20 - DNA, 20 bp, Mus musculus
  source 1..20: mol_type = other DNA; note = mouse Btg2
gcgagcagagactcaaggtt

SEQ ID NO: 21 - DNA, 20 bp, Mus musculoides
  source 1..20: mol_type = other DNA; note = mouse Btg2
tagccagaacctttggatgg

SEQ ID NO: 22 - DNA, 20 bp, Mus musculus
  source 1..20: mol_type = other DNA; note = mouse Mmp13
tgatgaaacctggacaagca

SEQ ID NO: 23 - DNA, 20 bp, Mus musculus
  source 1..20: mol_type = other DNA; note = mouse Mmp13
ggtccttggagtgatccaga

SEQ ID NO: 24 - DNA, 23 bp, Mus musculus
  source 1..23: mol_type = other DNA; note = mouse Il1a
tctatgatgcaagctatggctca

SEQ ID NO: 25 - DNA, 19 bp, Mus musculus
  source 1..19: mol_type = other DNA; note = mouse Il1a
cggctctccttgaaggtga

SEQ ID NO: 26 - DNA, 22 bp, Mus musculus
  source 1..22: mol_type = other DNA; note = mouse LINE-1 A-family
tgagcactgaaactcagaggag

SEQ ID NO: 27 - DNA, 25 bp, Mus musculus
  source 1..25: mol_type = other DNA; note = mouse LINE-1 A-family
gattgttcttctggtgattctgtta

SEQ ID NO: 28 - DNA, 21 bp, Mus musculus
  source 1..21: mol_type = other DNA; note = mouse LINE-1 Gf-family
tgcccactgaaactaaggaga

SEQ ID NO: 29 - DNA, 23 bp, Mus musculus
  source 1..23: mol_type = other DNA; note = mouse LINE-1 Gf-family
gcttgttcttcaggtgactctgt

SEQ ID NO: 30 - DNA, 20 bp, Mus musculus
  source 1..20: mol_type = other DNA; note = mouse LINE-1 Tf-family
acagacgtaccttcctcacc

SEQ ID NO: 31 - DNA, 20 bp, Mus musculus
  source 1..20: mol_type = other DNA; note = mouse LINE-1 Tf-family
ccccatcacacccaagaaca

SEQ ID NO: 32 - DNA, 16 bp, Mus musculus
  source 1..16: mol_type = other DNA; note = mouse LINE-1 5'UTR
cagccggccaccttcc

SEQ ID NO: 33 - DNA, 18 bp, Mus musculus
  source 1..18: mol_type = other DNA; note = mouse LINE-1 5'UTR
ggtcccggaccaagatgg

SEQ ID NO: 34 - DNA, 18 bp, Mus musculus
  source 1..18: mol_type = other DNA; note = mouse Gapdh
cggccgcatcttcttgtg

SEQ ID NO: 35 - DNA, 18 bp, Mus musculus
  source 1..18: mol_type = other DNA; note = mouse Gapdh
gtgaccaggcgcccaata

SEQ ID NO: 36 - DNA, 21 bp, Mus musculus
  source 1..21: mol_type = other DNA; note = mouse LINE-1 3'UTR
agactgccatagccagggatc

SEQ ID NO: 37 - DNA, 22 bp, Mus musculus
  source 1..22: mol_type = other DNA; note = mouse LINE-1 3'UTR
cagctacatctgcgtcctttca

SEQ ID NO: 38 - DNA, 22 bp, Homo sapiens
  source 1..22: mol_type = other DNA; note = human Suv39H1
ccgcctactatggcaacatctc

SEQ ID NO: 39 - DNA, 23 bp, Homo sapiens
  source 1..23: mol_type = other DNA; note = human Suv39H1
cttgtggcaaagaaagcgatgcg

SEQ ID NO: 40 - DNA, 20 bp, Homo sapiens
  source 1..20: mol_type = other DNA; note = human p16
cccaacgcaccgaatagtta

SEQ ID NO: 41 - DNA, 18 bp, Homo sapiens
  source 1..18: mol_type = other DNA; note = human p16
accagcgtgtccaggaag

SEQ ID NO: 42 - DNA, 21 bp, Homo sapiens
  source 1..21: mol_type = other DNA; note = human p21
tggagactctcagggtcgaaa

SEQ ID NO: 43 - DNA, 22 bp, Homo sapiens
  source 1..22: mol_type = other DNA; note = human p21
ggcgtttggagtggtagaaatc

SEQ ID NO: 44 - DNA, 21 bp, Homo sapiens
  source 1..21: mol_type = other DNA; note = human Atf3
tgctcagagaagtcggaagaa

SEQ ID NO: 45 - DNA, 21 bp, Homo sapiens
  source 1..21: mol_type = other DNA; note = human Atf3
tggcacaaagttcatagggca

SEQ ID NO: 46 - DNA, 21 bp, Homo sapiens
  source 1..21: mol_type = other DNA; note = human Mmp13
actgagaggctccgagaaatg

SEQ ID NO: 47 - DNA, 19 bp, Homo sapiens
  source 1..19: mol_type = other DNA; note = human Mmp13
gaaccccgcatcttggctt

SEQ ID NO: 48 - DNA, 19 bp, Homo sapiens
  source 1..19: mol_type = other DNA; note = human Btg2
acgggaagggaaccgacat

SEQ ID NO: 49 - DNA, 19 bp, Homo sapiens
  source 1..19: mol_type = other DNA; note = human Btg2
acgggaagggaaccgacat

SEQ ID NO: 50 - DNA, 23 bp, Homo sapiens
  source 1..23: mol_type = other DNA; note = Human LINE-1 Ta-family
caaacaccgcatattctcactca

SEQ ID NO: 51 - DNA, 23 bp, Homo sapiens
  source 1..23: mol_type = other DNA; note = Human LINE-1 Ta-family
cttcctgtgtccatgtgatctca

SEQ ID NO: 52 - DNA, 20 bp, Homo sapiens
  source 1..20: mol_type = other DNA; note = human Gapdh
ttgaggtcaatgaaggggtc

SEQ ID NO: 53 - DNA, 20 bp, Homo sapiens
  source 1..20: mol_type = other DNA; note = human Gapdh
gaaggtgaaggtcggagtca

SEQ ID NO: 54 - DNA, 18 bp, Homo sapiens
  source 1..18: mol_type = other DNA; note = Human_chr1:2611417- 2907892
ccagaccccgtgtttccc

SEQ ID NO: 55 - DNA, 18 bp, Homo sapiens
  source 1..18: mol_type = other DNA; note = Human_chr1:2611417- 2907892
gacaaggacacccagggc

SEQ ID NO: 56 - DNA, 18 bp, Homo sapiens
  source 1..18: mol_type = other DNA; note = Human_chr2: 95932366- 96791133
agcccctctcacatccca

SEQ ID NO: 57 - DNA, 18 bp, Homo sapiens
  source 1..18: mol_type = other DNA; note = Human_chr2: 95932366- 96791133
gatggcttcagggtgggg

SEQ ID NO: 58 - DNA, 18 bp, Homo sapiens
  source 1..18: mol_type = other DNA; note = Human_chr2:240383252- 241590562
gcttggcaggtcccatgt

SEQ ID NO: 59 - DNA, 18 bp, Homo sapiens
  source 1..18: mol_type = other DNA; note = Human_chr2:240383252- 241590562
tgtggcagagtcagcagc

SEQ ID NO: 60 - DNA, 18 bp, Homo sapiens
  source 1..18: mol_type = other DNA; note = Human_chr4:610583- 2397676
cggctttgtgagtcccgt

SEQ ID NO: 61 - DNA, 18 bp, Homo sapiens
  source 1..18: mol_type = other DNA; note = Human_chr4:610583- 2397676
ggcttcactgaggcacgt

SEQ ID NO: 62 - DNA, 18 bp, Homo sapiens
  source 1..18: mol_type = other DNA
aagacacacctgctccgc

SEQ ID NO: 63 - DNA, 18 bp, Homo sapiens
  source 1..18: mol_type = other DNA; note = Human_chr6:168770242- 168797255
tggctcacagttccgcag

SEQ ID NO: 64 - DNA, 18 bp, Homo sapiens
  source 1..18: mol_type = other DNA; note = Human_chr6:170244713- 170485862
tgaaggtcacaggctgcg

SEQ ID NO: 65 - DNA, 18 bp, Homo sapiens
  source 1..18: mol_type = other DNA; note = Human_chr6:170244713- 170485862
ccacgctgaggttgcaga

SEQ ID NO: 66 - DNA, 18 bp, Homo sapiens
  source 1..18: mol_type = other DNA; note = Human_chr7:383506- 743563
cctggcgccttcctgaaa

SEQ ID NO: 67 - DNA, 18 bp, Homo sapiens
  source 1..18: mol_type = other DNA; note = Human_chr7:383506- 743563
gcgtgcagttccctcact

SEQ ID NO: 68 - DNA, 18 bp, Homo sapiens
  source 1..18: mol_type = other DNA; note = Human_chr7:27178748- 27233348
ctgttcggctgcccagaa

SEQ ID NO: 69 - DNA, 18 bp, Homo sapiens
  source 1..18: mol_type = other DNA; note = Human_chr7:27178748- 27233348
gtctggccggaaaaccga

SEQ ID NO: 70 - DNA, 18 bp, Homo sapiens
  source 1..18: mol_type = other DNA; note = Human_chr8:47000524- 47342037
caccccctcccacccata

SEQ ID NO: 71 - DNA, 18 bp, Homo sapiens
  source 1..18: mol_type = other DNA; note = Human_chr8:47000524- 47342037
tcaaaaccacctccgccc

SEQ ID NO: 72 - DNA, 18 bp, Homo sapiens
  source 1..18: mol_type = other DNA; note = Human_chr8:146053793- 146077272
ctgtctgccaccacccag

SEQ ID NO: 73 - DNA, 18 bp, Homo sapiens
  source 1..18: mol_type = other DNA; note = Human_chr8:146053793- 146077272
gaaggcaaccaccgagct

SEQ ID NO: 74 - DNA, 18 bp, Homo sapiens
  source 1..18: mol_type = other DNA; note = Human_chr10:102488677- 103002682
atccacaccaccaagccg

SEQ ID NO: 75 - DNA, 18 bp, Homo sapiens
  source 1..18: mol_type = other DNA; note = Human_chr10:102488677- 103002682
ggctgtggggattggctt

SEQ ID NO: 76 - DNA, 18 bp, Homo sapiens
  source 1..18: mol_type = other DNA; note = Human_chr11:2828602- 2855961
cagggcagtgacagggtg

SEQ ID NO: 77 - DNA, 18 bp, Homo sapiens
  source 1..18: mol_type = other DNA; note = Human_chr11:2828602- 2855961
atggtggtgcgtccttgg

SEQ ID NO: 78 - DNA, 18 bp, Homo sapiens
  source 1..18: mol_type = other DNA; note = Human_chr12: 132877550-133814303
gcagaagggacggggaac

SEQ ID NO: 79 - DNA, 18 bp, Homo sapiens
  source 1..18: mol_type = other DNA; note = Human_chr12: 132877550-133814303
gtccccacgtgcactacc

SEQ ID NO: 80 - DNA, 18 bp, Homo sapiens
  source 1..18: mol_type = other DNA; note = Human_chr14: 106053443-106388110
gcatgatggtgggtgcct

SEQ ID NO: 81 - DNA, 18 bp, Homo sapiens
  source 1..18: mol_type = other DNA; note = Human_chr14: 106053443-106388110
cagtcacacgggctggag

SEQ ID NO: 82 - DNA, 18 bp, Homo sapiens
  source 1..18: mol_type = other DNA; note = Human_chr15: 25292601- 25501678
acgcccactgaaggaagc

SEQ ID NO: 83 - DNA, 18 bp, Homo sapiens
  source 1..18: mol_type = other DNA; note = Human_chr15: 25292601- 25501678
ttgctcaggatcgctggc

SEQ ID NO: 84 - DNA, 18 bp, Homo sapiens
  source 1..18: mol_type = other DNA; note = Human_chr16: 884547- 910725
gtggacacaggggaaggc

SEQ ID NO: 85 - DNA, 18 bp, Homo sapiens
  source 1..18: mol_type = other DNA; note = Human_chr16: 884547- 910725
gtgtttctgggggacggg

SEQ ID NO: 86 - DNA, 18 bp, Homo sapiens
  source 1..18: mol_type = other DNA; note = Human_chr17: 36714636- 36736745
caggcagggtggaaggtg

SEQ ID NO: 87 - DNA, 18 bp, Homo sapiens
  source 1..18: mol_type = other DNA; note = Human_chr17: 36714636- 36736745
ggagggggatgaggacgt

SEQ ID NO: 88 - DNA, 18 bp, Homo sapiens
  source 1..18: mol_type = other DNA; note = Human_chr19: 19780869- 19869411
acagccacagccacttcc

SEQ ID NO: 89 - DNA, 18 bp, Homo sapiens
  source 1..18: mol_type = other DNA; note = Human_chr19: 19780869- 19869411
ctgtggccctgtgacctg

SEQ ID NO: 90 - DNA, 18 bp, Homo sapiens
  source 1..18: mol_type = other DNA; note = Human_chr22: 48981410- 49564632
gagggaggaaggggaggg

SEQ ID NO: 91 - DNA, 18 bp, Homo sapiens
  source 1..18: mol_type = other DNA; note = Human_chr22: 48981410- 49564632
acgtgaggtagggcgagt

SEQ ID NO: 92 - DNA, 18 bp, Homo sapiens
  source 1..18: mol_type = other DNA; note = Human_chrX: 58279640- 58319234
gcctgccttgacctctcc

SEQ ID NO: 93 - DNA, 18 bp, Homo sapiens
  source 1..18: mol_type = other DNA; note = Human_chrX: 58279640- 58319234
ggtgctctgccgtagtcc

SEQ ID NO: 94 - DNA, 9278 bp, synthetic construct
  source 1..9278: mol_type = other DNA; note = L1spa
ctttatagtattttgtacagttgccatattcgatatcctcctttttaatatcaaattgaggtaaagcaattttttccaactgaacaggatctcctgactgccagatgaatcttaaatcatcggttgtatatccaactacaataagtaaataaacaagttagaggaaaatatgcatatcaatgctttatccttatctcaataatccttattaaacacagaggaggtaagaaatcatgactttatgttatgaatttgtaacttaaagttcacagtggcctaatttttgccatgcacaaaatatcagcacaagaataacatgtccatccatccataaagaaacaataaattaaataatcatcaaaatcagatacaatcctagaccaccacgctagaaatatcttcttggccctatcataaaaaccgtgaaagtgagattgaagctcacggcacatttccatttataatcatggactatgtacctaaacttttaaaacaggtgtgattttaaataaatcactgccaggttcctggaagacatgcagaataagtggtcatcattatagcatcaatgaagtagcgaggaattcgtggcctggaattaggataggtggtatcaactcaaaactacacccagaaatgacctaacaaaagattgcaaagatcattggtttctaaaaacctacaaagtatttaaatgagtgtagtaagtgaatatagacttatttaaatctgtattaagcatattctaatatatatataatgtatatattatatatgtacatatatatggtactaaaacattgcatagacttaaacataatctcattgttagaaatcaacaaatatcattaaaagttaggattcactttagagcattaaaaaaccacaaaaccactaaataacatataatctaacaaacatacatagaatattttacaaggttgttataactcattaaactagcttaatctttaagcatctacactaaagattctttatgatatcagccttcaattatcaaggctgatggcttattattttctaaagataatcatcatagaaaattagtgaatatgttttatagaaatttcaggttataacatttagtacaacttttagaaactgaaaaacacaatcatagggacataaaatatacatctgtaaaaaataatttttctatccttccatagaaaatgcacactttgctgggcttacatatatttcaatgttttccattttttattatgagactattaacacagagcaattatattttagaagactataattttctatatattttccacactgccaatccaagttagtccagtcttcagaataagaagtacacagaccggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagatggcgaaaggtaaacggaggaatcttactaacaggaaccaagaccactcaccatcaccagaacccagcacacccacttcgcccagtccagggaaccccaacacacctgagaacctagacctagatttaaaagcatatctcatgatgatggtagagggcatcaagaaggactttaataaatcacttaaagaaatacaggagaacactgctaaagagttacaagtccttaaagaaaaacaggaaaacacaatcaaacaggtagaagtccttacagaaaaagaggaaaaaacatacaaacaggtgatggaaatgaacaaaaccatactagacctaaaaagggaagtagacacaataaagaaaactcaaagcgaggcaacactagagatagaaaccctaggaaagaaatctggaaccatagatttgagcatcagcaacagaatacaagagatggaagagagaatctcaggtgcagaacattccatagagaacatcggcacaacaatcaaagaaaatggaaaatgcaaaaagatcctaactcaaaatatccaggaaatccaggacacaataagaagaccaaacgtacggataataggagtggatgagaatgaagattttcaactcaaaggtccagcaaacatcttcaacaaaattattgaagaaaacttcccaaatctaaagaatgagatgcatatgaacatacaagaagcctacagaactccaaatagactggaccagaaaagaaattcctcccgacacataataatcagaacatcaaatgcactaaataaagatagaatactaaaagcagtaagggaaaaaggtcaagtaacatataaaggcaagcctatcagaattacaccagatttttcaccagagactatgaaagccagaagagcctggacagatgttatacagacactaagagaacacaaactgcagcccaggctactatacccagccaaactctcaattatcatagagggagaaaccaaagtattccacgacaaaaccaaattcacgcattatctctccacgaatccagcccttcaaaggataataacagaaaaaaaccaatacaagaacgggaacaacgccctagaaaaaacaagaaggtaatccctcaacaaacctaaaagaagacagccacaagaacagaatgccacctttaacaactaaaataacaggaagcaacaattacttttccttaatatctcttaacatcaatggtctcaactcgccaataaaaagacatagactaacaaactggctacacaaacaagacccaacattttgctgcttacaggaaactcatctcagagaaaaagatagacactacctcagaatgaaaggctggaaaacaattttccaagcaaatggtatgaagaaacaagcaggagtagccatcctaatatctgataagattgacttccaacccaaagtcatcaaaaaagacaaggagggacacttcattctcatcaaaggtaaaatcctccaagaggaactctcaattctgaatatctatgctccaaatacaagagcagccacattcactaaagaaactttagtaaagctcaaagcacacattgcgcctcacacaataatagtgggagacttcaacacaccactttcaccaatggacagatcatggaaacagaaactaaacagggacacactgaaactaacagaagtgatgaaacaaatggatctgacagatatctacagaacattttaccctaaaacaaaaggatataccttcttctcagcacctcatggtaccttctccaaaattgaccacataataggtcacaaatcaggcctcaacagattaaaaaatattgaaattgtcccatgtatcctatcagatcaccatgcactaaggctgatcttcaataacaaaataaataacagaaagccaacattcacatggaaactgaacaacactcttctcaatgataccttggtcaaggaaggaataaagaaagaaattaaagactttttagagtttaatgaaaatgaagccacaacgtacccaaacctttgggacacaatgaaagcatttctaagagggaaactcatagctatgagtgccttcaagaaaaaacgggagagagcacatactagcagcttgacaacacatctaaaagctctagaaaaaaaggaagcaaattcacccaagaggagtagacggcaggaaataatcaaactcaggggtgaaatcaaccaagtggaaacaagaagaactattcaaagaattaaccaaacgaggagttggttctttgagaaaatcaacaagatagataaacccttagctagactcactaaagggcacagggacaaaatcctaattaacaaaatcagaaatgaaaagggagacataacaacagatcctgaagaaatccaaaacaccatcagatccttctacaaaaggctatactcaacaaaactggaaaacctggacgaaatggacaaatttctggacagataccaggtaccaaagttgaatcaggatcaagttgaccttctaaacagtcccatatcccctaaagaaatagaagcagttattaatagtctcccagccaaaaaaagcccaggaccagacgggtttagtgcagagttctatcagaccttcaaagaagatctaactccagttctgcacaaactttttcacaagatagaagtagaaggtattctacccaactcattttatgaagccactattactctgatacctaaaccacagaaagatccaacaaagatagagaacttcagaccaatttctcttatgaacatcgatgcaaaaatccttaataaaattctcgctaaccgaatccaagaacacattaaagcaatcatccatcctgaccaagtaggttttattccagggatgcagggatggtttaatatacgaaaatccatcaatgtaatccattatataaacaaactcaaagacaaaaaccacatgatcatctcgttagatgcagaaaaagcatttgacaagatccaacacccattcatgataaaagttctggaaagatcaggaattcaaggccaatacctaaacatgataaaagcaatctacagcaaaccagtagccaacatcaaagtaaatggagagaagctggaagcaatcccactaaaatcagggactagacaaggctgcccactttctccctaccttttcaacatagtacttgaagtattagccagagcaattcgacaacaaaaggagatcaaggggatacaaattggaaaagaggaagtcaaaatatcactttttgcagatgatatgatagtatatataagtgaccctaaaaattccaacagagaactcctaaacctgataaacagcttcggtgaagtagctggatataaaattaactcaaacaagtcaatggcctttctctacacaaagaataaacaggctgagaaagaaattagggaaacaacacccttctcaatagccacaaataatataaaatatctcggcgtgactctaacgaaggaagtgaaagatctgtatgataaaaacttcaagtccctgaagaaagaaattaaagaagatctcagaagatggaaagatctcccatgctcatggattggcaggaccaacattgtaaaaatggctatcttgccaaaagcaatctacagattcaatgcaatccccattaaaattccaactcaattcttcaacgaattagaaggagcaatttgcaaattcatctggaataacaaaaaaccgaggatagcaaaaactcttctcaaggataaaagaacctctggtggaatcaccatgcctgacctaaagctttactacagagcaattgtgataaaaactgcatggtactggtatagagacagacaagtagaccaatggaatagaattgaagacccagaaatgaacccacacacctatggtcacttgatcttcgacaagggagccaaaaccatccagtggaagaaagacagcattttcaacaattggtgctggcacaactggttgttatcatgtagaagaatgcgaatcgatccatacttatctccttgtactaaggtcaaatctaagtggatcaaggaacttcacataaaaccagagacactgaaacttatagaggagaaagtggggaaaagtcttgaagatatgggcacaggggaaaaattcctgaacagaacagcaatggcttgtgctgtaagatcgagaattgacaaatgggacctaatgaaactccaaagtttctgcaaggcaaaagacactgtctataagacaaaaagaccaccaacagactgggaaaggatctttacctatcctaaatcagataggggactaatatccaacatatataaagaactcaagaaggtggacctcagaaaatcaaataacccccttaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtgagaatggctaagatcaaaaattcaggtgacagcagatgctggcgaggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagaagccccaactggtaagaaggacacatgctccactatgttcatagcagccttatttataatagccagaaactggaaagaacccagatgcccctcaacagaggaatggatacagaaaatgtggtacatctacacaatggagtactactcagctattaaaaagaatgaatttatgaaattcctagccaaatggatggacctggagagcatcatcctgagtgaggtaacacaatcacaaaggaactcacacaatatgtactcactgataagtggatactagcccaaaacctaggatacccacgatataagatacaatttcctaaacacatgaaactcaagaaaaatgaagactgaagtgtggacactatgcccctccttagaagtgggaacaaaacacccatggaaggagttacagaaacaaagtttggagctgagatgaaaggagggaccatgtagagactgccatatccagggatccaccccataatcagcatccaaacgctgacaccattgcatatactagcaagattttatcgaaaggacccagatgtagctgtctcttgtgagactatgccggggcctagcaaacacagaagtggatgctcacagtcagctaatggatggatcacagggctcccaatggaggagctagagaaagtacccaaggagctaaagggatctgcaaccctataggtggaacaacattatgaactaaccagtacccctgagctcttgactctagctgcatatgtatcaaaagatggcctagtcggccatcactggaaagagaggcccattggacacgcagactttgtgtgccccggtacaggggaacgccagggccaaaggggggggagtgggtgggtaggggagtgggggtgggtgggtaagggggacttttggtatagcattggaaatgtaaatgagctaaatacctaataaaaaatggaaaaaaaataaaaaaaataaaataaaaaaaaagaccatgaaacataaaaaaaaaaaaaaaaaaaaagaagtacacagactaacatcaactcgctaaaaagccaactacaatggaaagctctgtcttcttcaagattagcctatgcagtatgatgttagtatgttcaaataagcataatggtttttcacaataatagaatacattatctagatataaacattttattccccaaatcatttataacctcatttacatacagctctcaagttgcattttgcagcgttgtgtgtccatgggaaacagagttaagtccagaggacatgaaagtgtaatagacaacctgaaggcaagaacagaaaaattggaatcattgagcgtacatcgctgagatccactgacttgaaaaggaaactacaaataaacaagatctaagtagagcagggtacctcatgctcacaaggacgtc

SEQ ID NO: 95 - DNA, 9219 bp, synthetic construct
  source 1..9219: mol_type = other DNA; note = L1spa
ctttatagtattttgtacagttgccatattcgatatcctcctttttaatatcaaattgaggtaaagcaattttttccaactgaacaggatctcctgactgccagatgaatcttaaatcatcggttgtatatccaactacaataagtaaataaacaagttagaggaaaatatgcatatcaatgctttatccttatctcaataatccttattaaacacagaggaggtaagaaatcatgactttatgttatgaatttgtaacttaaagttcacagtggcctaatttttgccatgcacaaaatatcagcacaagaataacatgtccatccatccataaagaaacaataaattaaataatcatcaaaatcagatacaatcctagaccaccacgctagaaatatcttcttggccctatcataaaaaccgtgaaagtgagattgaagctcacggcacatttccatttataatcatggactatgtacctaaacttttaaaacaggtgtgattttaaataaatcactgccaggttcctggaagacatgcagaataagtggtcatcattatagcatcaatgaagtagcgaggaattcgtggcctggaattaggataggtggtatcaactcaaaactacacccagaaatgacctaacaaaagattgcaaagatcattggtttctaaaaacctacaaagtatttaaatgagtgtagtaagtgaatatagacttatttaaatctgtattaagcatattctaatatatatataatgtatatattatatatgtacatatatatggtactaaaacattgcatagacttaaacataatctcattgttagaaatcaacaaatatcattaaaagttaggattcactttagagcattaaaaaaccacaaaaccactaaataacatataatctaacaaacatacatagaatattttacaaggttgttataactcattaaactagcttaatctttaagcatctacactaaagattctttatgatatcagccttcaattatcaaggctgatggcttattattttctaaagataatcatcatagaaaattagtgaatatgttttatagaaatttcaggttataacatttagtacaacttttagaaactgaaaaacacaatcatagggacataaaatatacatctgtaaaaaataatttttctatccttccatagaaaatgcacactttgctgggcttacatatatttcaatgttttccattttttattatgagactattaacacagagcaattatattttagaagactataattttctatatattttccacactgccaatccaagttagtccagtcttcagaataagaagtacacagaccggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagatggcgaaaggtaaacggaggaatcttactaacaggaaccaagaccactcaccatcaccagaacccagcacacccacttcgcccagtccagggaaccccaacacacctgagaacctagacctagatttaaaagcatatctcatgatgatggtagagggcatcaagaaggactttaataaatcacttaaagaaatacaggagaacactgctaaagagttacaagtccttaaagaaaaacaggaaaacacaatcaaacaggtagaagtccttacagaaaaagaggaaaaaacatacaaacaggtgatggaaatgaacaaaaccatactagacctaaaaagggaagtagacacaataaagaaaactcaaagcgaggcaacactagagatagaaaccctaggaaagaaatctggaaccatagatttgagcatcagcaacagaatacaagagatggaagagagaatctcaggtgcagaacattccatagagaacatcggcacaacaatcaaagaaaatggaaaatgcaaaaagatcctaactcaaaatatccaggaaatccaggacacaataagaagaccaaacgtacggataataggagtggatgagaatgaagattttcaactcaaaggtccagcaaacatcttcaacaaaattattgaagaaaacttcccaaatctaaagaatgagatgcatatgaacatacaagaagcctacagaactccaaatagactggaccagaaaagaaattcctcccgacacataataatcagaacatcaaatgcactaaataaagatagaatactaaaagcagtaagggaaaaaggtcaagtaacatataaaggcaagcctatcagaattacaccagatttttcaccagagactgcccaggctactatacccagccaaactctcaattatcatagagggagaaaccaaagtattccacgacaaaaccaaattcacgcattatctctccacgaatccagcccttcaaaggataataacagaaaaaaaccaatacaagaacgggaacaacgccctagaaaaaacaagaaggtaatccctcaacaaacctaaaagaagacagccacaagaacagaatgccacctttaacaactaaaataacaggaagcaacaattacttttccttaatatctcttaacatcaatggtctcaactcgccaataaaaagacatagactaacaaactggctacacaaacaagacccaacattttgctgcttacaggaaactcatctcagagaaaaagatagacactacctcagaatgaaaggctggaaaacaattttccaagcaaatggtatgaagaaacaagcaggagtagccatcctaatatctgataagattgacttccaacccaaagtcatcaaaaaagacaaggagggacacttcattctcatcaaaggtaaaatcctccaagaggaactctcaattctgaatatctatgctccaaatacaagagcagccacattcactaaagaaactttagtaaagctcaaagcacacattgcgcctcacacaataatagtgggagacttcaacacaccactttcaccaatggacagatcatggaaacagaaactaaacagggacacactgaaactaacagaagtgatgaaacaaatggatctgacagatatctacagaacattttaccctaaaacaaaaggatataccttcttctcagcacctcatggtaccttctccaaaattgaccacataataggtcacaaatcaggcctcaacagattaaaaaatattgaaattgtcccatgtatcctatcagatcaccatgcactaaggctgatcttcaataacaaaataaataacagaaagccaacattcacatggaaactgaacaacactcttctcaatgataccttggtcaaggaaggaataaagaaagaaattaaagactttttagagtttaatgaaaatgaagccacaacgtacccaaacctttgggacacaatgaaagcatttctaagagggaaactcatagctatgagtgccttcaagaaaaaacgggagagagcacatactagcagcttgacaacacatctaaaagctctagaaaaaaaggaagcaaattcacccaagaggagtagacggcaggaaataatcaaactcaggggtgaaatcaaccaagtggaaacaagaagaactattcaaagaattaaccaaacgaggagttggttctttgagaaaatcaacaagatagataaacccttagctagactcactaaagggcacagggacaaaatcctaattaacaaaatcagaaatgaaaagggagacataacaacagatcctgaagaaatccaaaacaccatcagatccttctacaaaaggctatactcaacaaaactggaaaacctggacgaaatggacaaatttctggacagataccaggtaccaaagttgaatcaggatcaagttgaccttctaaacagtcccatatcccctaaagaaatagaagcagttattaatagtctcccagccaaaaaaagcccaggaccagacgggtttagtgcagagttctatcagaccttcaaagaagatctaactccagttctgcacaaactttttcacaagatagaagtagaaggtattctacccaactcattttatgaagccactattactctgatacctaaaccacagaaagatccaacaaagatagagaacttcagaccaatttctcttatgaacatcgatgcaaaaatccttaataaaattctcgctaaccgaatccaagaacacattaaagcaatcatccatcctgaccaagtaggttttattccagggatgcagggatggtttaatatacgaaaatccatcaatgtaatccattatataaacaaactcaaagacaaaaaccacatgatcatctcgttagatgcagaaaaagcatttgacaagatccaacacccattcatgataaaagttctggaaagatcaggaattcaaggccaatacctaaacatgataaaagcaatctacagcaaaccagtagccaacatcaaagtaaatggagagaagctggaagcaatcccactaaaatcagggactagacaaggctgcccactttctccctaccttttcaacatagtacttgaagtattagccagagcaattcgacaacaaaaggagatcaaggggatacaaattggaaaagaggaagtcaaaatatcactttttgcagatgatatgatagtatatataagtgaccctaaaaattccaacagagaactcctaaacctgataaacagcttcggtgaagtagctggatataaaattaactcaaacaagtcaatggcctttctctacacaaagaataaacaggctgagaaagaaattagggaaacaacacccttctcaatagccacaaataatataaaatatctcggcgtgactctaacgaaggaagtgaaagatctgtatgataaaaacttcaagtccctgaagaaagaaattaaagaagatctcagaagatggaaagatctcccatgctcatggattggcaggaccaacattgtaaaaatggctatcttgccaaaagcaatctacagattcaatgcaatccccattaaaattccaactcaattcttcaacgaattagaaggagcaatttgcaaattcatctggaataacaaaaaaccgaggatagcaaaaactcttctcaaggataaaagaacctctggtggaatcaccatgcctgacctaaagctttactacagagcaattgtgataaaaactgcatggtactggtatagagacagacaagtagaccaatggaatagaattgaagacccagaaatgaacccacacacctatggtcacttgatcttcgacaagggagccaaaaccatccagtggaagaaagacagcattttcaacaattggtgctggcacaactggttgttatcatgtagaagaatgcgaatcgatccatacttatctccttgtactaaggtcaaatctaagtggatcaaggaacttcacataaaaccagagacactgaaacttatagaggagaaagtggggaaaagtcttgaagatatgggcacaggggaaaaattcctgaacagaacagcaatggcttgtgctgtaagatcgagaattgacaaatgggacctaatgaaactccaaagtttctgcaaggcaaaagacactgtctataagacaaaaagaccaccaacagactgggaaaggatctttacctatcctaaatcagataggggactaatatccaacatatataaagaactcaagaaggtggacctcagaaaatcaaataacccccttaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtgagaatggctaagatcaaaaattcaggtgacagcagatgctggcgaggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagaagccccaactggtaagaaggacacatgctccactatgttcatagcagccttatttataatagccagaaactggaaagaacccagatgcccctcaacagaggaatggatacagaaaatgtggtacatctacacaatggagtactactcagctattaaaaagaatgaatttatgaaattcctagccaaatggatggacctggagagcatcatcctgagtgaggtaacacaatcacaaaggaactcacacaatatgtactcactgataagtggatactagcccaaaacctaggatacccacgatataagatacaatttcctaaacacatgaaactcaagaaaaatgaagactgaagtgtggacactatgcccctccttagaagtgggaacaaaacacccatggaaggagttacagaaacaaagtttggagctgagatgaaaggagggaccatgtagagactgccatatccagggatccaccccataatcagcatccaaacgctgacaccattgcatatactagcaagattttatcgaaaggacccagatgtagctgtctcttgtgagactatgccggggcctagcaaacacagaagtggatgctcacagtcagctaatggatggatcacagggctcccaatggaggagctagagaaagtacccaaggagctaaagggatctgcaaccctataggtggaacaacattatgaactaaccagtacccctgagctcttgactctagctgcatatgtatcaaaagatggcctagtcggccatcactggaaagagaggcccattggacacgcagactttgtgtgccccggtacaggggaacgccagggccaaaggggggggagtgggtgggtaggggagtgggggtgggtgggtaagggggacttttggtatagcattggaaatgtaaatgagctaaatacctaataaaaaatggaaaaaaaataaaaaaaataaaataaaaaaaaagaccatgaaacataaaaaaaaaaaaaaaaaaaaagaagtacacagactaacatcaactcgctaaaaagccaactacaatggaaagctctgtcttcttcaagattagcctatgcagtatgatgttagtatgttcaaataagcataatggtttttcacaataatagaatacattatctagatataaacattttattccccaaatcatttataacctcatttacatacagctctcaagttgcattttgcagcgttgtgtgtccatgggaaacagagttaagtccagaggacatgaaagtgtaatagacaacctgaaggcaagaacagaaaaattggaatcattgagcgtacatcgctgagatccactgacttgaaaaggaaactacaaataaacaagatctaagtagagcagggtacctcatgctcacaaggacgtc

SEQ ID NO: 96 - DNA, 20 bp, Mus musculus
  source 1..20: mol_type = other DNA; note = mouse LINE-1 A-family
gaatctgtctcccaggtctg

SEQ ID NO: 97 - DNA, 20 bp, Mus musculus
  source 1..20: mol_type = other DNA; note = mouse LINE-1 Gf-family
tgctaccctccaggtctgct

SEQ ID NO: 98 - DNA, 20 bp, Mus musculus
  source 1..20: mol_type = other DNA; note = mouse LINE-1 Tf-family
aaaaagagccctccttgtgc

SEQ ID NO: 99 - DNA, 14 bp, Mus musculus
  source 1..14: mol_type = other DNA; note = mouse LINE-1 5'UTR
cggaggacaggtgc

SEQ ID NO: 100 - DNA, 19 bp, Mus musculus
  source 1..19: mol_type = other DNA; note = mouse LINE-1 3'UTR
tgacaccattgcatacact

SEQ ID NO: 101 - DNA, 15 bp, Homo sapiens
  source 1..15: mol_type = other DNA; note = Human LINE-1 Ta-family
aggtgggaattgaac